(19) United States
(12) Patent Application Publication
(10) Pub. No.: US 2023/0009323 A1
(43) Pub. Date: Jan. 12, 2023

(54) COMPOUNDS AND METHODS FOR THE TREATMENT OF CRYPTOSPORIDIOSIS

(71) Applicants: The Broad Institute, Inc., Cambridge, MA (US); President and Fellows of Harvard College, Cambridge, MA (US); The University of Vermont and State Agriculture College, Burlington, VT (US)

(72) Inventors: Eamon COMER, Cambridge, MA (US); Bruno MELILLO, Cambridge, MA (US); Christopher D. HUSTON, Burlington, VT (US)

(73) Assignees: The Broad Institute, Inc., Cambridge, MA (US); President and Fellows of Harvard College, Cambridge, MA (US); The University of Vermont and State Agriculture College, Burlington, VT (US)

(21) Appl. No.: 17/770,264

(22) PCT Filed: Oct. 23, 2020
(86) PCT No.: PCT/US2020/057041

§ 371 Date: Apr. 19, 2022

Related U.S. Application Data
(60) Provisional application No. 62/926,090, filed on Oct. 25, 2019.

(51) Int. Cl. C07D 487/04; A61P 33/00

(52) U.S. Cl. CPC C07D 487/04; A61P 33/00

(57) ABSTRACT

Cryptosporidium is a leading contributor to early childhood mortality, and fully effective treatment for this important infectious disease is lacking. A bicyclic azetidine compound series is disclosed having potent in vitro activity against all C. parvum isolates tested, comparable potencies against C. hominis and C. parvum, and cured cryptosporidiosis in highly susceptible immunosuppressed mice with once-daily dosing.

### CROSS REFERENCE TO RELATED APPLICATIONS

The present application claims priority to U.S. App. No. 62/926,090, filed Oct. 25, 2019, which is hereby incorporated by reference in its entirety.

### STATEMENT OF RIGHTS TO INVENTIONS MADE UNDER FEDERALLY SPONSORED RESEARCH

This invention was made with government support under Grant Nos. R01AI112427 and R21AI101381 awarded by the National institute of Health. The government has certain rights in the invention.

### BACKGROUND

Cryptosporidiosis is a parasitic disease and is caused by Cryptosporidium, a genus of protozoan parasites in the phylum Apicomplexa. Cryptosporidiosis is most commonly caused by the intracellular apicomplexan parasites C. parvum and C. hominis. It may also be caused by C. canis, C. felis, C. meleagridis, and C. muris. Cryptosporidiosis affects the distal small intestine and can affect the respiratory tract in both immunocompetent and immunocompromised individuals. Cryptosporidiosis is one of the most common waterborne diseases and is found worldwide. It can also be transmitted to other animals, including cattle, sheep, pigs, horses, goats, and geckos. Nitazoxanide is the current standard of care for cryptosporidiosis, but the drug only exhibits partial efficacy in children and is no more effective than placebo in patients with AIDS.

The options for treatment of cryptosporidiosis are poor and no vaccines are available. A single drug, nitazoxanide, is currently approved for the treatment of cryptosporidiosis by the US FDA. Nitazoxanide modestly hastens recovery in immunocompetent individuals. However, it is not effective for the patients who need it most, malnourished children and those with compromised immune function. The recent appreciation of the full public health burden of cryptosporidiosis has invigorated the search for effective anti-Cryptosporidium treatment and stimulated the development of clear target product profiles, and the identification of multiple new Cryptosporidium inhibitors in stages of development ranging from early leads to pre-clinical candidates. Identification of additional lead compounds is critical, given the high attrition rate that is typical of drug development programs and the inevitability of future drug resistance.

### SUMMARY

In accordance with the foregoing objectives and others, the present disclosure provides compounds, compositions, and methods of treating diseases caused by parasites from the genus Cryptosporidium (e.g., cryptosporidiosis).

The compounds may have the structure of (IV):

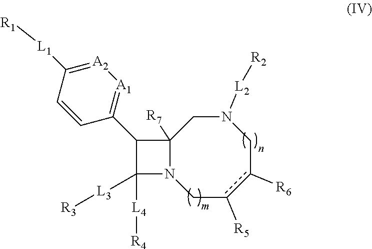

wherein the dashed bond ( [symbol] ) may be a single or double bond;
m is 0 (i.e., it is a bond) or 1;
n is 0, 1 or 2;
A₁ and A₂ are independently CH or N;
L₁ is absent (i.e., it is a bond), or —C≡C—;
L₂ is absent, alkylene (e.g., C₁-C₄ alkylene, methylene), heteroalkylene (e.g., C₁-C₄ heteroalkylene), —C(O)NR—; —SO₂—, or —C(O)—;
L₃ and L₄ are independently absent, alkylene (e.g., C₁-C₄ alkylene, methylene), or heteroalkylene (e.g., C₁-C₄ heteroalkylene);
R₁ is hydrogen, alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, C₁-C₈ heteroalkyl, C₁-C₅ heteroalkyl, C₃-C₁₂ heterocycloalkyl), halogen (e.g., fluoro, chloro), aryl (e.g., C₆-C₁₂ aryl, phenyl), or heteroaryl (e.g., C₅-C₁₂ heteroaryl, pyridinyl), and R₁ has one or more (e.g., two, three, four, five) optional points of substitution;
R₂ is perfluoroalkyl, aryl (e.g., C₆-C₁₂ aryl, phenyl), arylalkyl (e.g., C₇-C₁₄ alkylaryl, benzyl), alkyl (e.g., C₁-C₁₂ alkyl, C₁-C₈ alkyl, C₁-C₅ alkyl, C₃-C₁₂ cycloalkyl), or heteroaryl (e.g., C₅-C₁₂ heteroaryl, pyridinyl), and R₂ has one or more (e.g., two, three, four, five) optional points of substitution (e.g., with alkoxy, fluoroalkoxy);
R₃ and R₄ are independently hydrogen, —OH, —OR, —S(O)₂R, —N(R)S(O)₂R, —C(O)R, —N(R)C(O) R, —N(R)₂, or heterocyclyl, and R₃ and/or R₄ has one or more (e.g., two, three, four, five) optional points of substitution;
R₅ and R₆ are independently selected from hydrogen and —OH; wherein R₅ and R₆ are not each —OH;
R₇ is hydrogen, —CH₂OH, or —CH₂OR;
R is independently selected at each occurrence from hydrogen and alkyl (e.g., C₁-C₁₂ alkyl, C₁-C₈ alkyl, C₁-C₅ alkyl, C₃-C₁₂ cycloalkyl), wherein each R has one or more (e.g., two, three, four, five) optional points of substitution (e.g., with OH, with C(O)OH, —CN, —NH₂, —N(R^{A})₂); and
R^{A} is independently selected at each occurrence from hydrogen and lower alkyl (e.g., C₁-C₄ alkyl, methyl, ethyl, propyl, isopropyl); or
pharmaceutically acceptable salts thereof; or
prodrugs of any of the foregoing.

In some embodiments, -L₃-R₃ and/or -L₄-R₄ are not hydrogen. In various implementations, R₇ is-CH₂OR₈; wherein R₈ is alkyl (e.g., C₁-C₁₂ alkyl, C₁-C₈ alkyl, C₁-C₅ alkyl, C₃-C₁₂ cycloalkyl) having one or more (e.g., two, three, four, five) optional points of substitution (e.g., with OH, with C(O)OH, —CN, —NH₂, —N(R^{A})₂). For example, the compound may have the structure of formula (I):

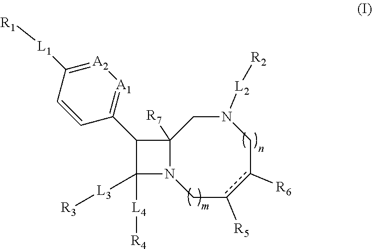

wherein the dashed bond ( [symbol] ) may be a single or double bond;
m is 0 (i.e., it is a bond) or 1;
n is 0, 1 or 2;
A₁ and A₂ are independently CH or N;
L₁ is absent (i.e., it is a bond), or —C≡C—;
L₂ is absent, alkylene (e.g., C₁-C₄ alkylene, methylene), —C(O)NR—; —SO₂—, or —C(O)—;
L₃ and L₄ are independently absent, alkylene (e.g., C₁-C₄ alkylene, methylene), or heteroalkylene (e.g., C₁-C₄ heteroalkylene);
R₁ is hydrogen, alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, C₁-C₈ heteroalkyl, C₁-C₅ heteroalkyl, C₃-C₁₂ heterocycloalkyl), halogen (e.g., fluoro, chloro), aryl (e.g., C₆-C₁₂ aryl, phenyl), heteroaryl (e.g., C₅-C₁₂ heteroaryl, pyridinyl), alkylaryl (e.g., C₇-C₁₄ alkylaryl, tolyl), arylalkyl (e.g., C₇-C₁₄ alkylaryl, benzyl), heteroalkylaryl (e.g., C₇-C₁₄ heteroalkylaryl), heteroarylalkyl (e.g., C₇-C₁₄ heteroarylalkyl), and R₁ has one or more (e.g., two, three, four, five) optional points of substitution;
R₂ is perfluoroalkyl, aryl (e.g., C₆-C₁₂ aryl, phenyl), arylalkyl (e.g., C₇-C₁₄ alkylaryl, benzyl), alkylaryl (e.g., C₇-C₁₄ alkylaryl, tolyl), alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, C₁-C₈ heteroalkyl, C₁-C₅ heteroalkyl, C₃-C₁₂ heterocycloalkyl), or heteroaryl (e.g., C₅-C₁₂ heteroaryl, pyridinyl), and R₂ has one or more (e.g., two, three, four, five) optional points of substitution (e.g., with alkoxy, fluoroalkoxy);
R₃ and R₄ are independently hydrogen, —OH, —OR, —S(O)₂R, —N(R)S(O)₂R, —C(O)R, —N(R)C(O) R, —N(R)₂, or heterocyclyl, and R₃ and/or R₄ has one or more (e.g., two, three, four, five) optional points of substitution;
R₅ and R₆ are independently selected from hydrogen and —OH;
R₇ is hydrogen, —CH₂OH, or —CH₂OR;
R is independently selected at each occurrence from hydrogen and alkyl (e.g., C₁-C₁₂ alkyl, C₁-C₈ alkyl, C₁-C₅ alkyl, C₃-C₁₂ cycloalkyl), wherein each R has one or more (e.g., two, three, four, five) optional points of substitution (e.g., with OH, with C(O)OH, —CN, —NH₂, —N(R_{A})₂); and
R_{A} is independently selected at each occurrence from hydrogen and lower alkyl (e.g., C₁-C₄ alkyl, methyl, ethyl, propyl, isopropyl);
wherein

a) -L₃-R₃ and -L₄-R₄ are each not hydrogen; and/or

b) R₇ is-CH₂OR₈; wherein R₈ is alkyl (e.g., C₁-C₁₂ alkyl, C₁-C₈ alkyl, C₁-C₅ alkyl, C₃-C₁₂ cycloalkyl) having one or more (e.g., two, three, four, five) optional points of substitution (e.g., with OH, with C(O)OH, —CN, —NH₂, —N(R_{A})₂); or

pharmaceutically acceptable salts thereof; or
prodrugs of any of the foregoing.

Pharmaceutical compositions are also provided, wherein the pharmaceutical composition may comprise one or more pharmaceutically acceptable carriers, diluents, or excipients and one or more compounds having the structure of formula (IV) (e.g., compounds having the structure of formula (I)).

Methods for the treatment or prophylaxis of these parasitic diseases in a subject in need thereof are also provided. The method may comprise administration of one or more compounds (typically in a therapeutically effective amount) as described herein to a subject in need thereof. In some embodiments, the subject is human. In other embodiments, the subject is not human (e.g., the pharmaceutical composition is formulated as a veterinary composition). In some embodiments, the subject is a mouse, rat, rabbit, non-human primate, lizards, geckos, cow, calf, sheep, lamb, horse, foal, pig, or piglet.

A method of inhibiting or preventing the growth of a population of parasites from the genus Cryptosporidium in a medium is also provided comprising contacting said population with a compound having the structure of formula (IV) (e.g., compounds having the structure of formula (I), (II), (III), (Ia), (IIa), (IIIa), and/or (IIIb)). In some embodiments, the medium is in vitro (e.g., cell culture medium such as DMEM or fibronectin). In some embodiments, the parasite population has infected a cultured cell. In some embodiments, the medium is in vivo (e.g., in a mouse model, in a human subject). In various embodiments, the Cryptosporidium parasites comprise wild type PheRS. In certain implementations, the Cryptosporidium parasites are C. parvum or C. hominis.

These and other aspects of the invention will be apparent to those skilled in the art from the following detailed description, which is simply, by way of illustration, various modes contemplated for carrying out the invention. As will be realized, the invention is capable of additional, different obvious aspects, all without departing from the invention. Accordingly, the specification is illustrative in nature and not restrictive.

### BRIEF DESCRIPTION OF FIGURES

FIG. 1 illustrates the structures of several compounds of the present disclosure which may be synthesized as described in Kato, N. et al. Nature 538, 344-349, doi:10.1038/nature19804 (2016) and Maetani, M. et al. J Am Chem Soc 139, 11300-11306, doi:10.1021/jacs.7b06994 (2017), each of which is hereby incorporated by reference in its entirety and particularly in relation to the synthetic schemes described therein.

FIGS. 2A-B show the correlation between in vitro activity against C. parvum and P. falciparum.

FIG. 2A demonstrates that the bicyclic azetidine series shows a strong correlation between blood-stage growth inhibition of Plasmodium falciparum: and C. parvum.

FIG. 2B has comparisons of SAR between P. falciparum and C. parvum growth inhibition showing similar trends, e.g. manipulation of the C4 position of the azetidine (Compound 2) results in a significant reduction in potency in both P. falciparum and C. parvum in vitro assays. Unmarked points (e.g., those represented by Compounds 3, 4, and 5) signify that broad chemical manipulation at this position is tolerated with respect to parasite potency. Circled points (e.g., those represented by Compounds 1 and 8) signify that limited chemical manipulation is tolerated at this position, and the Boxed point (represented by Compound 2) signifies that chemical manipulation at this position is not tolerated and leads to a significant loss of potency. The boxed EC₅₀ values are the EC₅₀ values for C. parvum.

FIG. 3 illustrates PD profiles for potent bicyclic azetidine compounds of the present disclosure. Bicyclic azetidines having the structure:

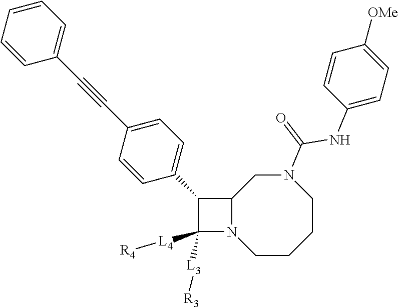

were formulated in 70% PEG400 and 30% aqueous glucose (5% in H₂O) or 10% ethanol, 4% Tween, 86% saline for intravenous and oral dosing and pharmacokinetics were determined in CD-1 mice as described in supplementary information C. parvum EC₅₀'s were obtained using C. parvum (Iowa) (10% FBS) BGF except Compound 16 which was obtained using C. parvum (Iowa) (1% FBS). Pf EC₅₀'s were obtained using the procedure disclosed in Bessoff, K. et al. Antimicrobial agents and chemotherapy 58, 2731-2739, doi:10.1128/AAC.02641-13 (2014), hereby incorporated by reference in its entirety. Reduction in C. parvum oocysts/mg feces was determined in the C. parvum immunocompromised mouse model as described in the Examples.

FIG. 4A is a schematic of NOD SCID gamma (NSG) mouse model of established intestinal cryptosporidiosis. Intestinal C. parvum infection was established by oral gavage of ˜10⁵ oocysts either 7 days (FIGS. 4B and C) or 14 days (FIG. 4D) prior to oral treatment with bicyclic azetidines, vehicle, or paromomycin (positive control). All data are the mean and SEM (n=4 mice per experimental group) of parasite shedding per mg of feces measured by qPCR with results for individual mice shown as superimposed data points. Data points for which no parasite DNA was detected are shown at the qPCR assay limit of detection (0.1 oocysts per mg feces).

FIG. 4B shows the in vivo efficacy of bicyclic azetidines with various pharmacokinetic characteristics. Mice received 50 mg·kg⁻¹·day⁻¹ of the indicated compound on days 7, 8, 9, and 10 of infection. Black bars=day 7 of infection; grey-colored bars=day 11 of infection.

FIG. 4C illustrates the results of the in vivo dose-response study with Compound 11. Compound 11 was administered at the indicated dose once daily on days 7, 8, 9, and 10 of infection. Abbreviations: mg per kg (mpk); day (D).

FIG. 4D shows the measured results of delayed dosing of Compound 11 with follow up to evaluate relapse. Compounds or vehicle were administered on days 14, 15, 16, and 17 of infection, and fecal oocyst shedding was monitored until day 28. Vehicle control (black squares and lines); Paromomycin 2,000 mg·kg⁻¹·day⁻¹ (light gray circles and lines); Compound 11 10 mg·kg⁻¹·day⁻¹ (grey triangles and lines). Each line indicates an individual mouse.

FIG. 5 illustrates the relationship between bioavailability and in vivo efficacy with Bicyclic azetidines via a plot of efficacy in the C. parvum immunocompromised mouse model (% reduction in oocysts/mg feces 24 h after final dose) vs bioavailability as determined in satellite PK study. The dashed line indicates the average change in oocyst shedding between days 7 and 11 of infection for the vehicle control mice (n=20).

FIGS. 6A-D illustrate how bicyclic azetidine Compound 11 blocks intracellular development and rapidly kills C. parvum.

FIG. 6A shows the effect of Compound 11 on C. parvum DNA synthesis. C. parvum infected HCT-8 cell monolayers were incubated with EdU in the presence or absence of EC₉₀ of Compound 11. Parasitophorous vacuoles (green), EdU-labeling (magenta). Arrows indicate selected parasites and EdU-labeled DNA. 40× dry objective (NA=0.7); scale=5 μm.

FIG. 6B illustrates the relative effects of Compound 11 on C. parvum asexual and sexual development. Dose-response studies were done either by labeling all parasite vacuoles with V. villosa lectin following Compound 11 treatment from 0-48 h of culture (asexual development, black circles and line), or by staining for the sexual stage marker DMC-1 following treatment from 48-72 h of cultures (asexual to sexual stage conversion; magenta squares and line). Data are the mean and SEM for data averaged from 2 biological replicates. The EC₉₀ and 95% confidence intervals for each assay given.

FIG. 6C shows measured C. parvum time-kill curves. Compound 11 was added ˜24 h after infecting HCT-8 cell monolayers with Bunchgrass Farm oocysts, and parasites were stained and enumerated with high-content microscopy at the indicated time points. Different compound concentrations are indicated as follows: open squares (DMSO control, at ˜ 60% parasites per nucleus at 80 h); solid black circles (EC₅₀, at ˜ 60% parasites per nucleus at 80 h); open grey squares (EC₉₀, at ˜ 30% parasites per nucleus at 80 h); open triangles (3×EC₉₀); solid triangles (6×EC₉₀); solid circles and green line (9×EC₉₀); and open grey circles (12×EC₉₀). Data are the means and SD for 4 culture wells per time point and are representative of 4 independent experiments.

FIG. 6D illustrates the rate of parasite elimination. One-phase exponential decay curves fit to time-kill data after normalizing to the DMSO control at each time point (open squares (DMSO control at 100%); open grey squares and black line (EC₉₀); open triangles and cyan line (3×EC₉₀)). Data points are the means and SD for 4 culture wells per time point and are representative of 4 independent experiments.

FIGS. 7A-D illustrate the positioning of guide sequence and in vivo selection of mutant transgentic parasites using Compound 11.

FIG. 7A Partial multiple amino acid sequence alignment of PheRS from P. falciparum, C. parvum and C. hominis. Box indicates the conserved glycine residue.

FIG. 7B Nucleotide sequence of C. parvum pheRS showing the guide sequence (highlighted in grey), L482V mutation (boxed) and the shield mutation (AGG to AGA) at the PAM to prevent re-cutting of the repair DNA.

FIG. 7C illustrates the measurements of in vivo selection of mutant transgenic C. parvum using Compound 11.

FIG. 7D shows the sequencing results of mutant transgenic parasite before and after selection with Compound 11 illustrating complete removal of wt population.

FIGS. 8A-E illustrate measurements demonstrating that transgenic C. parvum expressing PheRS 482V mutation is resistant to Compound 11.

FIG. 8A is a diagram illustrating the genome-editing strategy used for generating transgenic C. parvum lines expressing the pheRS L482 or V482 at the endogenous locus. Map of the C. parvum PheRS locus, location of the gRNA cut, and the targeting DNA repair are shown. Primers and amplicon sizes of PCR to check integration are indicated. Sequences of the primers are listed in Table 2.

FIG. 8B is the nucleotide sequence of C. parvum pheRS showing the guide sequence (highlighted in grey), L482V mutation (boxed) and the shield mutation (AGG to AGA) at the PAM to prevent re-cutting of the repair DNA.

FIG. 8C is a representative PCR using fecal genomic DNA from mutant transgenic parasite. Both the wt (L482) and mut (V482) transgenic parasites showed the same amplicon sizes.

FIG. 8D is a sequencing electropherogram of PCR product sequenced from wt (left) and mutant (right) transgenic lines. The mutation site is indicated by a star.

FIG. 8E shows the in vitro inhibition of parasite growth within HCT-8 cells assessed by measuring luminescence in the presence of the indicated concentrations of Compound 11. Black line=parental WT (Bunchgrass); blue line=WT transgenic PheRS; pink line=Mutant transgenic PheRS. Data are mean and standard deviation (SD) of n=3 biological replica.

FIG. 9 illustrates that transgenic C. parvum expressing PheRS 482V mutation shows resistance to Compound 19. in vitro inhibition of parasite growth within HCT-8 cells was assessed by measuring luminescence in the presence of the indicated concentrations of Compound 19. WT transgenic PheRS and Mutant transgenic PheRS measurements are highlighted. Data are mean and standard deviation (SD) of n=3 biological replicates.

FIG. 10 shows the inhibitory response of Compound 11 on recombinant ChPheRS. Recombinant ChPheRS was purified and its aminoacylation activity was measured in the presence of increasing concentrations of Compound 11. Compound 11 inhibited ChPheRS activity with potency comparable to that measured for inhibition of parasite growth. Data are the mean and SD for three biological replicates.

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%. It will be understood that the sum of all weight % of individual components will not exceed 100%.

Throughout this description, various components may be identified having specific values or parameters, however, these items are provided as exemplary embodiments. Indeed, the exemplary embodiments do not limit the various aspects and concepts of the present disclosure as many comparable parameters, sizes, ranges, and/or values may be implemented. Unless otherwise specified, the terms “first,” “second,” and the like, “primary,” “secondary,” and the like, do not denote any order, quantity, or importance, but rather are used to distinguish one element from another.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

Typically, alkyl groups described herein refer to a branched or straight-chain monovalent saturated aliphatic hydrocarbon radical of 1-30 carbon atoms (e.g., 1-16 carbon atoms, 6-20 carbon atoms, 8-16 carbon atoms, or 4-18 carbon atoms, 4-12 carbon atoms). In some embodiments, the alkyl group may be substituted with 1, 2, 3, or 4 substituent groups as defined herein. Alkyl groups may have from 1-26 carbon atoms. In other embodiments, alkyl groups will have from 6-18 or from 1-8 or from 1-6 or from 1-4 or from 1-3 carbon atoms, including for example, embodiments having one, two, three, four, five, six, seven, eight, nine, or ten carbon atoms. Any alkyl group may be substituted or unsubstituted. Examples of alkyl groups include methyl, ethyl, propyl, butyl, pentyl, hexyl, heptyl, octyl, nonyl, decyl, undecyl, and dodecyl groups. In some embodiments, the alkyl group may be a cycloalkyl group such as, for example, a C₃-C₁₂ cycloalkyl including cyclopropyl, cyclobutyl, and cyclohexyl. Alkyl groups may be saturated or unsaturated (e.g., alkenyl, alkynyl, cycloalkenyl). Heteroalkyl groups may refer to branched or straight-chain monovalent saturated aliphatic hydrocarbon radicals with one or more heteroatoms (e.g., N, O, S) in the carbon chain. Heteroalkyl groups may have 1-30 carbon atoms (e.g., 1-16 carbon atoms, 6-20 carbon atoms, 8-16 carbon atoms, or 4-18 carbon atoms, 4-12 carbon atoms). In some embodiments, the heteroalkyl group may be substituted with 1, 2, 3, or 4 substituent groups as defined herein. Heteroalkyl groups may have from 1-26 carbon atoms. In other embodiments, heteroalkyl groups will have from 6-18 or from 1-8 or from 1-6 or from 1-4 or from 1-3 carbon atoms, including for example, embodiments having one, two, three, four, five, six, seven, eight, nine, or ten carbon atoms. In some embodiments, the heteroalkyl group can be further substituted with 1, 2, 3, or 4 substituent groups as described herein for alkyl groups. Examples of heteroalkyl groups are an alkoxy. Alkoxy substituent groups or alkoxy-containing substituent groups may be substituted by, for example, one or more alkyl groups. Alkyl groups may be saturated or unsaturated (e.g., alkenyl, alkynyl, cycloalkenyl). In some embodiments, the heteroalkyl group may be a heterocycloalkyl group such as, for example, a C₃-C₁₂ cycloalkyl including cyclopropyl, cyclobutyl, and cyclohexyl.

Aryl groups may be aromatic mono- or polycyclic radicals of 6 to 12 carbon atoms having at least one aromatic ring. Examples of such groups include, but are not limited to, phenyl, naphthyl, 1,2,3,4-tetrahydronaphthalyl, 1,2-dihydronaphthalyl, indanyl, and 1H-indenyl. Typically, heteroaryls include mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group. Examples of heteroaryl groups are pyridyl, benzooxazolyl, benzoimidazolyl, and benzothiazolyl.

Typically, hydrocarbon groups (e.g., alkyl, heteroalkyl, aryl, heteroaryl) ending in “ene” (e.g., alkylene, heteroalkylene, arylene, heteroarylene) are divalent groups having two points of connection to other portions of the compound. In various embodiments, these divalent groups may have from 1 to 12 carbon atoms or from 1 to 6 carbon atoms or from 1 to 4 carbon atoms. For example, an alkylene group may be a branched or unbranched C₁-C₁₂ or C₁-C₆ or C₁-C₄ alkylene including methylene (—CH₂—), ethylene (—CH₂CH₂—), and the propylene isomers (e.g., —CH₂CH₂CH₂— and —CH(CH₃)CH₂—). The divalent group may be substituted or unsubstituted.

The term “substituent” refers to a group “substituted” on, e.g., an alkyl, at any atom of that group, replacing one or more hydrogen atoms therein (e.g., the point of substitution). In some aspects, the substituent(s) on a group are independently any one single, or any combination of two or more of the permissible atoms or groups of atoms delineated for that substituent. In another aspect, a substituent may itself be substituted with any one of the substituents described herein. Substituents may be located pendant to the hydrocarbon chain.

A substituted hydrocarbon group may have as a substituent one or more hydrocarbon radicals, substituted hydrocarbon radicals, or may comprise one or more heteroatoms. Examples of substituted hydrocarbon radicals include, without limitation, heterocycles, such as heteroaryls. Unless otherwise specified, a hydrocarbon substituted with one or more heteroatoms will comprise from 1-20 heteroatoms. In other embodiments, a hydrocarbon substituted with one or more heteroatoms will comprise from 1-12 or from 1-8 or from 1-6 or from 1-4 or from 1-3 or from 1-2 heteroatoms. Examples of heteroatoms include, but are not limited to, oxygen, nitrogen, sulfur, phosphorous, halogen (e.g., F, Cl, Br, I), boron, silicon In some embodiments, heteroatoms will be selected from the group consisting of oxygen, nitrogen, sulfur, phosphorous, and halogen (e.g., F, Cl, Br, I). In some embodiments, a heteroatom or group may substitute a carbon. In some embodiments, a heteroatom or group may substitute a hydrogen. In some embodiments, a substituted hydrocarbon may comprise one or more heteroatoms in the backbone or chain of the molecule (e.g., interposed between two carbon atoms, as in “oxa”). In some embodiments, a substituted hydrocarbon may comprise one or more heteroatoms pendant from the backbone or chain of the molecule (e.g., covalently bound to a carbon atom in the chain or backbone, as in “oxo”).

In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Unless otherwise noted, all groups described herein (e.g., alkyl, cycloalkyl, heteroalkyl, heterocycloalkyl, aryl, heteroaryl, alkylene, heteroalkylene, cylcoalkylene, heterocycloalkylene) may optionally contain one or more common substituents, to the extent permitted by valency. Common substituents include halogen (e.g., F, Cl), C₁₋₁₂ straight chain or branched chain alkyl, C₂₋₁₂ alkenyl, C₂₋₁₂ alkynyl, C₃₋₁₂ cycloalkyl, C₆₋₁₂ aryl, C₃₋₁₂ heteroaryl, C₃₋₁₂ heterocyclyl, C₁₋₁₂ alkylsulfonyl, nitro, cyano, —COOR, —C(O)NRR′, —OR, —SR, —NRR′, and oxo, such as mono- or di- or tri-substitutions with moieties such as halogen, fluoroalkyl, perfluoroalkyl, perfluroalkoxy, trifluoromethoxy, chlorine, bromine, fluorine, methyl, methoxy, pyridyl, furyl, triazyl, piperazinyl, pyrazoyl, imidazoyl, and the like, each optionally containing one or more heteroatoms such as halo, N, O, S, and P. R and R′ are independently hydrogen, C₁₋₁₂ alkyl, C₁₋₁₂ haloalkyl, C₂₋₁₂ alkenyl, C₂₋₁₂ alkynyl, C₃₋₁₂ cycloalkyl, C₄₋₂₄ cycloalkylalkyl, C₆₋₁₂ aryl, C₇₋₂₄ aralkyl, C₃₋₁₂ heterocyclyl, C₃₋₂₄ heterocyclylalkyl, C₃₋₁₂ heteroaryl, or C₄₋₂₄ heteroarylalkyl. Further, as used herein, the phrase optionally substituted indicates the designated hydrocarbon group may be unsubstituted (e.g., substituted with H) or substituted. Typically, substituted hydrocarbons are hydrocarbons with a hydrogen atom removed and replaced by a substituent (e.g., a common substituent).

It is understood by one of ordinary skill in the chemistry art that substitution at a given atom is limited by valency. The use of a substituent (radical) prefix names such as alkyl without the modifier optionally substituted or substituted is understood to mean that the particular substituent is unsubstituted. However, the use of haloalkyl without the modifier optionally substituted or substituted is still understood to mean an alkyl group, in which at least one hydrogen atom is replaced by halo. Where a group may be substituted by one or more of a number of substituents, such substitutions are selected so as to comply with principles of chemical bonding with regard to valencies, and to give compounds which are not inherently unstable. For example, any carbon atom will be bonded to two, three, or four other atoms, consistent with the four valence electrons of carbon. Additionally, when a structure has less than the required number of functional groups indicated, those carbon atoms without an indicated functional group are bonded to the requisite number of hydrogen atoms to satisfy the valency of that carbon.

The term “pharmaceutical composition,” as used herein, represents a composition containing a compound described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap); for topical administration (e.g., as a cream, gel, lotion, or ointment); for intravenous administration (e.g., as a sterile solution free of particulate emboli and in a solvent system suitable for intravenous use); or in any other formulation described herein (see below).

As used herein, the phrase “pharmaceutically acceptable” generally safe for ingestion or contact with biologic tissues at the levels employed. Pharmaceutically acceptable is used interchangeably with physiologically compatible. It will be understood that the pharmaceutical compositions of the disclosure include nutraceutical compositions (e.g., dietary supplements) unless otherwise specified.

Unit dosage forms, also referred to as unitary dosage forms, often denote those forms of medication supplied in a manner that does not require further weighing or measuring to provide the dosage (e.g., tablet, capsule, caplet). For example, a unit dosage form may refer to a physically discrete unit suitable as a unitary dosage for human subjects and other mammals, each unit containing a predetermined quantity of active material calculated to produce the desired therapeutic effect, in association with any suitable pharmaceutical excipient or excipients. Exemplary, non-limiting unit dosage forms include a tablet (e.g., a chewable tablet), caplet, capsule (e.g., a hard capsule or a soft capsule), lozenge, film, strip, and gel cap. In certain embodiments, the compounds described herein, including crystallized forms, polymorphs, and solvates thereof, may be present in a unit dosage form.

Useful pharmaceutical carriers, excipients, and diluents for the preparation of the compositions hereof, can be solids, liquids, or gases. These include any and all solvents, dispersion media, coatings, antibacterial and antifungal agents, isotonic and absorption delaying agents and the like. The pharmaceutically acceptable carrier or excipient does not destroy the pharmacological activity of the disclosed compound and is nontoxic when administered in doses sufficient to deliver a therapeutic amount of the compound. Thus, the compositions can take the form of tablets, pills, capsules, suppositories, powders, enterically coated or other protected formulations (e.g., binding on ion-exchange resins or packaging in lipid-protein vesicles), sustained release formulations, solutions, suspensions, elixirs, and aerosols. The carrier can be selected from the various oils including those of petroleum, animal, vegetable or synthetic origin, e.g., peanut oil, soybean oil, mineral oil, and sesame oil. Water, saline, aqueous dextrose, and glycols are examples of liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, chitosan, talc, glucose, lactose, gelatin, malt, rice, flour, chalk, silica, magnesium stearate, sodium stearate, glycerol monostearate, sodium chloride, dried skim milk, glycerol, propylene glycol, water, and ethanol. The compositions may be subjected to conventional pharmaceutical additives such as preservatives, stabilizing agents, wetting or emulsifying agents, salts for adjusting osmotic pressure, and buffers. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for administration to the recipient.

Non-limiting examples of pharmaceutically acceptable carriers and excipients include sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide; alginic acid; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening, flavoring and perfuming agents; preservatives; antioxidants; ion exchangers; alumina; aluminum stearate; lecithin; self-emulsifying drug delivery systems (SEDDS) such as d-atocopherol polyethyleneglycol 1000 succinate; surfactants used in pharmaceutical dosage forms such as Tweens or other similar polymeric delivery matrices; serum proteins such as human serum albumin; glycine; sorbic acid; potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances; polyacrylates; waxes; and polyethylene-polyoxypropylene-block polymers. Cyclodextrins such as α-, β-, and γ-cyclodextrin, or chemically modified derivatives such as hydroxyalkylcyclodextrins, including 2- and 3-hydroxypropyl-cyclodextrins, or other solubilized derivatives can also be used to enhance delivery of the compounds described herein.

The compounds described herein may be present as a pharmaceutically acceptable salt. Typically, salts are composed of a related number of cations and anions (at least one of which is formed from the compounds described herein) coupled together (e.g., the pairs may be bonded ionically) such that the salt is electrically neutral. Pharmaceutically acceptable salts may retain or have similar activity to the parent compound (e.g., an ED₅₀ within 10%) and have a toxicity profile within a range that affords utility in pharmaceutical compositions. For example, pharmaceutically acceptable salts may be suitable for use in contact with the tissues of humans and animals without undue toxicity, irritation, allergic response and are commensurate with a reasonable benefit/risk ratio. Pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, dichloroacetate, digluconate, dodecylsulfate, ethanesulfonate, formate, fumarate, glucoheptonate, glutamate, glycerophosphate, hemisulfate, heptonate, hexanoate, hippurate, hydrobromide, hydrochloride, hydroiodide, 2-hydroxy-ethanesulfonate, isethionate, lactobionate, lactate, laurate, lauryl sulfate, malate, maleate, malonate, mandelate, methanesulfonate, mucate, 2-naphthalenesulfonate, nicotinate, nitrate, oleate, oxalate, palmitate, pamoate, pantothenate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative basic salts include alkali or alkaline earth metal salts include sodium, lithium, potassium, calcium, and magnesium, aluminum salts, as well as nontoxic ammonium, quaternary ammonium, and amine cations, including, but not limited to ammonium, tetramethylammonium, tetraethylammonium, methylamine, dimethylamine, trimethylamine, triethylamine, caffeine, and ethylamine.

Pharmaceutically acceptable acid addition salts of the disclosure can be formed by the reaction of a compound of the disclosure with an equimolar or excess amount of acid. Alternatively, hemi-salts can be formed by the reaction of a compound of the disclosure with the desired acid in a 2:1 ratio, compound to acid. The reactants are generally combined in a mutual solvent such as diethyl ether, tetrahydrofuran, methanol, ethanol, iso-propanol, benzene, or the like. The salts normally precipitate out of solution within, e.g., one hour to ten days and can be isolated by filtration or other conventional methods.

Compounds provided herein can have one or more asymmetric carbon atoms and can exist in the form of optically pure enantiomers, mixtures of enantiomers such as racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as chiral chromatography and separation methods based thereon. The appropriate technique and/or method for separating an enantiomer of a compound described herein from a racemic mixture can be readily determined by those of skill in the art. “Racemate” or “racemic mixture” means a mixture containing two enantiomers, wherein such mixtures exhibit no optical activity; i.e., they do not rotate the plane of polarized light. “Geometric isomer” means isomers that differ in the orientation of substituent atoms (e.g., to a carbon-carbon double bond, to a cycloalkyl ring, to a bridged bicyclic system). Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds disclosed herein may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99%, or 99.9%) by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either stereoisomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The disclosure embraces all of these forms.

Solvates of the compounds described herein may the aggregate of the compound or an ion of the compound with one or more solvents. Such solvents may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The crystalline form of the compounds described herein may refer to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

Amorphous forms of the compounds described herein may be solid forms substantially lacking three-dimensional order. In certain embodiments, an amorphous form of a solid is a solid form that is substantially not crystalline. In certain embodiments, the X-ray powder diffraction (XRPD) pattern of an amorphous form includes a wide scattering band with a peak at 2θ of, for example, from 20 to 70°, using CuKα radiation. In certain embodiments, the XRPD pattern of an amorphous form further includes one or more peaks attributed to crystalline structures. In certain embodiments, the maximum intensity of any one of the one or more peaks attributed to crystalline structures observed at a 20 of from 20 to 700 is not more than 300-fold, not more than 100-fold, not more than 30-fold, not more than 10-fold, or not more than 3-fold of the maximum intensity of the wide scattering band. In certain embodiments, the XRPD pattern of an amorphous form includes no peaks attributed to crystalline structures.

Polymorphs or polymorphic forms of the compounds may be crystalline forms of the compound (or a salt, hydrate, or solvate thereof). Typically, all polymorphic forms have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphic forms of a compound can be prepared by crystallization under different conditions.

The term “effective amount” or “therapeutically effective amount” of an agent (e.g compounds having the structure of formula (I)), as used herein, is that amount sufficient to effect beneficial or desired results, such as clinical results, and, as such, an “effective amount” depends upon the context in which it is being applied. In some embodiments, the compounds are administered in an effective amount for the treatment or prophylaxis of a disease disorder or condition. In another embodiment, in the context of administering an agent that is an anticryptosporoidal agent, an effective amount of an agent is, for example, an amount sufficient to achieve alleviation or amelioration or prevention or prophylaxis of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition (e.g., cryptosporidiosis); and remission (whether partial or total), whether detectable or undetectable, as compared to the response obtained without administration of the agent.

Typically, the treatment of a disease, disorder, or condition (e.g., the conditions described herein such as cryptosporidiosis) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

As used herein, the term “subject” refers to any organism to which a composition and/or compound in accordance with the disclosure may be administered, e.g., for experimental, diagnostic, prophylactic, and/or therapeutic purposes. Typical subjects include any animal (e.g., mammals such as mice, rats, rabbits, non-human primates, and humans). A subject in need thereof is typically a subject for whom it is desirable to treat a disease, disorder, or condition as described herein. For example, a subject in need thereof may seek or be in need of treatment, require treatment, be receiving treatment, may be receiving treatment in the future, or a human or animal that is under care by a trained professional for a particular disease, disorder, or condition.

Compounds

The present disclosure provides for compounds and pharmaceutical compositions useful for the treatment or prophylaxis of a parasitic disease caused by a parasite from the genus Cryptosporidium (e.g., C. hominis, C. parvum) such as cryptosporidiosis. The disclosure also provides methods of using these compounds and compositions, for example in the treatment or prophylaxis of a parasitic disease caused by a parasite from the genus Cryptosporidium (e.g., C. hominis, C. parvum) such as cryptosporidiosis or in the preparation of a medicament for the treatment or prophylaxis of a parasitic disease caused by a parasite from the genus Cryptosporidium (e.g., C. hominis, C. parvum) such as cryptosporidiosis.

The compounds may have the structure of (IV):

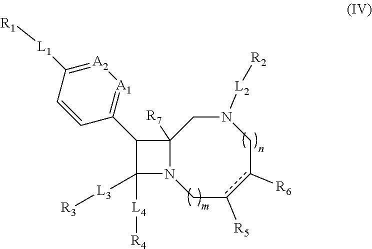

wherein the dashed bond ( [symbol] ) may be a single or double bond;
m is 0 (i.e., it is a bond) or 1;
n is 0, 1 or 2;
A₁ and A₂ are independently CH or N;
L₁ is absent (i.e., it is a bond), or —C≡C—;
L₂ is absent, alkylene (e.g., C₁-C₄ alkylene, methylene), heteroalkylene (e.g., C₁-C₄ heteroalkylene), —C(O)NR—; —SO₂—, or —C(O)—;
L₃ and L₄ are independently absent, alkylene (e.g., C₁-C₄ alkylene, methylene), or heteroalkylene (e.g., C₁-C₄ heteroalkylene);
R₁ is hydrogen, alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, C₁-C₈ heteroalkyl, C₁-C₅ heteroalkyl, C₃-C₁₂ heterocycloalkyl), halogen (e.g., fluoro, chloro), aryl (e.g., C₆-C₁₂ aryl, phenyl), or heteroaryl (e.g., C₅-C₁₂ heteroaryl, pyridinyl), and R₁ has one or more (e.g., two, three, four, five) optional points of substitution;
R₂ is perfluoroalkyl, aryl (e.g., C₆-C₁₂ aryl, phenyl), arylalkyl (e.g., C₇-C₁₄ alkylaryl, benzyl), alkyl (e.g., C₁-C₁₂ alkyl, C₁-C₈ alkyl, C₁-C₅ alkyl, C₃-C₁₂ cycloalkyl), or heteroaryl (e.g., C₅-C₁₂ heteroaryl, pyridinyl), and R₂ has one or more (e.g., two, three, four, five) optional points of substitution (e.g., with alkoxy, fluoroalkoxy);
R₃ and R₄ are independently hydrogen, —OH, —OR, —S(O)₂R, —N(R)S(O)₂R, —C(O)R, —N(R)C(O) R, —N(R)₂, or heterocyclyl, and R₃ and/or R₄ has one or more (e.g., two, three, four, five) optional points of substitution;
R₅ and R₆ are independently selected from hydrogen and —OH; wherein R₅ and R₆ are not each —OH;
R₇ is hydrogen, —CH₂OH, or —CH₂OR;
R is independently selected at each occurrence from hydrogen and alkyl (e.g., C₁-C₁₂ alkyl, C₁-C₈ alkyl, C₁-C₅ alkyl, C₃-C₁₂ cycloalkyl), wherein each R has one or more (e.g., two, three, four, five) optional points of substitution (e.g., with OH, with C(O)OH, —CN, —NH₂, —N(R_{A})₂); and
R_{A} is independently selected at each occurrence from hydrogen and lower alkyl (e.g., C₁-C₄ alkyl, methyl, ethyl, propyl, isopropyl); or
pharmaceutically acceptable salts thereof; or
prodrugs of any of the foregoing. In various embodiments, the compound does not have the structure:

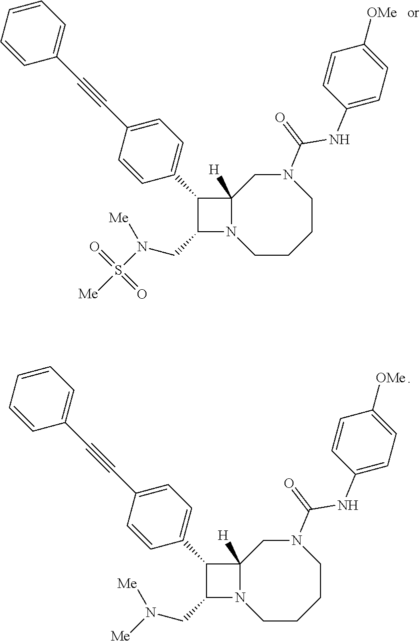 [SMILES: COc1ccc(NC(=O)N2CCCCN3[C@H](CN(C)S(C)(=O)=O)[C@H](c4ccc(C#Cc5ccccc5)cc4)[C@@H]3C2)cc1 ; COc1ccc(NC(=O)N2CCCCN3[C@H](CN(C)C)[C@H](c4ccc(C#Cc5ccccc5)cc4)[C@@H]3C2)cc1]

In some embodiments, R₅ and R₆ are each hydrogen. In various implementations, L₂ is —C(O)NH—. In some embodiments, R₂ is aryl (e.g., phenyl) having one or more (e.g., two, three, four, five) optional points of substitution (e.g., with alkoxy, fluoroalkoxy). For example, R₂ may be para substituted phenyl such as 4-methoxyphenyl. In certain implementations, R₁ is aryl or heteroaryl. In some embodiments, R₁ is optionally substituted phenyl or pyridinyl. In some embodiments, R₇ is —CH₂—OR₈, wherein R₈ is alkyl (e.g., C₁-C₁₂ alkyl, C₁-C₈ alkyl, C₁-C₅ alkyl, C₃-C₁₂ cycloalkyl) having one or more (e.g., two, three, four, five) optional points of substitution (e.g., with OH, with C(O)OH, —CN, —NH₂, —N(R_{A})₂). In some embodiments, R₃ is a group —O(CH₂)ₚC(O)OH or —NH(CH₂)ₚC(O)OH, wherein p is one, two, three, four, or five.

As shown herein, the compound activity is correlated with the structure at various points throughout the compound. In some embodiments, the compound may be characterized in having a % reduction in oocysts/mg of a parasite from the genus Cryptosporidiosium (e.g., C. parvum, C. hominis) 24 hours after its final dose of greater than 25% or greater than 50% or greater than 75% (e.g., as measured in the feces of the immunocompromised mouse model as described herein). In some embodiments, the compound may have a bioavailability (e.g., as determined by pharmacokinetic studies such as those described herein) of more than 25% or more than 50% or more than 75%. In several implementations, the compound may be characterized as having a % reduction in oocysts/mg of a parasite from the genus Cryptosporidiosium (e.g., C. parvum, C. hominis) 24 hours after its final dose of greater than 25% or greater than 50% or greater than 75% (e.g., as measured in the feces of the immunocompromised mouse model as described herein) and a bioavailability (e.g., as determined by pharmacokinetic studies such as those described herein) of more than 25% or more than 50% or more than 75%.

The compound may, for example, have the structure of formula (I):

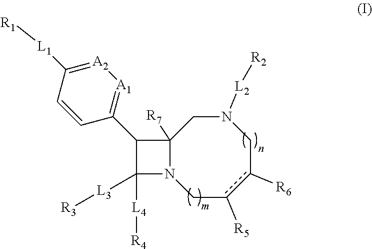

wherein the dashed bond ( [symbol] ) may be a single or double bond;
m is 0 (i.e., it is a bond) or 1;
n is 0, 1 or 2;
A₁ and A₂ are independently CH or N;
L₁ is absent (i.e., it is a bond), or —C≡C—;
L₂ is absent, alkylene (e.g., C₁-C₄ alkylene, methylene), —C(O)NR—; —SO₂—, or —C(O)—;
L₃ and L₄ are independently absent, alkylene (e.g., C₁-C₄ alkylene, methylene), or heteroalkylene (e.g., C₁-C₄ heteroalkylene);
R₁ is hydrogen, alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, C₁-C₈ heteroalkyl, C₁-C₅ heteroalkyl, C₃-C₁₂ heterocycloalkyl), halogen (e.g., fluoro, chloro), aryl (e.g., C₆-C₁₂ aryl, phenyl), heteroaryl (e.g., C₅-C₁₂ heteroaryl, pyridinyl), alkylaryl (e.g., C₇-C₁₄ alkylaryl, tolyl), arylalkyl (e.g., C₇-C₁₄ alkylaryl, benzyl), heteroalkylaryl (e.g., C₇-C₁₄ heteroalkylaryl), heteroarylalkyl (e.g., C₇-C₁₄ heteroarylalkyl), and R₁ has one or more (e.g., two, three, four, five) optional points of substitution;
R₂ is perfluoroalkyl, aryl (e.g., C₆-C₁₂ aryl, phenyl), arylalkyl (e.g., C₇-C₁₄ alkylaryl, benzyl), alkylaryl (e.g., C₇-C₁₄ alkylaryl, tolyl), alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, C₁-C₅ heteroalkyl, C₁-C₅ heteroalkyl, C₃-C₁₂ heterocycloalkyl), or heteroaryl (e.g., C₅-C₁₂ heteroaryl, pyridinyl), and R₂ has one or more (e.g., two, three, four, five) optional points of substitution (e.g., with alkoxy, fluoroalkoxy);
R₃ and R₄ are independently hydrogen, —OH, —OR, —S(O)₂R, —N(R)S(O)₂R, —C(O)R, —N(R)C(O) R, —N(R)₂, or heterocyclyl, and R₃ and/or R₄ has one or more (e.g., two, three, four, five) optional points of substitution;
R₅ and R₆ are independently selected from hydrogen and —OH;
R₇ is hydrogen, —CH₂OH, or —CH₂OR;
R is independently selected at each occurrence from hydrogen and alkyl (e.g., C₁-C₁₂ alkyl, C₁-C₈ alkyl, C₁-C₅ alkyl, C₃-C₁₂ cycloalkyl), wherein each R has one or more (e.g., two, three, four, five) optional points of substitution (e.g., with OH, with C(O)OH, —CN, —NH₂, —N(R_{A})₂); and
R_{A} is independently selected at each occurrence from hydrogen and lower alkyl (e.g., C₁-C₄ alkyl, methyl, ethyl, propyl, isopropyl);
wherein

a) -L₃-R₃ and -L₄-R₄ are each not hydrogen; and/or

b) R₇ is —CH₂OR₈; wherein R₈ is alkyl (e.g., C₁-C₁₂ alkyl, C₁-C₈ alkyl, C₁-C₅ alkyl, C₃-C₁₂ cycloalkyl) having one or more (e.g., two, three, four, five) optional points of substitution (e.g., with OH, with C(O)OH, —CN, —NH₂, —N(R_{A})₂); or

pharmaceutically acceptable salts thereof; or
prodrugs of any of the foregoing. In some embodiments, L₄ is alkylene (e.g., methylene). In some embodiments, one of R₃ or R₄ is —N(R)₂ (e.g., —N(CH₃)₂). In specific implementations at least one of -L₃-R₄ or -L₄-R₄ is dimethylaminomethyl.
In various implementations, the compound has the structure of formula (Ia):

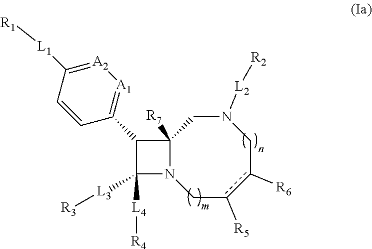

For example, the compound may have the structure of formula (II), (IIa), (III), (IIIa), and/or (IIIb):

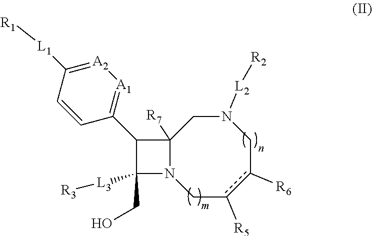

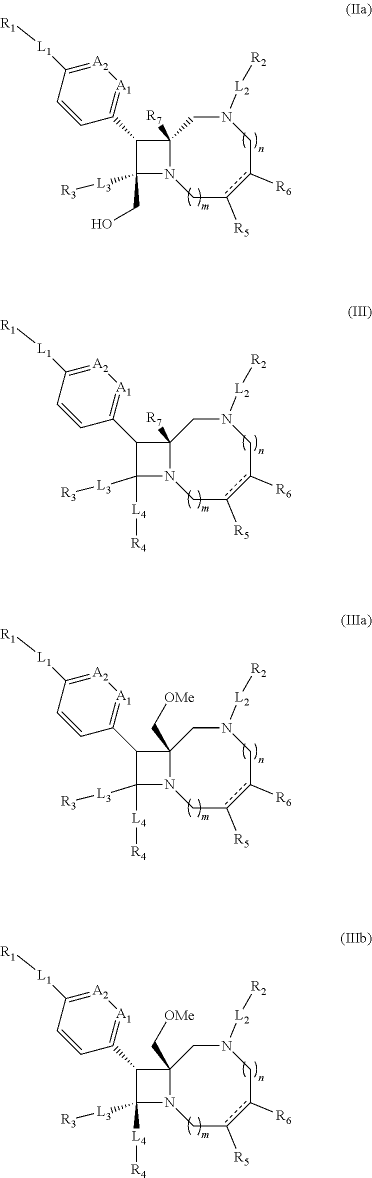

The compound may, for example, have the structure of:

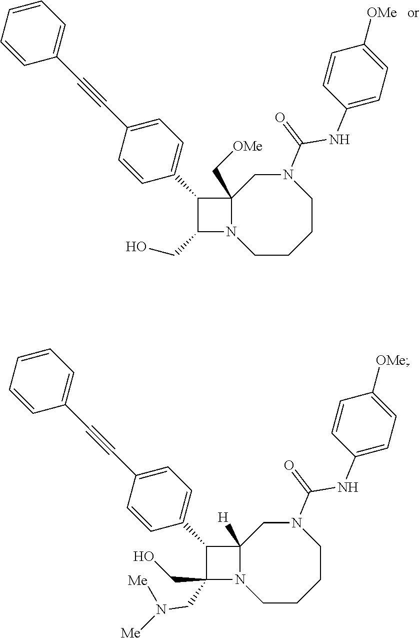 [SMILES: COC[C@@]12CN(C(=O)Nc3ccc(OC)cc3)CCCCN1[C@H](CO)[C@@H]2c1ccc(C#Cc2ccccc2)cc1 ; COc1ccc(NC(=O)N2CCCCN3[C@@H](C2)[C@@H](c2ccc(C#Cc4ccccc4)cc2)[C@@]3(CO)CN(C)C)cc1]

or pharmaceutically acceptable salts thereof; or
prodrugs of any of the foregoing.

In some embodiments, the compounds may be any compound listed in Table 1.

TABLE 1

| Comp | Structure | Name |
|---|---|---|
| 1 | 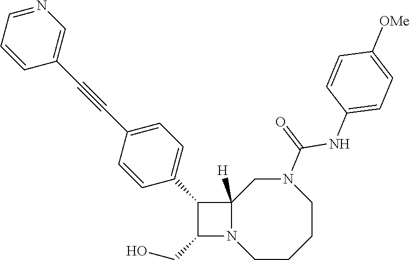 [SMILES: COc1ccc(NC(=O)N2CCCCN3[C@H](CO)[C@H](c4ccc(C#Cc5cccnc5)cc4)[C@@H]3C2)cc1] | (8R,9R,10S)-10-(hydroxymethyl)- N-(4-methoxyphenyl)-9-(4- (pyridin-3-ylethynyl)phenyl)-1,6- diazabicyclo[6.2.0]decane-6- carboxamide |
| 2 | 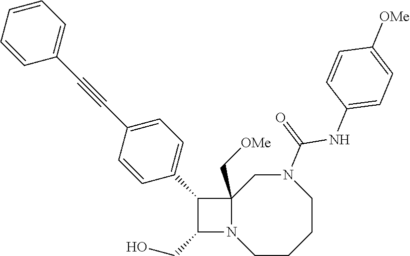 [SMILES: COC[C@@]12CN(C(=O)Nc3ccc(OC)cc3)CCCCN1[C@H](CO)[C@@H]2c1ccc(C#Cc2ccccc2)cc1] | (8R,9S,10S)-10-(hydroxymethyl)- 8-(methoxymethyl)-N-(4- methoxyphenyl)-9-(4- (phenylethynyl)phenyl)-1,6- diazabicyclo[6.2.0]decane-6- carboxamide |
| 3 | 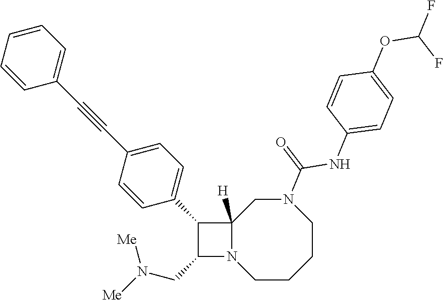 [SMILES: CN(C)C[C@@H]1[C@H](c2ccc(C#Cc3ccccc3)cc2)[C@@H]2CN(C(=O)Nc3ccc(OC(F)F)cc3)CCCCN12] | (8R,9S,10S)-N-(4- (difluoromethoxy)phenyl)-10- ((dimethylamino)methyl)-9-(4- (phenylethynyl)phenyl)-1,6- diazabicyclo[6.2.0]decane-6- carboxamide |
| 4 | 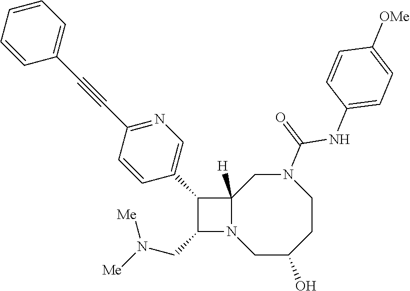 [SMILES: COc1ccc(NC(=O)N2CC[C@H](O)CN3[C@H](CN(C)C)[C@H](c4ccc(C#Cc5ccccc5)nc4)[C@@H]3C2)cc1] | (3S,8R,9S,10S)-10- ((dimethylamino)methyl)-3- hydroxy-N-(4-methoxyphenyl)-9- (6-(phenylethynyl)pyridin-3-yl)- 1,6-diazabicyclo[6.2.0]decane-6- carboxamide |
| 5 | 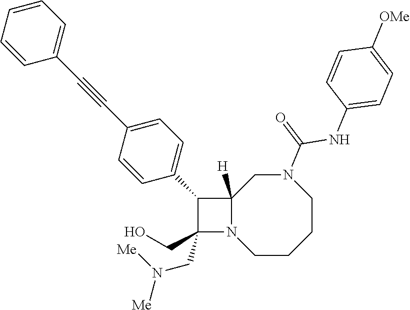 [SMILES: COc1ccc(NC(=O)N2CCCCN3[C@@H](C2)[C@@H](c2ccc(C#Cc4ccccc4)cc2)[C@@]3(CO)CN(C)C)cc1] | (8R,9R,10S)-10- ((dimethylamino)methyl)-10- (hydroxymethyl)-N-(4- methoxyphenyl)-9-(4- (phenylethynyl)phenyl)-1,6- diazabicyclo[6.2.0]decane-6- carboxamide |
| 6 | 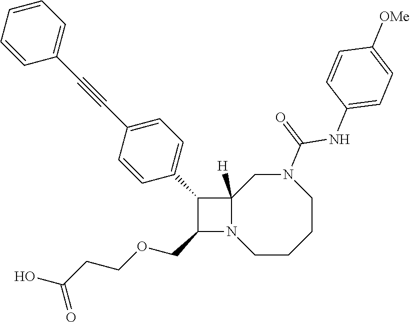 [SMILES: COc1ccc(NC(=O)N2CCCCN3[C@@H](COCCC(=O)O)[C@H](c4ccc(C#Cc5ccccc5)cc4)[C@@H]3C2)cc1] | 3-(((8R,9R,10R)-6-((4- methoxyphenyl)carbamoyl)-9-(4- (phenylethynyl)phenyl)-1,6- diazabicyclo[6.2.0]decan-10- yl)methoxy)propanoic acid |
| 7 | 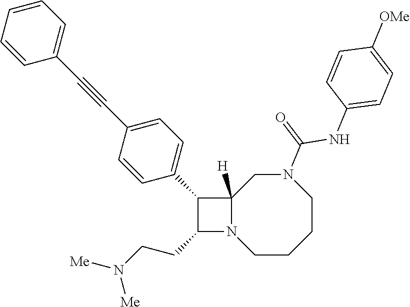 [SMILES: COc1ccc(NC(=O)N2CCCCN3[C@H](CCN(C)C)[C@H](c4ccc(C#Cc5ccccc5)cc4)[C@@H]3C2)cc1] | (8R,9S,10R)-10-(2- (dimethylamino)ethyl)-N-(4- methoxyphenyl)-9-(4- (phenylethynyl)phenyl)-1,6- diazabicyclo[6.2.0]decane-6- carboxamide |
| 8 | 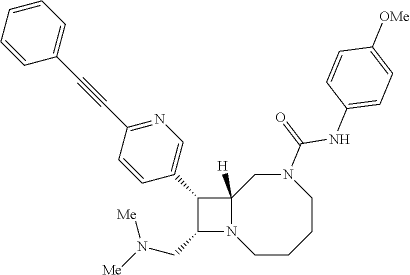 [SMILES: COc1ccc(NC(=O)N2CCCCN3[C@H](CN(C)C)[C@H](c4ccc(C#Cc5ccccc5)nc4)[C@@H]3C2)cc1] | (8R,9S,10S)-10-((dimethylamino) methyl)-N-(4-methoxyphenyl)-9- (6-(phenylethynyl)pyridin-3-yl)- 1,6-diazabicyclo[6.2.0]decane-6- carboxamide |
| 9 | 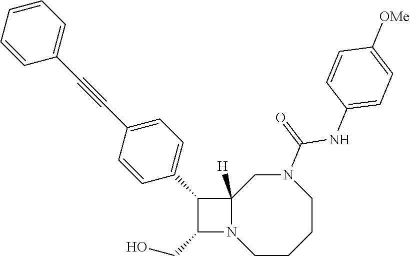 [SMILES: COc1ccc(NC(=O)N2CCCCN3[C@H](CO)[C@H](c4ccc(C#Cc5ccccc5)cc4)[C@@H]3C2)cc1] | (8R,9R,10S)-10-(hydroxymethyl)- N-(4-methoxyphenyl)-9-(4- (phenylethynyl)phenyl)-1,6- diazabicyclo[6.2.0]decane-6- carboxamide |
| 10 | 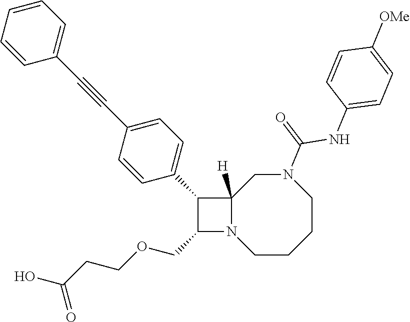 [SMILES: COc1ccc(NC(=O)N2CCCCN3[C@H](COCCC(=O)O)[C@H](c4ccc(C#Cc5ccccc5)cc4)[C@@H]3C2)cc1] | 3-(((8R,9R,10S)-6-((4- methoxyphenyl)carbamoyl)-9-(4- (phenylethynyl)phenyl)-1,6- diazabicyclo[6.2.0]decan-10- yl)methoxy)propanoic acid |
| 11 | 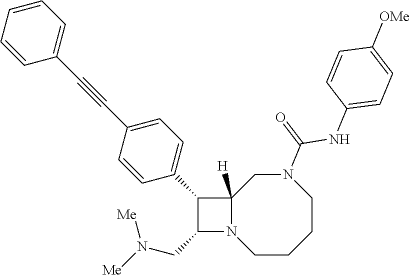 [SMILES: COc1ccc(NC(=O)N2CCCCN3[C@H](CN(C)C)[C@H](c4ccc(C#Cc5ccccc5)cc4)[C@@H]3C2)cc1] | (8R,9S,10S)-10- ((dimethylamino)methyl)-N-(4- methoxyphenyl)-9-(4- (phenylethynyl)phenyl)-1,6- diazabicyclo[6.2.0]decane-6- carboxamide |
| 12 | 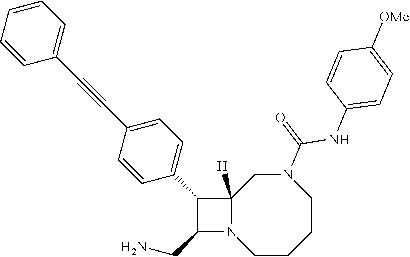 [SMILES: COc1ccc(NC(=O)N2CCCCN3[C@@H](CN)[C@H](c4ccc(C#Cc5ccccc5)cc4)[C@@H]3C2)cc1] | (8R,9S,10R)-10-(aminomethyl)-N- (4-methoxyphenyl)-9-(4- (phenylethynyl)phenyl)-1,6- diazabicyclo[6.2.0]decane-6- carboxamide |
| 13 | 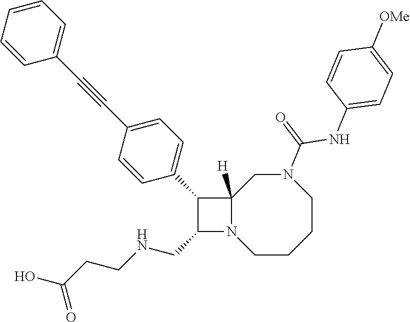 [SMILES: COc1ccc(NC(=O)N2CCCCN3[C@H](CNCCC(=O)O)[C@H](c4ccc(C#Cc5ccccc5)cc4)[C@@H]3C2)cc1] | 3-((((8R,9S,10S)-6-((4- methoxyphenyl)carbamoyl)-9-(4- (phenylethynyl)phenyl)-1,6- diazabicyclo[6.2.0]decan-10- yl)methyl)amino)propanoic acid |
| 14 | 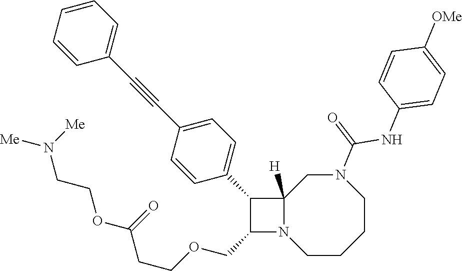 [SMILES: COc1ccc(NC(=O)N2CCCCN3[C@H](COCCC(=O)OCCN(C)C)[C@H](c4ccc(C#Cc5ccccc5)cc4)[C@@H]3C2)cc1] | 2-(dimethylamino)ethyl 3- (((8R,9R,10S)-6-((4- methoxyphenyl)carbamoyl)-9-(4- (phenylethynyl)phenyl)-1,6- diazabicyclo[6.2.0]decan-10- yl)methoxy)propanoate |
| 15 | 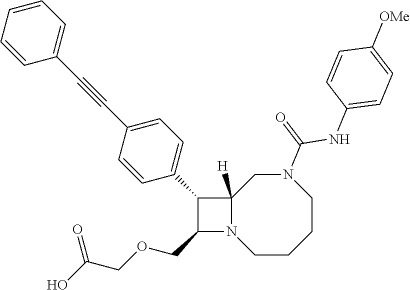 [SMILES: COc1ccc(NC(=O)N2CCCCN3[C@@H](COCC(=O)O)[C@H](c4ccc(C#Cc5ccccc5)cc4)[C@@H]3C2)cc1] | 2-(((8R,9R,10R)-6-((4- methoxyphenyl)carbamoyl)-9-(4- (phenylethynyl)phenyl)-1,6- diazabicyclo[6.2.0]decan-10- yl)methoxy)acetic acid |
| 16 | 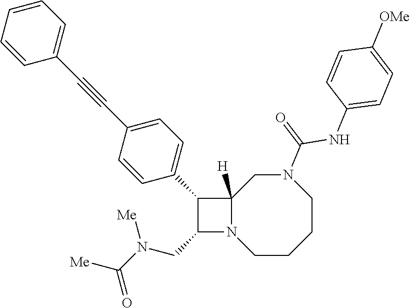 [SMILES: COc1ccc(NC(=O)N2CCCCN3[C@H](CN(C)C(C)=O)[C@H](c4ccc(C#Cc5ccccc5)cc4)[C@@H]3C2)cc1] | (8R,9S,10S)-N-(4-methoxyphenyl)- 10-((N-methylacetamido)methyl)- 9-(4-(phenylethynyl)phenyl)-1,6- diazabicyclo[6.2.0]decane-6- carboxamide |
| 17 | 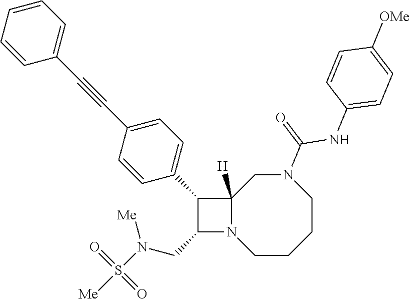 [SMILES: COc1ccc(NC(=O)N2CCCCN3[C@H](CN(C)S(C)(=O)=O)[C@H](c4ccc(C#Cc5ccccc5)cc4)[C@@H]3C2)cc1] | (8R,9R,10S)-N-(4- methoxyphenyl)-10-((N- methylmethylsulfonamido)methyl)- 9-(4-(phenylethynyl)phenyl)-1,6- diazabicyclo[6.2.0]decane-6- carboxamide |
| 18 | 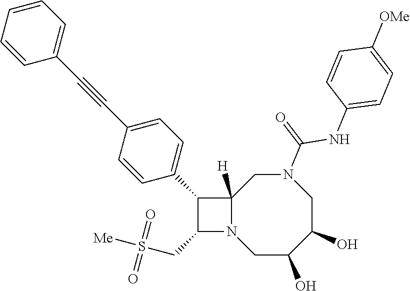 [SMILES: COc1ccc(NC(=O)N2C[C@@H](O)[C@@H](O)CN3[C@H](CS(C)(=O)=O)[C@H](c4ccc(C#Cc5ccccc5)cc4)[C@@H]3C2)cc1] | (3S,4R,8R,9R,10S)-3,4-dihydroxy- N-(4-methoxyphenyl)-10- ((methylsulfonyl)methyl)-9-(4- (phenylethynyl)phenyl)-1,6- diazabicyclo[6.2.0]decane-6- carboxamide |
| 19 | 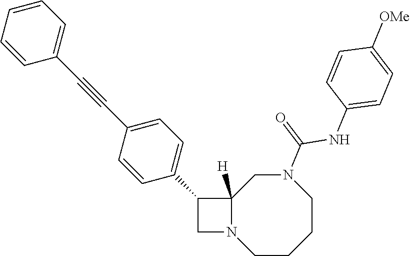 [SMILES: COc1ccc(NC(=O)N2CCCCN3C[C@H](c4ccc(C#Cc5ccccc5)cc4)[C@@H]3C2)cc1] | (8R,9S)-N-(4-methoxyphenyl)-9- (4-(phenylethynyl)phenyl)-1,6- diazabicyclo[6.2.0]decane-6- carboxamide |
| 20 | 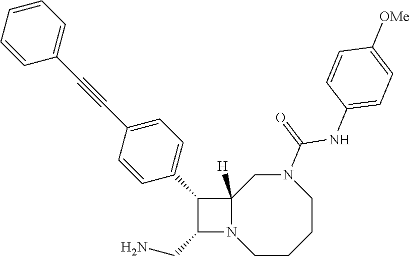 [SMILES: COc1ccc(NC(=O)N2CCCCN3[C@H](CN)[C@H](c4ccc(C#Cc5ccccc5)cc4)[C@@H]3C2)cc1] | (8R,9S,10S)-10-(aminomethyl)-N- (4-methoxyphenyl)-9-(4- (phenylethynyl)phenyl)-1,6- diazabicyclo[6.2.0]decane-6- carboxamide |
| 21 | 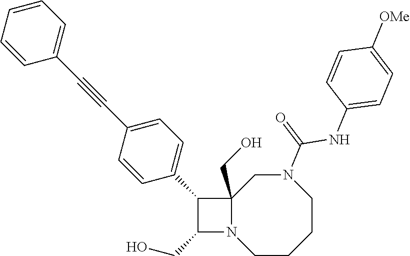 [SMILES: COc1ccc(NC(=O)N2CCCCN3[C@H](CO)[C@H](c4ccc(C#Cc5ccccc5)cc4)[C@]3(CO)C2)cc1] | (8R,9S,10S)-8,10- bis(hydroxymethyl)-N-(4- methoxyphenyl)-9-(4- (phenylethynyl)phenyl)-1,6- diazabicyclo[6.2.0]decane-6- carboxamide |
| 22 | 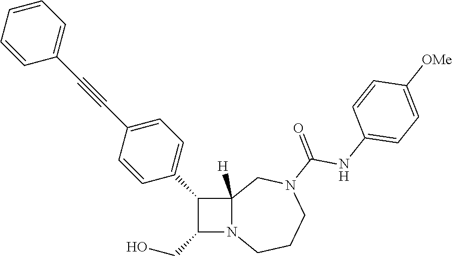 [SMILES: COc1ccc(NC(=O)N2CCCN3[C@H](CO)[C@H](c4ccc(C#Cc5ccccc5)cc4)[C@@H]3C2)cc1] | (7R,8R,9S)-9-(hydroxymethyl)-N- (4-methoxyphenyl)-8-(4- (phenylethynyl)phenyl)-1,5- diazabicyclo[5.2.0]nonane-5- carboxamide |
| 23 | 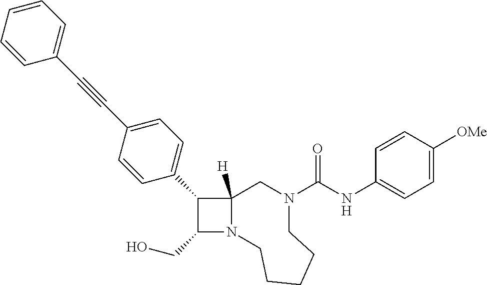 [SMILES: COc1ccc(NC(=O)N2CCCCCN3[C@H](CO)[C@H](c4ccc(C#Cc5ccccc5)cc4)[C@@H]3C2)cc1] | (9R,10R,11S)-11-(hydroxymethyl)- N-(4-methoxyphenyl)-10-(4- (phenylethynyl)phenyl)-1,7- diazabicyclo[7.2.0]undecane-7- carboxamide |
| 24 | 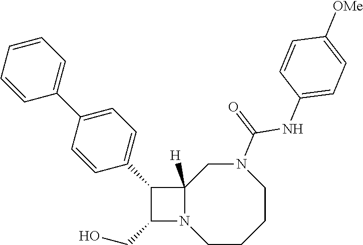 [SMILES: COc1ccc(NC(=O)N2CCCCN3[C@H](CO)[C@H](c4ccc(-c5ccccc5)cc4)[C@@H]3C2)cc1] | (8R,9R,10S)-9-([1,1′-biphenyl]-4- yl)-10-(hydroxymethyl)-N-(4- methoxyphenyl)-1,6- diazabicyclo[6.2.0]decane-6- carboxamide |
| 25 | 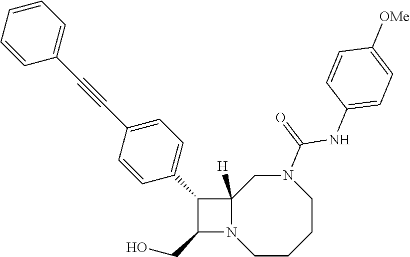 [SMILES: COc1ccc(NC(=O)N2CCCCN3[C@@H](CO)[C@H](c4ccc(C#Cc5ccccc5)cc4)[C@@H]3C2)cc1] | (8R,9R,10R)-10-(hydroxymethyl)- N-(4-methoxyphenyl)-9-(4- (phenylethynyl)phenyl)-1,6- diazabicyclo[6.2.0]decane-6- carboxamide |

It will be understood that in the event of any inconsistency between a chemical name and formula, both compounds with the indicated chemical name and compounds with the indicated chemical structure will be considered as embraced by the invention.

The compounds of the present invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include C₁₋₆ alkyl esters of carboxylic acid groups, which, upon administration to a subject, are capable of providing active compounds.

The compounds described herein (e.g., compounds having the structure of formula (IV), formula (I), formula (II), formula (III)) may be compounds for the treatment of a parasitic disease caused by a parasite from the genus Cryptosporidiosis in a subject in need thereof. The compounds described herein may also be compounds for use in the preparation of a medicament for the treatment of a parasitic disease caused by a parasite from the genus Cryptosporidiosis in a subject in need thereof.

Methods

The compounds described herein are useful in the methods provided herein and, while not bound by any particular theory, are believed to exert their desirable effects through their ability to inhibit the growth of or kill a parasite from the genus Cryptosporidium including cryptosporidiosis. The treatment of cryptosporidiosis may include causative prophylaxis, such as preventing the spread of Cryptosporidium beyond infected portions of a subject (e.g. liver, intestines, respiratory tract).

Methods for the treatment or prophylaxis of a disease caused by parasites from the genus Cryptosporidium are provided comprising administration of one or more compounds (e.g., compounds having the structure of formula (I), (II), (III), (Ia), (IIa), (IIIa), (IIIb), and/or (IV)) to a subject in need thereof. In some embodiments, the composition is formulated in a pharmaceutical composition (e.g., a veterinary composition). The parasitic disease may be cryptosporidiosis. In certain embodiments, the parasite is from the genus of Cryptosporidium, (e.g., C. parvum, C. hominis). The subject may be human. In certain embodiments, the subject is not human (e.g., mouse, rat, rabbit, non-human primate, lizards, geckos, cow, calf, sheep, lamb, horse, foal, pig, piglet). In some embodiments, the subject is administered a therapeutically effective amount of the compound formulated in, for example a pharmaceutical composition.

A method of inhibiting or preventing the growth of a population of parasites from the genus Cryptosporidium in a medium is also provided comprising contacting said population with a compound having the structure of formula (IV) (e.g., compounds having the structure of formula (I), (II), (III), (Ia), (IIa), (IIIa), and/or (IIIb)). In some embodiments, the medium is in vitro (e.g., cell culture medium such as DMEM or fibronectin). In some embodiments, the parasite population has infected a cultured cell. In some embodiments, the medium is in vivo (e.g., in a mouse model, in a human subject). In various embodiments, the Cryptosporidium parasites comprise wild type PheRS. In certain implementations, the Cryptosporidium parasites are C. parvum or C. hominis.

Pharmaceutical Compositions

### 1. Formulations

For use in the methods described herein, the compounds (e.g., compounds having the structure of formula (I), (II), (III), (Ia), (IIa), (IIIa), (IIIb), and/or (IV)) can be formulated as pharmaceutical or veterinary compositions. The formulation selected can vary depending on the subject to be treated, the mode of administration, and the type of treatment desired (e.g., prevention, prophylaxis, or therapy). A summary of formulation techniques is found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference. Exemplary routes of administration and formulations are described as follows.

In the practice of the disclosed methods, the compounds (or pharmaceutically acceptable salts thereof) or compositions can be administered by any of the usual and acceptable routes and methods known in the art. The compounds or compositions can thus be administered, for example, by the enteral or gastrointestinal route (e.g., orally or rectally), topically (e.g., to the skin or an accessible mucous membrane (e.g., an intraoral (e.g., sublingual or buccal), intranasal, intrarectal, or genitourinary surface)), parenterally (e.g., by intramuscular, intravenous, subcutaneous, intraarticular, intravesicular, intrathecal, epidural, ocular, or aural application or injection), transdermally, or by inhalation (e.g., by aerosol).

The compositions can be in the form of a solid, liquid, or gas, as determined to be appropriate by those of skill in the art. Thus, as general examples, the pharmaceutical compositions may be in the form of tablets, capsules, syrups, pills, enterically coated or other protected formulations, sustained release formulations, elixirs, powders, granulates, suspensions, emulsions, solutions, gels (e.g., hydrogels), pastes, ointments, creams, plasters, transdermal patches, drenches, suppositories, enemas, injectables, implants, sprays, or aerosols.

The compositions, in general, include an effective amount of a compound described herein and one or more pharmaceutically acceptable carriers or excipients, as is well known in the art. For example, the compositions can thus include one or more diluents, buffers, preservatives, salts, carbohydrates, amino acids, carrier proteins, fatty acids, or lipids. The compounds described herein may be present in amounts totaling, for example, 1-95% by weight of the total weight of the composition or 1-50% by weight of the total composition or 1-25% by weight of the composition or 1-10% by weight of the composition.

For injection, formulations can be prepared in conventional forms as liquid solutions or suspensions, or as solid forms suitable for solution or suspension in liquid prior to injection, or as emulsions. Suitable excipients for these formulations include, for example, water, saline, dextrose, and glycerol. Such compositions can also contain nontoxic auxiliary substances, such as wetting or emulsifying agents, and pH buffering agents, such as sodium acetate, sorbitan monolaurate, and so forth.

Formulations for oral use include tablets containing a compound in a mixture with one or more non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and anti-adhesives (e.g., magnesium stearate, zinc stearate, stearic acid, silicas, hydrogenated vegetable oils, talc). Other pharmaceutically acceptable excipients can be colorants, flavoring agents, plasticizers, humectants, and buffering agents.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

The pharmaceutical composition may also be formulated as a veterinary composition, intended for use with subjects other than humans. The veterinary compositions according to the present invention can be in any appropriate forms to suit the requested administration modes, for instance nasal, oral, intradermic, cutaneous or parenteral. In a certain embodiment, the composition is in a form intended for an oral administration and, for instance when the domestic animal eating, either mixed to the food ration, or directly into the mouth after meal. The veterinary compositions of the invention are in the form of a nasal, oral or injectable liquid suspension or solution, or in solid or semi-solid form, powders, pellets, capsules, granules, sugar-coated pills, gelules, sprays, cachets, pills, tablets, pastes, implants or gels. In a particular embodiment, the compositions are in the form of an oral solid form including tablets. In some embodiments, the veterinary compositions may have an effective amount of the compound for a specific species of animal (e.g., cow, lamb, goat, horse).

In various embodiments, the compositions of the invention are formulated in pellets or tablets for an oral administration. According to this type of formulation, they comprise lactose monohydrate, cellulose microcrystalline, crospovidone/povidone, aroma, compressible sugar and magnesium stearate as excipients. When the compositions are in the form of pellets or tablets, they are for instance 1 mg, 2 mg, or 4 mg pellets or tablets. Such pellets or tablets are divisible so that they can be cut to suit the posology according to the invention in one or two daily takes. In a further embodiment, the compositions of the disclosure are formulated in injectable solutions or suspensions for a parenteral administration. The injectable compositions are produced by mixing therapeutically efficient quantity of torasemide with a pH regulator, a buffer agent, a suspension agent, a solubilisation agent, a stabilizer, a tonicity agent and/or a preservative, and by transformation of the mixture into an intravenous, sub-cutaneous, intramuscular injection or perfusion according to a conventional method. Possibly, the injectable compositions may be lyophilized according to a conventional method. Examples of suspension agents include methylcellulose, polysorbate 80, hydroxyethylcellulose, xanthan gum, sodic carboxymethylcellulose and polyethoxylated sorbitan monolaurate. Examples of solubilisation agent include polyoxy ethylene-solidified castor oil, polysorbate 80, nicotinamide, polyethoxylated sorbitan monolaurate, macrogol and ethyl ester of caste oil fatty acid. Moreover, the stabilizer includes sodium sulfite, sodium metalsulfite and ether, while the preservative includes methyl p-hydroxybenzoate, ethyl p-hydroxybenzoate, sorbic acid, phenol, cresol and chlorocresol. An example of tonicity agent is mannitol. When preparing injectable suspensions or solutions, it is desirable to make sure that they are blood isotonic.

### 2. Kits

The compounds and compositions can be packaged in a kit, optionally with one or more other pharmaceutical agents. Non-limiting examples of the kits include those that contain, e.g., two or more pills, a pill and a powder, a suppository and a liquid in a vial, or two topical creams. The kits can include optional components that aid in the administration of the unit dose to subjects, such as vials for reconstituting powder forms, syringes for injection, customized IV delivery systems, or inhalers. Additionally, the unit dose kits can contain instructions for preparation and administration of the compositions. The kits can be manufactured as a single use unit dose for one subject, multiple uses for a particular subject (at a constant dose or in which the individual compounds may vary in potency as therapy progresses); or the kits can contain multiple doses suitable for administration to multiple subjects (“bulk packaging”). The kit components can be assembled in cartons, blister packs, bottles, and tubes.

### 3. Dosage

The dose of a compound depends on a number of factors, such as the manner of administration, the age and the body weight of the subject, and the condition of the subject to be treated, and ultimately will be decided by the attending physician or veterinarian. Such an amount of the compound, as determined by the attending physician or veterinarian, is referred to herein, and in the claims, as a “therapeutically effective amount.” For example, the dose of a compound disclosed herein may be in the range of about 1 to about 1000 mg per day. In certain implementations, the therapeutically effective amount is in an amount of from about 1 mg to about 500 mg per day.

Administration of each drug, as described herein, can, independently, be one to four times daily. In some embodiments, administration occurs for a time period ranging from one day to one year, and may even be for the life of the subject. Chronic, long-term administration may be indicated.

### 4. Combination Therapies

The compounds and pharmaceutical compositions can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects).

Examples of other drugs to combine with the compounds described herein include pharmaceuticals for the treatment of cryptosporidiosis (e.g., nitazoxanide, paromomycin, halofuginone). Other examples of drugs to combine with the compounds described herein include pharmaceuticals for the treatment of different, yet associated or related symptoms or indications. Combination methods can involve the use of the two (or more) agents formulated together or separately, as determined to be appropriate by those of skill in the art. In one example, two or more drugs are formulated together for the simultaneous or near simultaneous administration of the agents.

### EXAMPLES

The following examples illustrate specific aspects of the instant description. The examples should not be construed as limiting, as the example merely provides specific understanding and practice of the embodiments and its various aspects.

Synthesis and Characterization of Compounds

General Considerations

Oxygen and/or moisture sensitive reactions were carried out in oven or flame-dried glassware under nitrogen atmosphere. All reagents and solvents were purchased and used as received from commercial vendors or synthesized according to cited procedures. Yields refer to chromatographically and spectroscopically pure compounds, unless otherwise stated. Flash chromatography was performed using 20-40 μm silica gel (60 Å mesh) on a Teledyne Isco Combiflash Rf. Analytical thin layer chromatography (TLC) was performed on 0.2 mm or 0.25 mm silica gel 60-F plates and visualized by UV light (254 nm). NMR spectra were recorded on Bruker 300 (¹H, 300 MHz; ¹³C, 75 MHz) or 400 (¹H, 400 MHz; ¹³C, 100 MHz) or Varian 400MR (¹H, 400 MHz; ¹³C, 100 MHz) spectrometers at 300 K unless otherwise noted. Chemical shifts are reported in parts per million (ppm) relative to the appropriate solvent. Data for ¹H NMR are reported as follows: chemical shift, multiplicity (br=broad, s=singlet, bs=broad singlet, d=doublet, t=triplet, m=multiplet), coupling constants, and integration. Tandem liquid chromatography/mass spectrometry (LCMS) was performed on a Waters 2795 separations module and 3100 mass detector, alternatively a Shimadzu LC-20AD separations module or Agilent 1200 series, with data acquired either directly on reaction mixtures or on purified samples. Preparative HPLC was performed on a Gilson 281 separations module or Shimadzu LC-8A separations module; MS spectra were recorded using a Waters 3100 with electrospray ionization. Samples were eluted using a linear gradient of H₂O/CH₃CN containing one of the following buffers: (A) 0.1% TFA, (B) 0.04% HCl, (C) 0.2% formic acid, (D) 10 mM NH₄HCO₃, or (E) 0.04% NH₄OH.

### Example 1

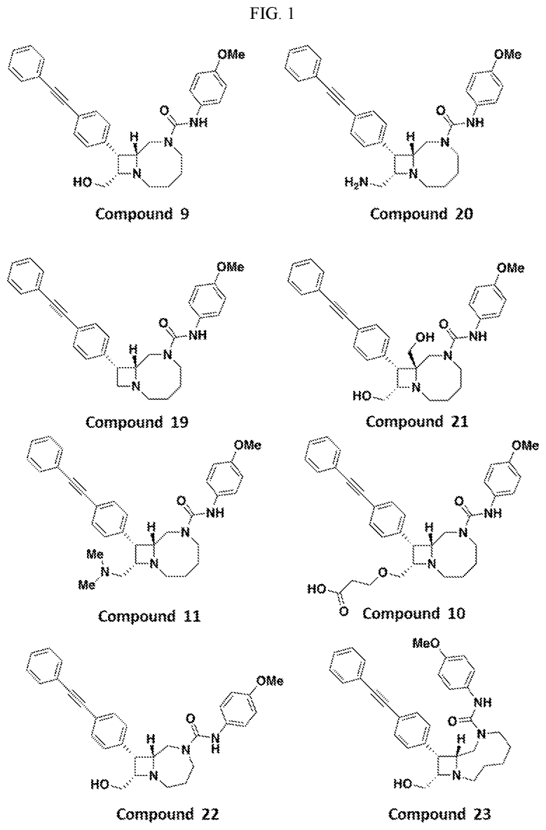

The compounds shown in FIG. 1 were retrieved from the Broad Institute compound management facility as 10 mM DMSO solutions and used without further modification. Analytical data have been reported in the literature, for example in Kato, N. et al. Nature 538, 344-349, doi:10.1038/nature19804 (2016) and Maetani, M. et al. J Am Chem Soc 139, 11300-11306, doi:10.1021/jacs.7b06994 (2017), each of which is hereby incorporated by reference in its entirety and particularly in relation to the synthetic schemes described therein.

### Example 2: Compound 24 ((8R,9R,10S)-9-([1,1′-biphenyl]-4-yl)-10-(hydroxymethyl)-N-(4-methoxyphenyl)-1,6-diazabicyclo[6.2.0]decane-6-carboxamide) characterization

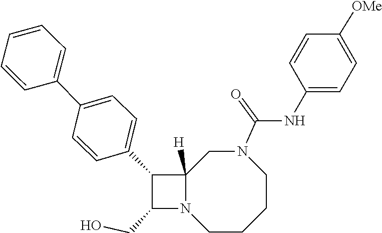 [SMILES: COc1ccc(NC(=O)N2CCCCN3[C@H](CO)[C@H](c4ccc(-c5ccccc5)cc4)[C@@H]3C2)cc1]

LC-MS m/z 471.67 [M+H]⁺

¹H NMR (300 MHz, CDCl₃) δ 7.63-7.56 (m, 2H), 7.54 (s, 4H), 7.48-7.40 (m, 2H), 7.39-7.29 (m, 1H), 7.25 (d, J=8.5 Hz, 2H), 6.82 (d, J=8.9 Hz, 2H), 6.10 (s, 1H), 3.94-3.81 (m, 1H), 3.77 (s, 3H), 3.75-3.58 (m, 5H), 3.56-3.48 (m, 1H), 3.42-3.26 (m, 1H), 3.12-2.89 (m, 2H), 2.47 (dd, J=11.7, 6.9 Hz, 1H), 1.92-1.72 (m, 3H), 1.72-1.56 (m, 2H).

### Example 3: Compound 1 ((8R,9R,10S)-10-(hydroxymethyl)-N-(4-methoxyphenyl)-9-(4-(pyridin-3-ylethynyl)phenyl)-1,6-diazabicyclo[6.2.0]decane-6-carboxamide) characterization

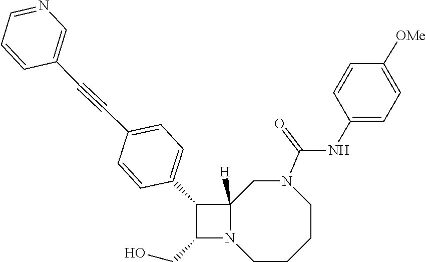 [SMILES: COc1ccc(NC(=O)N2CCCCN3[C@H](CO)[C@H](c4ccc(C#Cc5cccnc5)cc4)[C@@H]3C2)cc1]

LC-MS m/z 497.45 [M+H]⁺

¹H NMR (300 MHz, CDCl₃) δ 8.64 (s, 1H), 8.44 (d, J=4.8 Hz, 1H), 7.73 (d, J=7.9 Hz, 1H), 7.41 (s, 4H), 7.31-7.07 (m, 3H), 6.74 (d, J=8.8 Hz, 2H), 6.10 (s, 1H), 3.95-3.74 (m, 1H), 3.69 (s, 3H), 3.66-3.35 (m, 6H), 3.31-3.15 (m, 1H), 3.05-2.90 (m, 1H), 2.79 (dd, J=14.2, 10.3 Hz, 1H), 2.45-2.29 (m, 1H), 2.03-1.85 (m, 1H), 1.85-1.64 (m, 4H).

### Example 4: Epimers at Azetidine C2 Position

Several compounds (Compound 15, Compound 6, Compound 12) were synthesized from Compound 25 as described in Lowe, J. T. et al. J Org Chem 77, 7187-7211, doi:10.1021/jo300974j (2012) and in Kato, N. et al. Nature 538, 344-349, doi:10.1038/nature19804 (2016), each of which is hereby incorporated by reference in their entirety and particularly in relation to the synthetic schemes described therein.

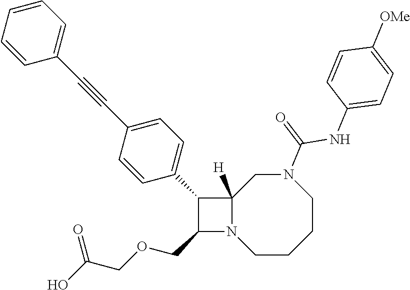 [SMILES: COc1ccc(NC(=O)N2CCCCN3[C@@H](COCC(=O)O)[C@H](c4ccc(C#Cc5ccccc5)cc4)[C@@H]3C2)cc1]

### 2-(((8R,9R,10R)-6-((4-methoxyphenyl)carbamoyl)-9-(4-(phenylethynyl)phenyl)-1,6-diazabicyclo[6.2.0]decan-10-yl)methoxy)acetic acid (Compound 15)

LC-MS m/z 554.33 [M+H]⁺

¹H NMR (400 MHz, CDCl₃) δ 7.61-7.50 (m, 4H), 7.43-7.33 (m, 3H), 7.28 (d, J=4.8 Hz, 4H), 6.91 (br s, 1H), 6.85 (d, J=8.3 Hz, 2H), 4.75 (t, J=10.8 Hz, 1H), 4.27 (d, J=16.9 Hz, 1H), 4.15 (br s, 1H), 4.11-4.03 (m, 2H), 3.98-3.80 (m, 4H), 3.78 (s, 3H), 3.47-3.36 (m, 1H), 3.30-3.07 (m, 2H), 2.97 (dd, J=15.9, 12.2 Hz, 1H), 2.10-1.91 (m, 2H), 1.89-1.72 (m, 2H), 1 exchangeable proton not observed.

### 3-(((8R,9R,10R)-6-((4-methoxyphenyl)carbamoyl)-9-(4-(phenylethynyl)phenyl)-1,6-diazabicyclo[6.2.0]decan-10-yl)methoxy)propanoic acid (Compound 6)

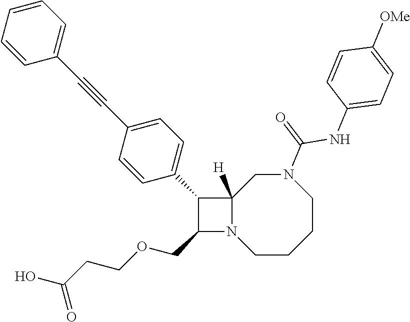 [SMILES: COc1ccc(NC(=O)N2CCCCN3[C@@H](COCCC(=O)O)[C@H](c4ccc(C#Cc5ccccc5)cc4)[C@@H]3C2)cc1]

LC-MS m/z 568.37 [M+H]⁺

¹H NMR (400 MHz, CD₃OD) δ 7.61 (d, J=7.6 Hz, 2H), 7.57-7.50 (m, 2H), 7.46 (d, J=7.6 Hz, 2H), 7.43-7.36 (m, 3H), 7.23 (d, J=8.3 Hz, 2H), 6.87 (d, J=8.3 Hz, 2H), 5.05-4.91 (m, 2H), 4.32 (t, J=9.2 Hz, 1H), 4.06-3.81 (m, 5H), 3.78 (s, 3H), 3.68-3.48 (m, 2H), 3.37 (s, 3H), 2.74-2.64 (m, 2H), 2.09-1.82 (m, 3H), 1.82-1.68 (m, 1H), 2 exchangeable protons not observed.

### (8R,9S,10R)-10-(aminomethyl)-N-(4-methoxyphenyl)-9-(4-(phenylethynyl)phenyl)-1,6-diazabicyclo[6.2.0]decane-6-carboxamide (Compound 12)

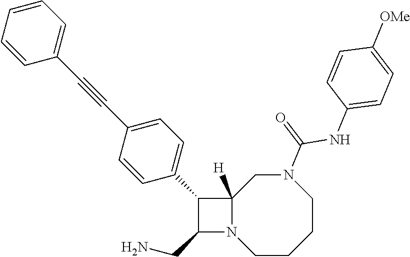 [SMILES: COc1ccc(NC(=O)N2CCCCN3[C@@H](CN)[C@H](c4ccc(C#Cc5ccccc5)cc4)[C@@H]3C2)cc1]

LC-MS m/z 495.35 [M+H]⁺

¹H NMR (400 MHz, CD₃OD) δ 7.55-7.47 (m, 4H), 7.47-7.41 (m, 2H), 7.40-7.31 (m, 3H), 7.19 (d, J=8.3 Hz, 2H), 6.83 (d, J=8.3 Hz, 2H), 4.10 (t, J=9.6 Hz, 1H), 3.90 (dt, J=18.8, 5.6 Hz, 2H), 3.75 (s, 3H), 3.62 (dd, J=8.7, 5.2 Hz, 1H), 3.48 (d, J=14.7 Hz, 1H), 3.25-3.06 (m, 3H), 3.01-2.88 (m, 1H), 2.88-2.73 (m, 2H), 1.87-1.55 (m, 4H), 3 exchangeable protons (NH, NH₂) not observed.

### Example 5: Azetidine C2 Position Analogs

Several compounds were synthesized using Compound 9.

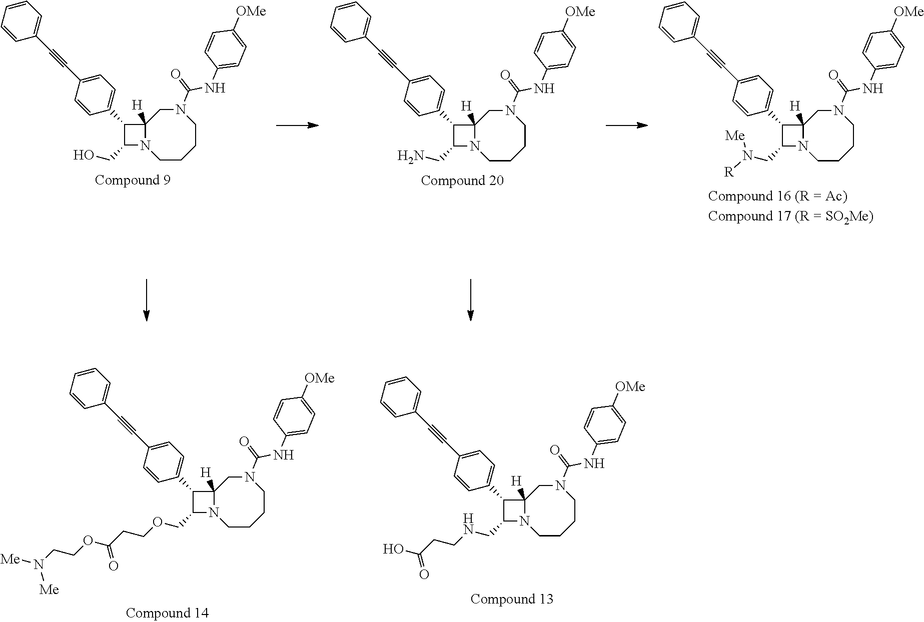

### Synthesis of Compound 13 (3-((((8R,9S,10S)-6-((4-methoxyphenyl)carbamoyl)-9-(4-(phenylethynyl)phenyl)-1,6-diazabicyclo[6.2.0]decan-10-yl)methyl)amino)propanoic acid)

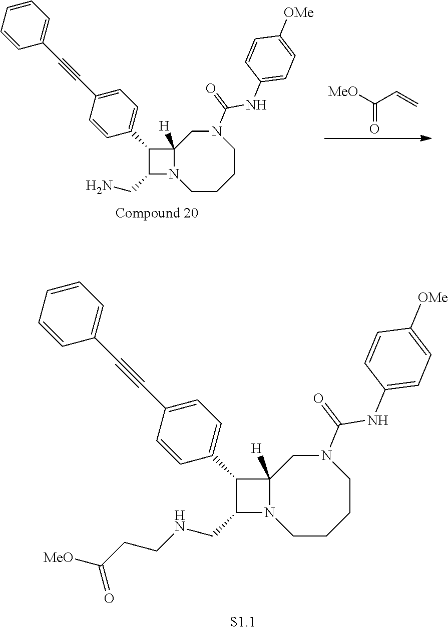

### methyl 3-((((8R,9S,10S)-6-((4-methoxyphenyl)carbamoyl)-9-(4-(phenylethynyl)phenyl)-1,6-diazabicyclo[6.2.0]decan-10-yl)methyl)amino)propanoate (S1.1)

To a solution of the Compound 20 (20 mg, 40 μmol, 1.00 equiv) in dry CH₃OH (600 μL) was added methyl prop-2-enoate (5.6 mg, 66 μmol, 1.6 equiv) at 0° C. under an argon atmosphere. The reaction mixture was warmed slowly to 20° C. and stirred in the dark for 15 h. LC-MS showed the reaction was complete. The reaction mixture was concentrated under reduced pressure. The resulting residue was purified by preparative HPLC (buffer D) to afford the desired compound (15 mg, 26 μmol, 64% yield) as a white solid.

LC-MS m/z 581.1 [M+H]⁺

¹H NMR (400 MHz, MeOD) δ 7.54-7.60 (m, 3H), 7.33-7.42 (m, 3H), 7.47-7.53 (m, 4H), 7.17-7.26 (m, 2H), 6.83 (d, J=9.26 Hz, 2H), 3.98-4.06 (m, 1H), 3.75 (s, 3H), 3.72 (t, J 7.94 Hz, 1H), 3.62 (s, 3H), 3.50-3.60 (m, 2H), 3.39-3.46 (m, 1H), 3.12-3.23 (m, 1H), 2.99-3.10 (m, 2H), 2.67-2.81 (m, 2H), 2.63 (t, J=6.62 Hz, 2H), 2.35-2.40 (m, 1H), 2.29-2.35 (m, 2H), 1.73-1.86 (m, 3H), 1.68-1.87 (m, 1H).

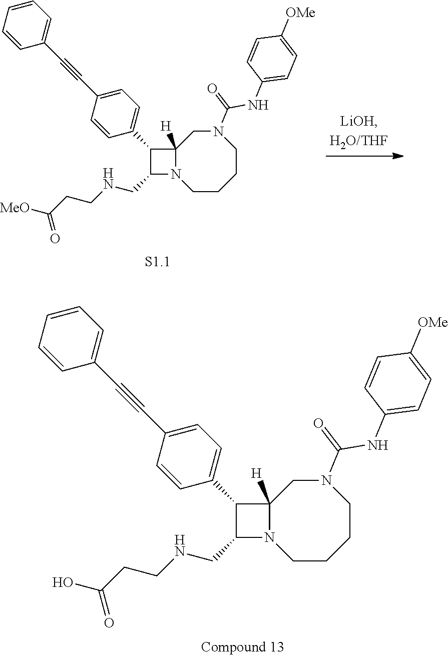 [SMILES: COC(=O)CCNC[C@@H]1[C@H](c2ccc(C#Cc3ccccc3)cc2)[C@@H]2CN(C(=O)Nc3ccc(OC)cc3)CCCCN12 ; COc1ccc(NC(=O)N2CCCCN3[C@H](CNCCC(=O)O)[C@H](c4ccc(C#Cc5ccccc5)cc4)[C@@H]3C2)cc1]

To a solution of S1.1 (9 mg, 16 μmol, 1.0 equiv) in THE (300 μL) was added a solution of LiOH.H₂O (975 μg, 23.25 μmol, 1.5 equiv) in H₂O (300 μL) at 20° C. The reaction mixture was then stirred at 20° C. for 16 h. LC-MS showed the reaction was complete. The reaction mixture was acidified using 1M HCl (5 mL) and concentrated to give a residue. This residue was purified by preparative HPLC (buffer A) to give the desired compound (3 mg, 5.3 μmol, 34% yield) as a white solid.

LC-MS m/z 567.3 [M+H]⁺

¹H NMR (400 MHz, CDCl₃) δ 7.56-7.62 (m, 2H), 7.59-7.48 (m, 5H), 7.32-7.39 (m, 2H), 7.16 (d, J=8.38 Hz, 2H), 6.82 (d, J=8.82 Hz, 2H), 6.33 (br s., 1H), 5.38-5.34 (m, 1H), 4.86-4.82 (m, 1H), 4.26-4.22 (m, 1H), 3.99-3.96 (m, 2H), 3.85-3.82 (m, 2H), 3.76 (s, 3H), 3.48-3.34 (m, 3H), 3.06-3.04 (m, 3H), 2.74-2.70 (m, 2H), 1.77-2.08 (m, 4H).

### Synthesis of Compound 14: 2-(dimethylamino)ethyl 3-(((8R,9R,10S)-6-((4-methoxyphenyl)carbamoyl)-9-(4-(phenyl-ethynyl)phenyl)-1,6-diazabicyclo[6.2.0]decan-10-yl)methoxy)propanoate

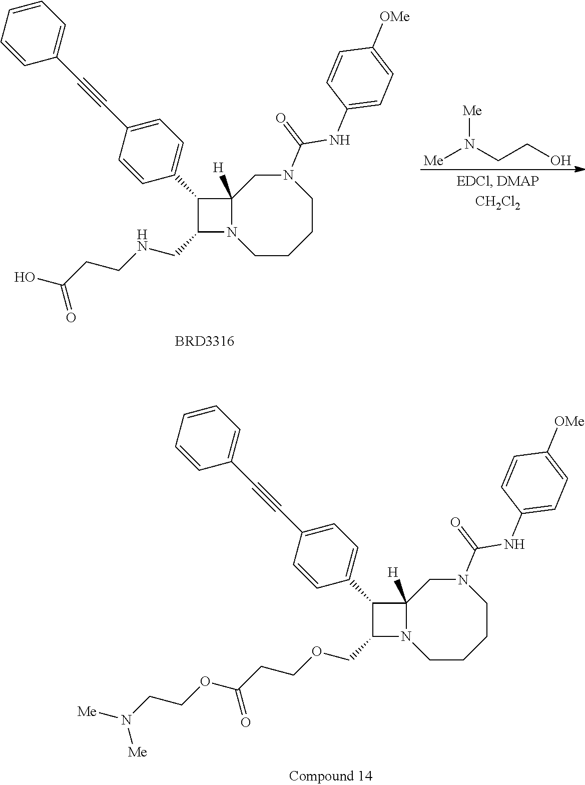 [SMILES: COc1ccc(NC(=O)N2CCCCN3[C@H](CNCCC(=O)O)[C@H](c4ccc(C#Cc5ccccc5)cc4)[C@@H]3C2)cc1 ; CN(C)CCO ; COc1ccc(NC(=O)N2CCCCN3[C@H](COCCC(=O)OCCN(C)C)[C@H](c4ccc(C#Cc5ccccc5)cc4)[C@@H]3C2)cc1]

To a solution of Compound 10 (25 mg, 44 μmol, 1.0 equiv) and 2-(dimethylamino)ethanol (3.6 μL, 57 μmol, 1.3 equiv) in CH₂Cl₂ (0.44 mL) were added EDCI (9.3 mg, 48 μmol, 1.1 equiv) and DMAP (0.5 mg, 4 μmol, 0.1 equiv) in sequence. The resulting solution was stirred overnight at room temperature. LCMS indicated complete conversion. Citric acid (10% aq.) was added, the resulting biphasic mixture was transferred to a separatory funnel, and the layers were separated. The aqueous phase was extracted with CH₂Cl₂ (×3). The combined organic phases were washed with sat. aq. NaHCO₃, dried over MgSO₄, filtered, and concentrated. Flash column chromatography (5-40% EtOAc in hexanes) afforded Compound 14 as a colorless oil.

LC-MS m/z 639.55 [M+H]⁺

¹H NMR (400 MHz, CDCl₃) δ 7.60-7.53 (m, 2H), 7.53-7.43 (m, 4H), 7.42-7.32 (m, 3H), 7.32-7.22 (m, 2H), 6.85 (d, J=8.4 Hz, 2H), 6.10 (s, 1H), 4.32-4.08 (m, 2H), 3.94-3.83 (m, 1H), 3.79 (s, 3H), 3.72-3.41 (m, 6H), 3.41-3.24 (m, 2H), 3.15-3.00 (m, 1H), 2.92 (t, J=12.6 Hz, 1H), 2.69-2.53 (m, 2H), 2.52-2.10 (m, 10H), 1.95-1.56 (m, 4H).

### Synthesis of Compound 17 and Compound 16

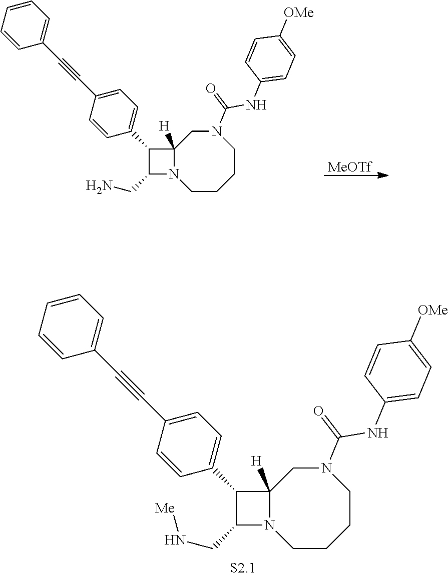 [SMILES: "MeOTf" ; COc1ccc(NC(=O)N2CCCCN3[C@H](CN)[C@H](c4ccc(C#Cc5ccccc5)cc4)[C@@H]3C2)cc1 ; CNC[C@@H]1[C@H](c2ccc(C#Cc3ccccc3)cc2)[C@@H]2CN(C(=O)Nc3ccc(OC)cc3)CCCCN12]

### (8R,9S,10S)—N-(4-methoxyphenyl)-10-((methylamino)methyl)-9-(4-(phenylethynyl)phenyl)-1,6-diazabicyclo[6.2.0]decane-6-carboxamide (S2.1)

To a solution of Compound 20 (210 mg, 425 μmol, 1.00 equiv)¹ in hexafluoroisopropanol (500 μL) was added methyl trifluoromethanesulfonate (105 mg, 637 μmol, 70 μL, 1.5 equiv). The mixture was stirred at 20° C. for 1 h. LC-MS showed that approximately 30% of the starting material remained and a new peak corresponding to the desired compound. The reaction mixture was quenched with NH₄Cl (10 mL), diluted with H₂O (10 mL) and extracted with CH₂Cl₂ (10 mL×5). The combined organic layers were washed with brine (5 mL), dried over anhydrous Na₂SO₄, filtered and concentrated under reduced pressure to give a residue. This residue was purified by preparative TLC (CH₂Cl₂/CH₃OH=10:1) to give the desired compound (140 mg, 275 μmol, 65% yield) as a yellow solid.

LC-MS m/z 509.3 [M+H]⁺

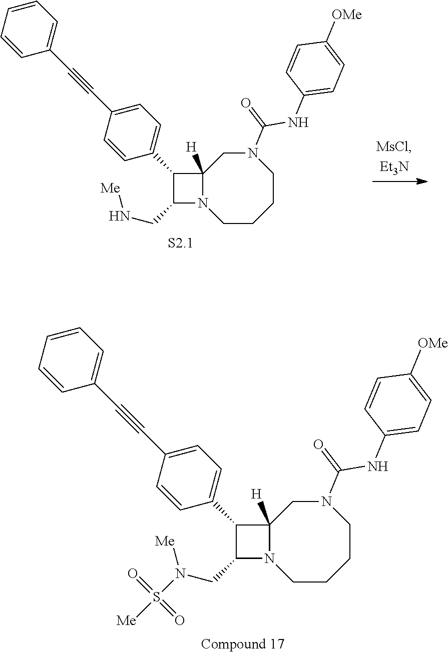 [SMILES: CNC[C@@H]1[C@H](c2ccc(C#Cc3ccccc3)cc2)[C@@H]2CN(C(=O)Nc3ccc(OC)cc3)CCCCN12 ; COc1ccc(NC(=O)N2CCCCN3[C@H](CN(C)S(C)(=O)=O)[C@H](c4ccc(C#Cc5ccccc5)cc4)[C@@H]3C2)cc1]

### (8R,9R,10S)—N-(4-methoxyphenyl)-10-((N-methylmethylsulfonamido)methyl)-9-(4-(phenylethynyl)phenyl)-1,6-diazabicyclo[6.2.0]decane-6-carboxamide (Compound 17)

To a solution of S2.1 (PubChem CID 118072222, 10 mg, 20 μmol, 1.0 equiv) and triethylamine (2 mg, 20 μmol, 3 μL, 1.0 equiv) in CH₂Cl₂ (1 mL) was added methanesulfonyl chloride (3.4 mg, 29 μmol, 3 μL, 1.5 equiv). The mixture was stirred at 20° C. for 12 h. LC-MS showed the reaction was complete. The reaction mixture was concentrated under reduced pressure to remove solvent. This residue was purified by preparative TLC (CH₂Cl₂/CH₃OH=10:1) to give the desired compound (7 mg, 12 μmol, 62% yield) as a yellow solid.

LC-MS m/z 587.2 [M+H]⁺

¹H NMR (400 MHz, CDCl₃) δ 7.55-7.37 (m, 6H), 7.32-7.26 (m, 2H), 7.19-7.12 (m, 3H), 6.76 (d, J=8.8 Hz, 2H), 6.02 (br s, 1H), 3.85-3.74 (m, 1H), 3.70 (s, 3H), 3.66-3.43 (m, 4H), 3.39-3.12 (m, 2H), 3.05 (d, J=7.9 Hz, 1H), 2.92-2.76 (m, 2H), 2.64-2.53 (m, 3H), 2.45 (s, 3H), 2.34 (br. s., 1H), 1.87-1.67 (m, 3H), 1.65-1.57 (m, 1H).

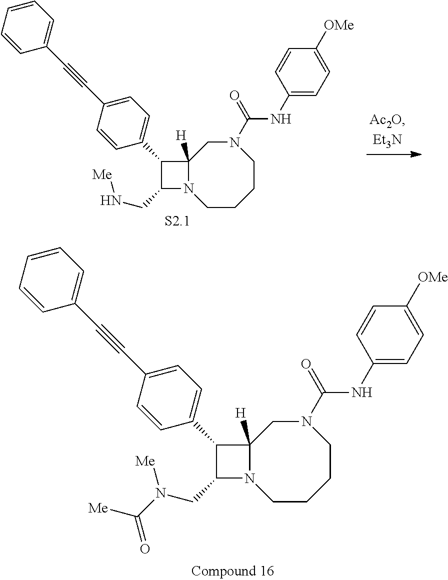 [SMILES: CNC[C@@H]1[C@H](c2ccc(C#Cc3ccccc3)cc2)[C@@H]2CN(C(=O)Nc3ccc(OC)cc3)CCCCN12 ; COc1ccc(NC(=O)N2CCCCN3[C@H](CN(C)C(C)=O)[C@H](c4ccc(C#Cc5ccccc5)cc4)[C@@H]3C2)cc1]

### (8R,9S,10S)—N-(4-methoxyphenyl)-10-((N-methylacetamido)methyl)-9-(4-(phenyl-ethynyl)phenyl)-1,6-diazabicyclo[6.2.0]decane-6-carboxamide (Compound 16)

To a solution of S2.1 (PubChem CID 118072222, 15.0 mg, 29.5 μmol, 1.00 equiv) in CH₂Cl₂ (1.0 mL) was added Ac₂O (4.5 mg, 44 μmol, 4.2 μL, 1.5 equiv). The mixture was stirred at 20° C. for 12 h. LC-MS showed the starting material was consumed completely and one main peak with the desired mass was detected. The reaction mixture was concentrated under reduced pressure. The residue was purified by preparative HPLC (buffer D) to give Compound 16 as a white solid.

LC-MS m/z 551.3 [M+H]⁺

¹H NMR (400 MHz, CDCl₃) δ 7.62-7.47 (m, 6H), 7.38 (d, J=5.0 Hz, 3H), 7.28 (d, J 9.0 Hz, 2H), 6.85 (d, J=6.0 Hz, 2H), 6.17 (d, J=9.0 Hz, 1H), 3.90-3.83 (m, 1H), 3.79 (s, 3H), 3.76-3.42 (m, 5H), 3.27 (d, J=13.1 Hz, 1H), 3.11 (dd, J=5.0, 14.1 Hz, 1H), 3.03-2.84 (m, 2H), 2.76 (s, 1H), 2.59 (s, 2H), 2.45-2.32 (m, 1H), 2.02 (d, J=5.0 Hz, 3H), 1.88-1.74 (m, 3H), 1.72-1.59 (m, 1H).

### Example 6: Compound 7 ((8R,9S,10R)-10-(2-(dimethylamino)ethyl)-N-(4-methoxyphenyl)-9-(4-(phenylethynyl)phenyl)-1,6-diazabicyclo[6.2.0]decane-6-carboxamide)

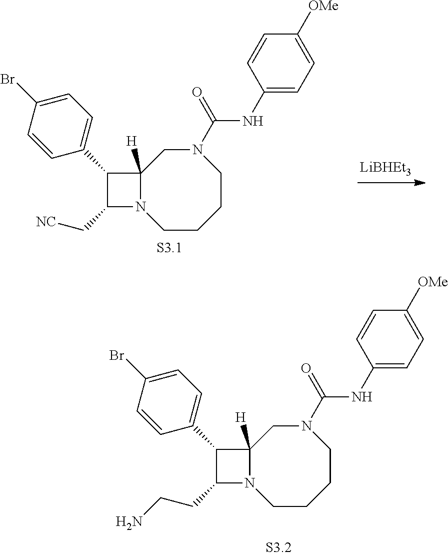 [SMILES: COc1ccc(NC(=O)N2CCCCN3[C@H](CC#N)[C@H](c4ccc(Br)cc4)[C@@H]3C2)cc1 ; COc1ccc(NC(=O)N2CCCCN3[C@H](CCN)[C@H](c4ccc(Br)cc4)[C@@H]3C2)cc1]

### (8R,9S,10R)-10-(2-aminoethyl)-9-(4-bromophenyl)-N-(4-methoxyphenyl)-1,6-diazabicyclo[6.2.0]decane-6-carboxamide (S3.2)

To a precooled (0° C.) solution of S3.1 (250 mg, 517 μmol, 1.00 equiv) in THE (20 mL) was added LiBHEt₃ (1 M in THF, 5.17 mL, 10 equiv). The mixture was warmed to rt and stirred for 16 h. TLC (CH₂Cl₂/CH₃OH=20:1) showed that the substrate was consumed completely and a new spot was detected. The reaction mixture was quenched by addition of sat. aq. NH₄Cl (10 mL), and extracted with EtOAc (5 mL×2). Organic layers were combined, washed with brine (5 mL×2), dried over Na₂SO₄, filtered and concentrated under reduced pressure. Compound S3.2 (600 mg, crude) was used in the next step without further purification.

LC-MS m/z 487.2 [M+H]⁺

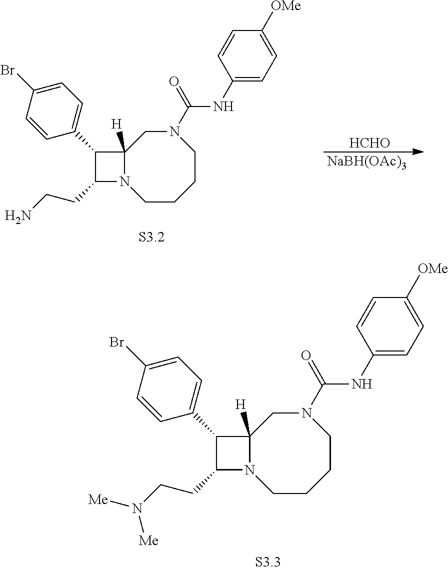 [SMILES: COc1ccc(NC(=O)N2CCCCN3[C@H](CCN)[C@H](c4ccc(Br)cc4)[C@@H]3C2)cc1 ; "HCHO" ; COc1ccc(NC(=O)N2CCCCN3[C@H](CCN(C)C)[C@H](c4ccc(Br)cc4)[C@@H]3C2)cc1 ; "NaBH(OAc)3"]

### (8R,9S,10R)-9-(4-bromophenyl)-10-(2-(dimethylamino)ethyl)-N-(4-methoxyphenyl)-1,6-diazabicyclo[6.2.0]decane-6-carboxamide (S3.3)

To a solution of S3.2 (165 mg, 339 μmol, 1.00 equiv) in CH₂Cl₂ (15.0 mL) were added formalin (formaldehyde 37% w/w in H₂O, 165 mg, 2.03 mmol, 6.0 equiv) and NaBH(OAc)₃ (1.00 g, 4.74 mmol, 14.0 equiv). The mixture was stirred at room temperature for 16 h. LC-MS showed that the substrate was consumed completely and one main peak with the desired mass was detected. The reaction mixture was partitioned between sat. aq. NaHCO₃ (10 mL) and EtOAc (10 mL). The organic phase was separated, and the aqueous phase washed with EtOAc (5 mL×3). The combined organic layers were washed with brine (10 mL), dried over Na₂SO₄, filtered and concentrated under reduced pressure. The resulting residue was purified by preparative HPLC (buffer D) to afford S3.3 (60.0 mg, 116 μmol, 34% yield) as a white solid.

LC-MS m/z 515.1 [M+H]⁺

¹H NMR (400 MHz, CDCl₃) δ 7.40-7.26 (m, 4H), 7.23-7.09 (m, 1H), 6.80-6.67 (m, 2H), 6.15-5.97 (m, 1H), 3.77 (br d, J=15.4 Hz, 1H), 3.70 (s, 3H), 3.54 (br d, J=14.2 Hz, 1H), 3.40 (app. s, 2H), 3.17 (app. s, 2H), 2.95-2.83 (m, 1H), 2.75 (m, 1H), 2.24-2.14 (m, 1H), 2.07-1.89 (m, 7H), 1.79-1.63 (m, 4H), 1.65-1.48 (m, 3H).

### (8R,9S,10R)-10-(2-(dimethylamino)ethyl)-N-(4-methoxyphenyl)-9-(4-(phenylethynyl)phenyl)-1,6-diazabicyclo[6.2.0]decane-6-carboxamide (Compound 7)

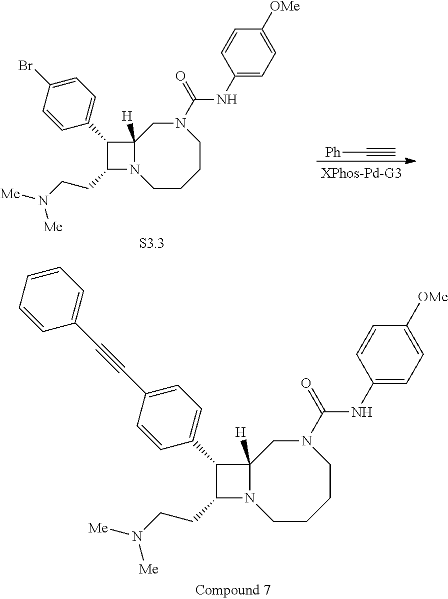

To a solution of S3.3 (65.0 mg, 126 μmol, 1.00 equiv) in CH₃CN (1.00 mL) was added phenylacetylene (39 mg, 0.38 mmol, 3.0 equiv), Cs₂CO₃ (164 mg, 504 μmol, 4.00 equiv) and XPhos-Pd-G3 (10.7 mg, 12.6 μmol, 0.10 equiv). The mixture was stirred at 70° C. for 16 h. The reaction mixture was partitioned between H₂O (10 mL) and EtOAc (10 mL). The organic phase was separated, the aqueous phase washed with EtOAc (5 mL×3). The combined organic layers were washed with brine (10 mL), dried over Na₂SO₄, filtered and concentrated under reduced. LC-MS showed that the substrate had been consumed completely and one main peak with the desired mass was detected. The residue was purified by preparative TLC (CH₂Cl₂/CH₃OH=10:1) to afford Compound 7 (23.0 mg, 42.9 μmol, 34% yield) as a gray oil.

LC-MS m/z 537.4 [M+H]⁺

¹H NMR (400 MHz, CD₃OD) δ 7.59-7.52 (m, 2H), 7.52-7.45 (m, 4H), 7.41-7.30 (m, 3H), 7.24-7.15 (m, 2H), 6.86-6.77 (m, 2H), 4.07-3.92 (m, 1H), 3.73 (s, 3H), 3.64-3.42 (m, 3H), 3.20-3.19 (m, 1H), 3.18-3.08 (m, 1H), 3.06-2.92 (m, 2H), 2.40-2.15 (m, 2H), 2.09-1.96 (m, 6H), 1.89-1.59 (m, 7H), 1 exchangeable proton not observed.

### Example 7: Modification of Urea Appendage (e.g., Compound 3)

### (8R,9S,10S)—N-(4-(difluoromethoxy)phenyl)-10-((dimethylamino)methyl)-9-(4-(phenylethynyl)phenyl)-1,6-diazabicyclo[6.2.0]decane-6-carboxamide (Compound 3)

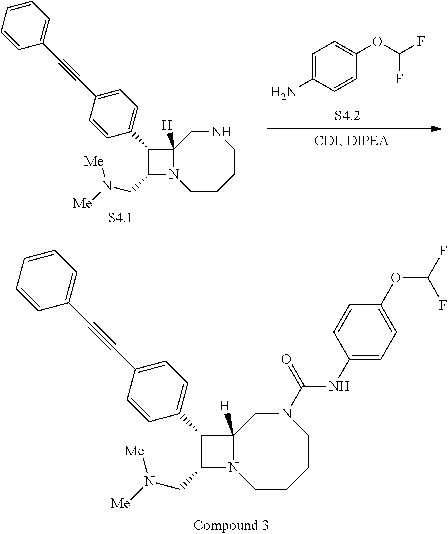 [SMILES: CN(C)C[C@@H]1[C@H](c2ccc(C#Cc3ccccc3)cc2)[C@@H]2CNCCCCN12 ; Nc1ccc(OC(F)F)cc1 ; CN(C)C[C@@H]1[C@H](c2ccc(C#Cc3ccccc3)cc2)[C@@H]2CN(C(=O)Nc3ccc(OC(F)F)cc3)CCCCN12]

To a stirred solution of S4.2 (8.5 mg, 54 μmol, 6.7 μL, 1.0 equiv) in DMF (0.12 mL) were added DIPEA (7.4 mg, 10 μL, 57 μmol, 1.1 equiv) and CDI (8.7 mg, 54 μmol, 1.0 equiv). The reaction mixture was stirred at rt for 30 minutes. To this mixture was added a solution of S4.1 (20.0 mg, 53.5 μmol, 1.00 equiv-prepared in analogous fashion to S5.12, see, e.g., synthesis of Compound 8) and DIPEA (7.4 mg, 10 μL, 57 μmol, 1.1 equiv) in DMF (0.12 mL). The reaction mixture was stirred at rt for an additional 16 h. LCMS showed the substrate was consumed completely. The reaction mixture was dissolved in H₂O (2 mL) and extracted with CH₂Cl₂ (3 mL×3). The organic layer was dried over Na₂SO₄ and concentrated under reduced pressure. The residue was purified by preparative TLC (CH₂Cl₂/CH₃OH=10:1) followed by preparative HPLC (buffer C) to afford Compound 3 (formate salt, 9.50 mg, 15.7 μmol, 29% yield) as a white solid.

LC-MS m/z 559.3 [M+H]⁺

¹H NMR (400 MHz, CDCl₃) δ 7.57-7.44 (m, 6H), 7.40-7.32 (m, 5H), 7.05 (d, J=9.04 Hz, 2H), 6.44 (s, 1H), 6.24 (s, 1H), 3.87 (br d, J=14.99 Hz, 1H), 3.71-3.50 (m, 4H), 3.36-3.21 (m, 1H), 3.13-3.01 (m, 1H), 2.98-2.82 (m, 1H), 2.55 (br d, J=5.51 Hz, 2H), 2.42-2.29 (m, 1H), 2.10 (s, 6H), 1.95-1.58 (m, 4H).

### Example 8: Modification of Proximal Aromatic Ring and Bicyclic Azetidine Core (e.g., Compound 8, Compound 4, Compound 18, Compound 5, Compound 2)

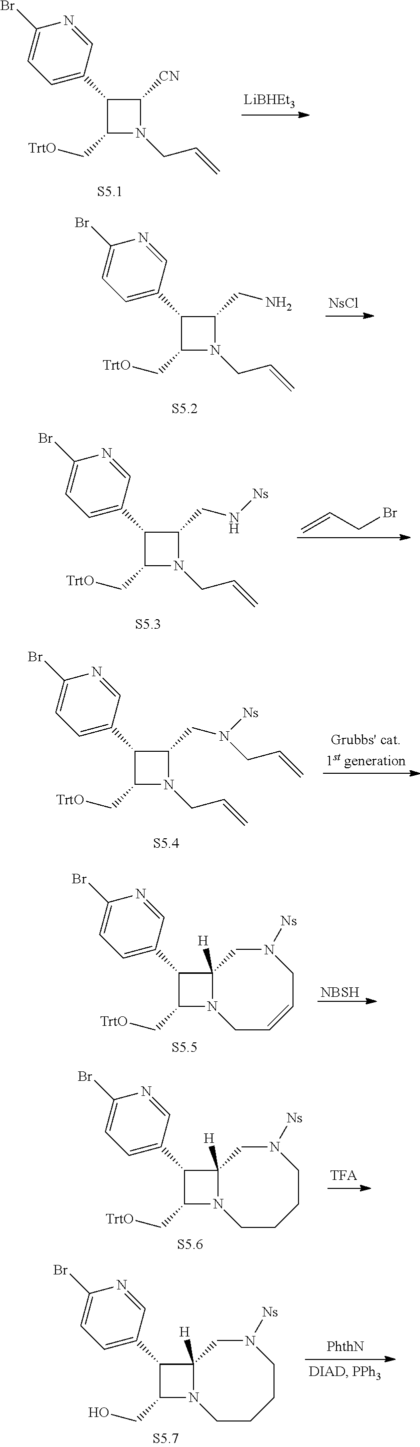

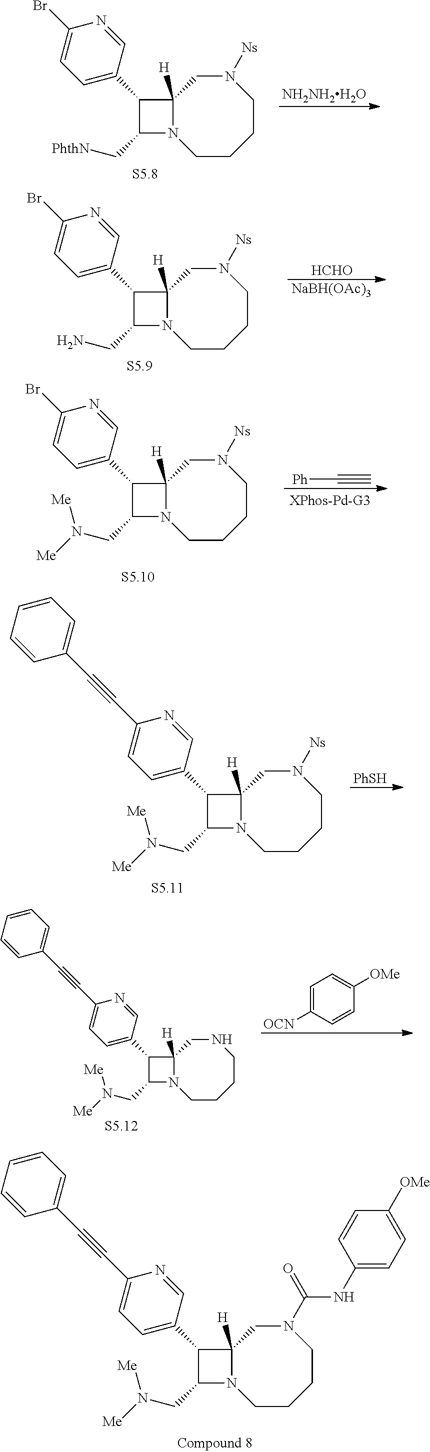

### ((2R,3R,4S)-1-allyl-3-(6-bromopyridin-3-yl)-4-((trityloxy)methyl)azetidin-2-yl) methanamine (S5.2)

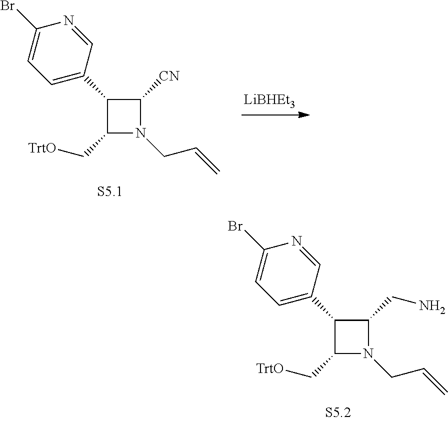

To a solution of S5.1 (3.6 g, 6.54 mmol, 1.0 equiv) in THE (100 mL) was added lithium triethylborohydride (1 M solution in THF, 39.2 mL, 39 mmol, 6.54 mmol, 6.0 equiv) at 0° C. LC-MS showed the reaction was completed. To the reaction mixture was slowly added sat. aq. NH₄Cl (50 mL) and EtOAc (20 mL). The organic phase was separated, and the aqueous phase was extracted with EtOAc (20 mL×3). The combined organic phases were washed with brine (50 ml), dried over Na₂SO₄, filtered and concentrated under reduced pressure to give a residue which was purified by column chromatography (SiO₂, CH₂Cl₂/CH₃OH=1:0 to 10:1) to afford the desired compound (4.18 g, 5.7 mmol, 87% yield, 75% purity) as a red brown solid that was used directly in the subsequent step.

LC-MS m/z 578.1 (base peak) [M+H]⁺

### N-(((2R,3R,4S)-1-allyl-3-(6-bromopyridin-3-yl)-4-((trityloxy)methyl)azetidin-2-yl)methyl)-4-nitrobenzenesulfonamide (S5.3)

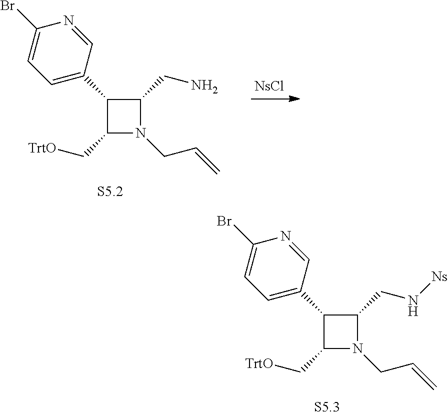

To a solution of S5.2 (4.18 g crude, assumed 5.68 mmol, 1.00 equiv) in CH₂Cl₂ (60 mL) was added 2,6-lutidine (1.83 g, 17.0 mmol, 1.98 mL, 3.0 equiv) and 2-nitrobenzenesulfonyl chloride (1.38 g, 6.25 mmol, 1.10 equiv) at 0° C. The resulting solution was stirred at 25° C. for 13 h. LC-MS showed the reaction was complete. The reaction mixture was quenched with H₂O (50 mL) at 25° C., and extracted with CH₂Cl₂ (30 mL×3). The combined organic layers were washed with brine (50 mL), dried over Na₂SO₄, filtered and concentrated under reduced pressure. The residue was purified by column chromatography (SiO₂, petroleum ether/EtOAc=10:1 to 3:1) to give the desired compound (1.50 g, 2.03 mmol, 36% yield) as a red brown solid.

LC-MS m/z 739.1 [M+H]⁺

¹H NMR (400 MHz, CDCl₃) δ 8.15 (d, J=2.4 Hz, 1H), 7.74-7.70 (m, 2H), 7.71-7.67 (m, 1H), 7.48-7.40 (m, 1H), 7.20-7.17 (m, 16H), 7.09 (d, J=2.2 Hz, 1H), 5.72-5.53 (m, 1H), 5.14 (m, 1H), 5.05-4.94 (m, 1H), 4.95-4.83 (m, 1H), 3.73-3.62 (m, 2H), 3.61-3.51 (m, 1H), 3.20-3.06 (m, 4H), 2.80-2.71 (m, 1H), 2.66 (m, 1H).

### N-allyl-N-(((2R,3R,4S)-1-allyl-3-(6-bromopyridin-3-yl)-4-((trityloxy)methyl)azetidin-2-yl)methyl)-4-nitrobenzenesulfonamide (S5.4)

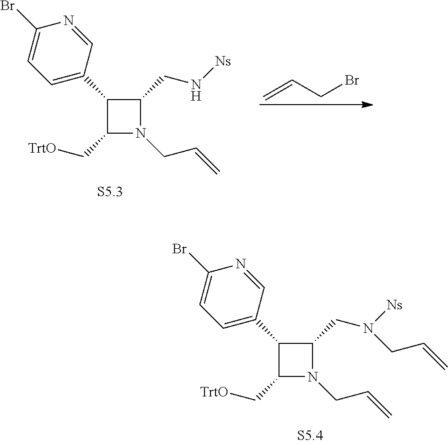

To a solution of S5.3 (1.50 g, 2.03 mmol, 1.0 equiv) in DMF (8 mL) were allyl bromide (270 mg, 2.23 mmol, 1.1 equiv) and potassium carbonate (421 mg, 3.04 mmol, 1.5 equiv) at 0° C. The temperature was increased gradually, with constant stirring, to 30° C. and the reaction was stirred for an additional 13 h. LC-MS showed the reaction was completed. To the reaction mixture was added H₂O (20 mL) and the resulting mixture was extracted with EtOAc (30 mL×3). The organic layer was washed with brine, dried over sodium sulfate and concentrated to give a residue. The residue was purified by column chromatography (SiO₂, petroleum ether/EtOAc=20:1 to 3:1) to give the desired (1.40 g, 1.80 mmol, 89% yield) as a red brown oil.

LC-MS m/z 781.0 (base peak) [M+H]⁺

¹H NMR (400 MHz, CDCl₃) δ 8.20 (d, J=2.4 Hz, 1H), 7.73-7.47 (m, 5H), 7.21-7.11 (m, 16H), 5.70-5.61 (m, 1H), 5.53-5.39 (m, 1H), 5.22-4.83 (m, 4H), 3.87-3.70 (m, 2H), 3.66-3.51 (m, 3H), 3.31-2.98 (m, 5H), 2.65 (t, J=9.3 Hz, 1H).

### (8R,9R,10S,Z)-9-(6-bromopyridin-3-yl)-6-((4-nitrophenyl)sulfonyl)-10-((trityloxy)methyl)-1,6-diazabicyclo[6.2.0]dec-3-ene (S5.5)

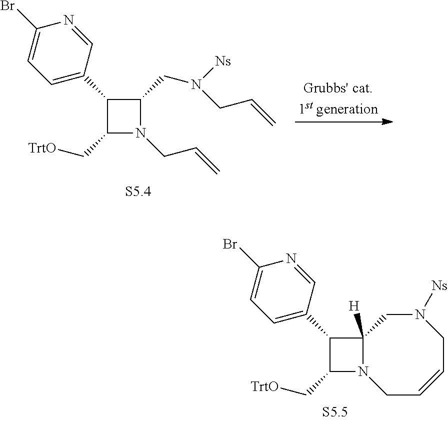

To a solution of S5.4 (1.30 g, 1.67 mmol, 1.0 equiv) in toluene (100 mL) was added Grubbs catalyst 1^{st} Generation (343 mg, 418 μmol, 0.25 equiv) and the solution was stirred at 50° C. for 16 h. LC-MS showed the reaction was completed. The reaction mixture was quenched with H₂O (20 mL), and then extracted with CH₂Cl₂ (10 mL×3). The combined organic layers were dried over Na₂SO₄, filtered and concentrated under reduced pressure to give a residue. The residue was purified by column chromatography on silica gel (petroleum ether/EtOAc=10:1 to 2:1) to give the desired compound (762 mg, 1.01 mmol, 61% yield) as a red brown solid.

LC-MS m/z 751.1 [M+H]⁺

¹H NMR (400 MHz, CDCl₃) δ 8.20 (d, J=2.3 Hz, 1H), 7.90 (dd, J=1.6, 7.6 Hz, 1H), 7.72-7.62 (m, 2H), 7.61-7.57 (m, 1H), 7.53-7.49 (m, 1H), 7.26-7.14 (m, 16H), 5.90-5.84 (m, 1H), 5.75-5.64 (m, 1H), 4.09-4.01 (m, 1H), 3.99-3.90 (m, 1H), 3.73-3.57 (m, 3H), 3.44 (dd, J=6.6, 14.9 Hz, 1H), 3.28-3.10 (m, 3H), 3.03-2.96 (m, 1H), 2.81-2.76 (m, 1H).

### (8R,9R,10S)-9-(6-bromopyridin-3-yl)-6-((4-nitrophenyl)sulfonyl)-10-((trityloxy)methyl)-1,6-diazabicyclo[6.2.0]decane (S5.6)

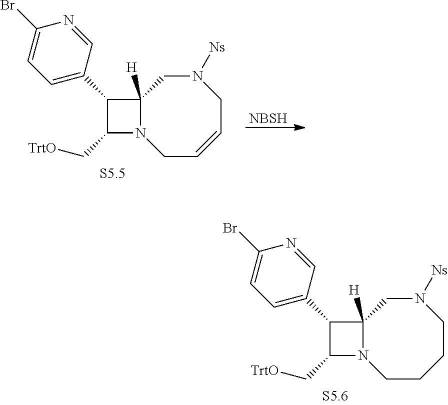

To a solution of S5.5 (760 mg, 1.0 mmol, 1.0 equiv) in THE (20 mL) was added 2-nitrobenzenesulfonohydrazide (658 mg, 3.03 mmol, 3.0 equiv) and triethylamine (920 mg 9.1 mmol, 1.3 mL, 9.0 equiv) at 25° C. The mixture was stirred at 40° C. for 16 h. LC-MS showed the reaction was completed. The reaction mixture was quenched with H₂O (10 mL), and then extracted with CH₂Cl₂ (10 mL×3). The combined organic layers were dried over Na₂SO₄, filtered and concentrated under reduced pressure to give a residue. The residue was purified by column chromatography (SiO₂, petroleum ether/EtOAc=10:1 to 2:1) to give the desired compound (680 mg, 902 μmol, 89% yield) as an off-white solid.

LC-MS m/z 753.1 [M+H]⁺

¹H NMR (400 MHz, CDCl₃) δ 8.25 (d, J=2.3 Hz, 1H), 7.79-7.76 (m, 1H), 7.65-7.61 (m, 2H), 7.57-7.53 (m, 1H), 7.51-7.48 (m, 1H), 7.26-7.16 (m, 16H), 3.87-3.76 (m, 1H), 3.73-3.60 (m, 3H), 3.26-3.16 (m, 2H), 3.11-2.91 (m, 2H), 2.86-2.68 (m, 2H), 2.53-2.43 (m, 1H), 1.95-1.77 (m, 3H), 1.68-1.58 (m, 1H).

### ((8R,9R,10S)-9-(6-bromopyridin-3-yl)-6-((4-nitrophenyl)sulfonyl)-1,6-diazabicyclo[6.2.0]decan-10-yl)methanol (S5.7)

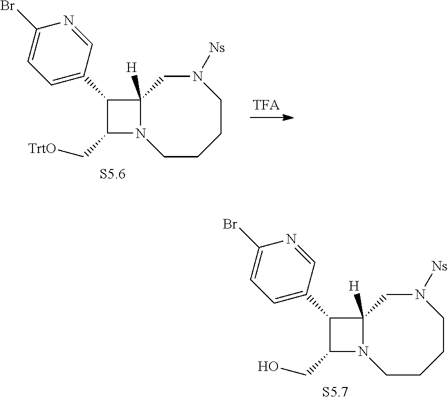

To a solution of S5.6 (680 mg, 902 μmol, 1.0 equiv) in CH₂Cl₂ (15 mL) was added TFA (1.03 g, 9.02 mmol, 692 μL, 10.0 equiv). The mixture was stirred at 20° C. for 16 h. LC-MS showed the reaction was completed. The mixture was adjusted to pH 9 with sat. aq. NaHCO₃ and then extracted with EtOAc (30 mL×3). The organic components were separated, washed with brine (10 mL×2), dried over Na₂SO₄, filtered and concentrated under reduced pressure. The residue was partially purified by column chromatography (SiO₂, petroleum ether/EtOAc=20:1 to 2:1) to give the desired compound (450 mg, crude) as an off-white solid that was used directly in the subsequent step.

LC-MS m/z 511.2 [M+H]⁺

¹H NMR (400 MHz, CDCl₃) δ 8.43 (d, J=2.3 Hz, 1H), 7.87-7.76 (m, 2H), 7.72-7.56 (m, 3H), 7.47 (d, J=8.2 Hz, 1H), 3.91-3.69 (m, 3H), 3.66-3.51 (m, 3H), 3.32-3.23 (m, 1H), 3.13-2.99 (m, 2H), 2.94-2.81 (m, 1H), 2.61-2.50 (m, 1H), 1.99-1.82 (m, 3H), 1.73-1.65 (m, 1H).

### 2-(((8R,9S,10S)-9-(6-bromopyridin-3-yl)-6-((4-nitrophenyl)sulfonyl)-1,6-diazabicyclo[6.2.0]decan-10-yl)methyl)isoindoline-1,3-dione (55.8)

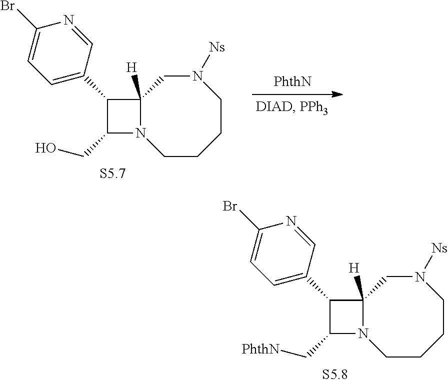

To a solution of S5.7 (450 mg, 880 μmol, 1.0 equiv) in THE (15 mL) was added triphenylphosphine (462 mg, 1.76 mmol, 2.0 equiv), isoindoline-1,3-dione (194 mg, 1.32 mmol, 1.5 equiv) and DIAD (356 mg, 1.76 mmol, 347 μL, 2.0 equiv) at 0° C. The mixture was stirred at 20° C. for 16 h. LC-MS showed the reaction was complete. The reaction mixture was poured into H₂O (25 mL) and extracted with EtOAc (15 mL×2). The combined organic layers were washed with brine (10 mL×2), dried over Na₂SO₄, filtered and concentrated under reduced pressure to give a residue. This residue was partially purified by column chromatography (SiO₂, petroleum ether/EtOAc=20:1 to 1:1) to give the desired compound (1.0 g) as a crude product that was used in the subsequent step without further purification.

LC-MS m/z 640.2 [M+H]⁺

### ((8R,9S,10S)-9-(6-bromopyridin-3-yl)-6-((4-nitrophenyl)sulfonyl)-1,6-diazabicyclo[6.2.0]decan-10-yl)methanamine (55.9)

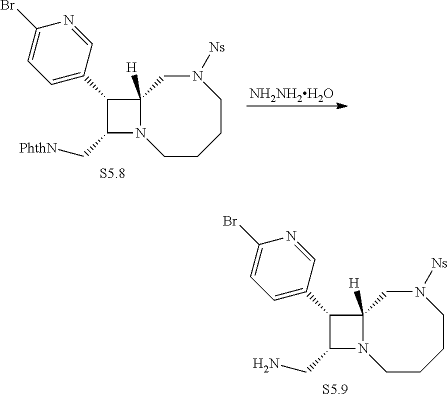

To a solution of S5.8 (1.0 g, crude) in EtOH (15 mL) was added N₂H₄.H₂O (117 mg, 2.34 mmol, 114 μL, 2.66 equiv). The mixture was stirred at 70° C. for 1 hour. LC-MS showed the reaction was complete. The reaction mixture was poured into H₂O (15 mL) and extracted with CH₂Cl₂ (15 mL×2). The combined organic layers were washed with brine (10 mL×2). dried over Na₂SO₄, filtered and concentrated under reduced pressure to give a residue This residue was purified by column chromatography (SiO₂, petroleum ether/EtOAc=2:1 then CH₂Cl₂/CH₃OH=100:1 to 20:1) to give the desired compound (400 mg, 784 μmol, 89% yield over two steps) as a yellow oil.

LC-MS m/z 510.2 [M+H]⁺

¹H NMR (400 MHz, CDCl₃) δ 8.45 (d, J=2.3 Hz, 1H), 7.85-7.74 (m, 2H), 7.71-7.60 (m, 2H), 7.59-7.55 (m, 1H), 7.48-7.45 (m, 1H), 3.90-3.79 (m, 1H), 3.73-3.61 (m, 2H), 3.38-3.29 (m, 1H), 3.28-3.21 (m, 1H), 3.14-2.96 (m, 3H), 2.86-2.73 (m, 2H), 2.69-2.59 (m, 1H), 2.51-2.39 (m, 1H), 1.88 (br s, 2H), 1.70-1.58 (m, 1H).

### 1-((8R,9S,10S)-9-(6-bromopyridin-3-yl)-6-((4-nitrophenyl)sulfonyl)-1,6-diaza bicyclo[6.2.0]decan-10-yl)-N,N-dimethylmethanamine (S5.10)

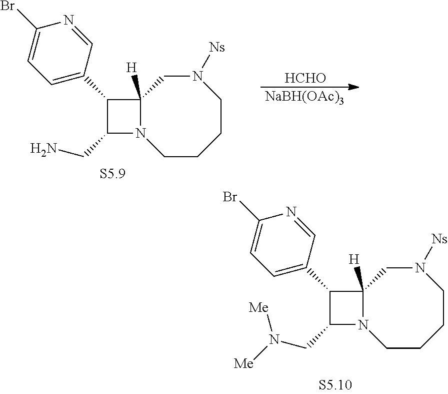

To a stirred solution of S5.9 (400 mg, 784 μmol, 1 equiv) in CH2Cl2 (15 mL) was added formaldehyde (37 wt. % in H2O, 636 mg, 7.84 mmol, 584 μL, 10 equiv) and MgSO4 (943 mg, 7.84 mmol, 10 equiv). The resulting reaction mixture was stirred at 25° C. for 0.5 h. To this mixture were added CH3COOH (47 mg, 784 μmol, 45 μL, 1.0 equiv) and NaBH(OAc)3 (831 mg, 3.92 mmol, 5 equiv). The resulting reaction mixture was stirred at 25° C. for 12.5 h. LC-MS showed the reaction was completed. The reaction mixture was quenched with H2O (20 mL) and extracted with CH2Cl2 (15 mL×3). The organic components were combined, dried over anhydrous Na2SO4 and concentrated to produce a residue. The residue was purified by preparative TLC (SiO2,CH2Cl2/CH3OH=10:1) to give the desired compound (330 mg, 613 μmol, 79% yield) as a yellow solid.

LC-MS m/z 538.2 [M+H]+

1H NMR (400 MHz, CDCl3) δ 8.36 (d, J=2.3 Hz, 1H), 7.72 (dd, J=1.7, 7.6 Hz, 1H), 7.64 (dd, J=2.5, 8.3 Hz, 1H), 7.57 (dquin, J=1.6, 7.3 Hz, 2H), 7.51-7.46 (m, 1H), 7.39 (d, J=8.2 Hz, 1H), 3.82-3.68 (m, 1H), 3.64-3.51 (m, 2H), 3.41 (dt, J=3.6, 7.3 Hz, 1H), 3.20-3.11 (m, 1H), 3.04-2.82 (m, 2H), 2.69 (dd, J=10.0, 14.6 Hz, 1H), 2.37-2.26 (m, 2H), 2.25-2.15 (m, 1H), 1.96 (s, 6H), 1.89-1.66 (m, 3H), 1.61-1.49 (m, 1H).

### N,N-dimethyl-1-((8R,9S,10S)-6-((4-nitrophenyl)sulfonyl)-9-(6-(phenylethynyl)pyridin-3-yl)-1,6-diazabicyclo[6.2.0]decan-10-yl)methanamine (S5.11)

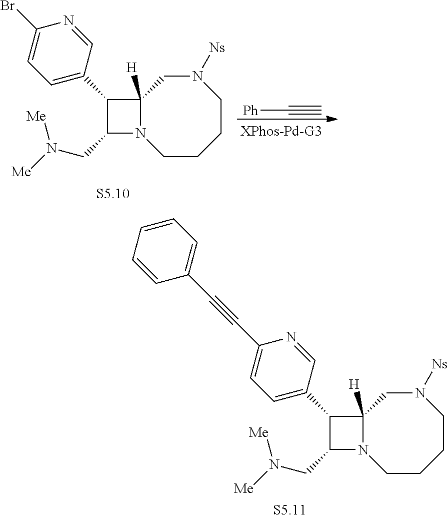

To a mixture of S5.10 (230 mg, 427 μmol, 1 equiv) and phenylacetylene (131 mg, 1.28 mmol, 141 μL, 3 equiv) in CH₃CN (5 mL) were added XPhos-Pd-G3 (36 mg, 43 μmol, 0.1 equiv) and Cs₂CO₃ (557 mg, 1.71 mmol, 4 equiv) in one portion at 70° C., and the mixture was stirred at this temperature for 2 hours. LC-MS showed the reaction was complete. The reaction mixture was quenched with H₂O (30 mL) and extracted with EtOAc (15 mL×3). The combined organic layers were washed with brine (20 mL×2), dried over Na₂SO₄, filtered, concentrated under reduced pressure to give a residue. The residue was purified by preparative TLC (SiO₂, CH₂Cl₂/CH₃OH=10:1) to give the desired compound (140 mg, 250 μmol, 59% yield) as a brown solid.

LC-MS m/z 560.4 [M+H]⁺

¹H NMR (400 MHz, CDCl₃) δ 8.71 (d, J=1.8 Hz, 1H), 7.84-7.75 (m, 2H), 7.65-7.59 (m, 4H), 7.58-7.54 (m, 1H), 7.51 (d, J=7.9 Hz, 1H), 7.40-7.36 (m, 3H), 3.90-3.80 (m, 1H), 3.74-3.63 (m, 2H), 3.52-3.45 (m, 1H), 3.24 (br d, J=13.6 Hz, 1H), 3.13-2.97 (m, 2H), 2.85-2.80 (m, 1H), 2.47-2.39 (m, 1H), 2.38-2.25 (m, 2H), 2.01 (s, 6H), 1.95-1.85 (m, 3H), 1.72-1.67 (m, 1H).

### N,N-dimethyl-1-((8R,9R,10S)-9-(6-(phenylethynyl)pyridin-3-yl)-1,6-diaza bicyclo[6.2.0]de-can-10-yl)methanamine (S5.12)

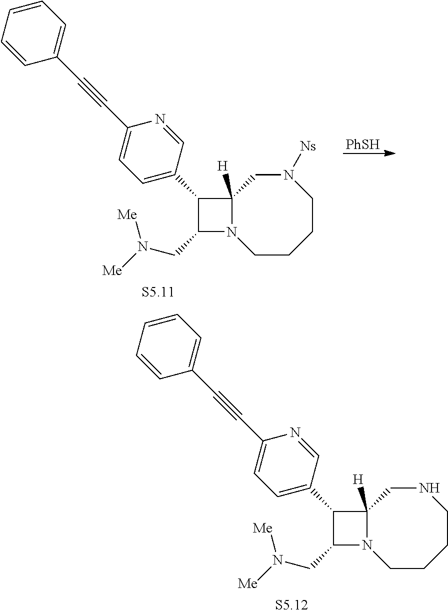

To a solution of S5.11 (140 mg, 250 μmol, 1.0 equiv) and benzenethiol (41 mg, 375 μmol, 38 μL, 1.50 equiv) in CH₃CN (5 mL) was added Cs₂CO₃ (98 mg, 300 μmol, 1.2 equiv) at 20° C. The mixture was stirred at 40° C. for 1h. LC-MS showed the reaction was complete. The reaction mixture was quenched with H₂O (10 mL) and then extracted with CH₂Cl₂ (5 mL×3). The combined organic layers were dried over Na₂SO₄, filtered and concentrated under reduced pressure to give a residue. This residue was purified by preparative TLC (SiO₂, CH₂Cl₂/CH₃OH=10:1) to give the desired compound (55 mg, 147 μmol, 59% yield) as a brown solid.

LC-MS m/z 375.4 [M+H]⁺

### (8R,9S,10S)-10-((dimethylamino)methyl)-N-(4-methoxyphenyl)-9-(6-(phenylethynyl)pyridin-3-yl)-1,6-diazabicyclo[6.2.0]decane-6-carboxamide (Compound 8)

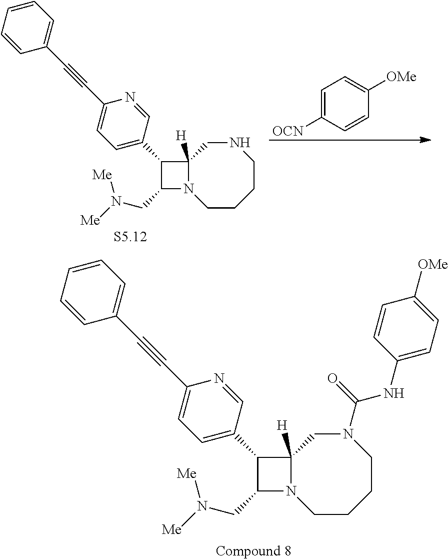

To a solution of S5.12 (55 mg, 147 μmol, 1.0 equiv) in CH₂Cl₂ (2 mL) were added triethylamine (15 mg, 147 μmol, 20 μL, 1.0 equiv) and 4-methoxyphenyl isocyanate (24 mg, 162 μmol, 21 μL, 1.1 equiv) at 0° C. The mixture was stirred at 25° C. for 1 h. LC-MS showed the reaction was complete. The reaction mixture was diluted with H₂O (10 mL), extracted with CH₂Cl₂ (5 mL×3), dried over Na₂SO₄, concentrated to give a residue. The residue was purified by preparative HPLC (buffer D) to afford Compound 8 (22 mg, 42 μmol, 29% yield) as a white solid.

LC-MS m/z 524.4 [M+H]⁺

¹H NMR (400 MHz, CDCl₃) δ 8.74 (s, 1H), 7.86 (dd, J=2.1, 8.0 Hz, 1H), 7.66-7.58 (m, 2H), 7.52 (d, J=8.0 Hz, 1H), 7.42-7.35 (m, 3H), 7.28 (s, 1H), 7.26-7.24 (m, 1H), 6.87-6.81 (m, 2H), 6.09 (s, 1H), 3.88-3.84 (m, 1H), 3.78 (s, 3H), 3.73-3.69 (m, 1H), 3.67-3.54 (m, 2H), 3.50-3.41 (m, 1H), 3.29-3.19 (m, 1H), 3.12-3.01 (m, 1H), 2.78 (dd, J=10.6, 14.2 Hz, 1H), 2.43-2.26 (m, 3H), 2.02 (s, 6H), 1.92-1.77 (m, 3H), 1.72-1.64 (m, 1H).

### Synthesis of Compound 4 ((3S,8R,9S,10S)-10-((dimethylamino)methyl)-3-hydroxy-N-(4-methoxyphenyl)-9-(4-(phenylethynyl)phenyl)-1,6-diazabicyclo[6.2.0]decane-6-carboxamide)

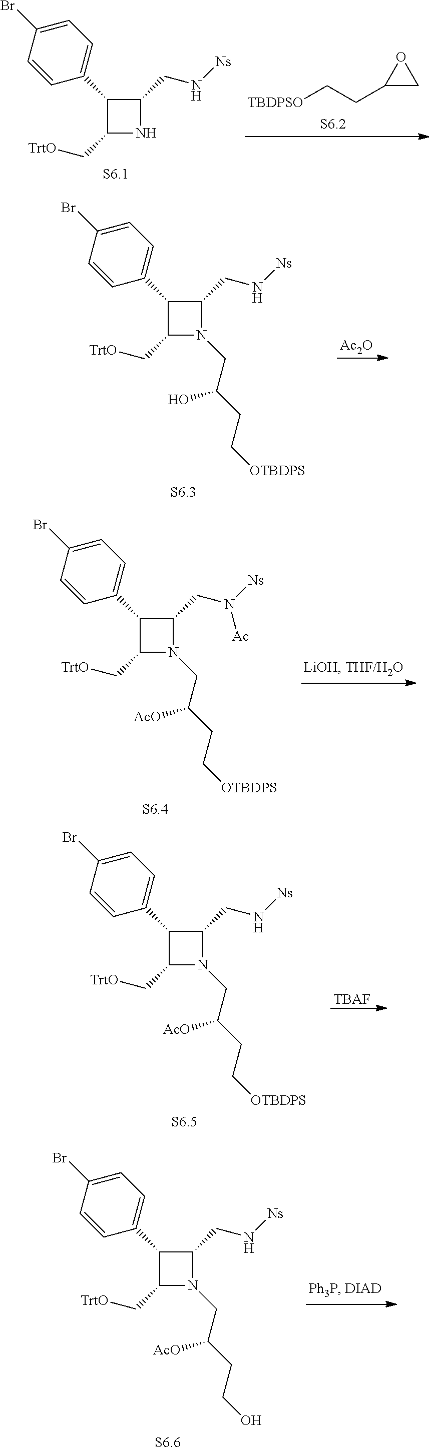

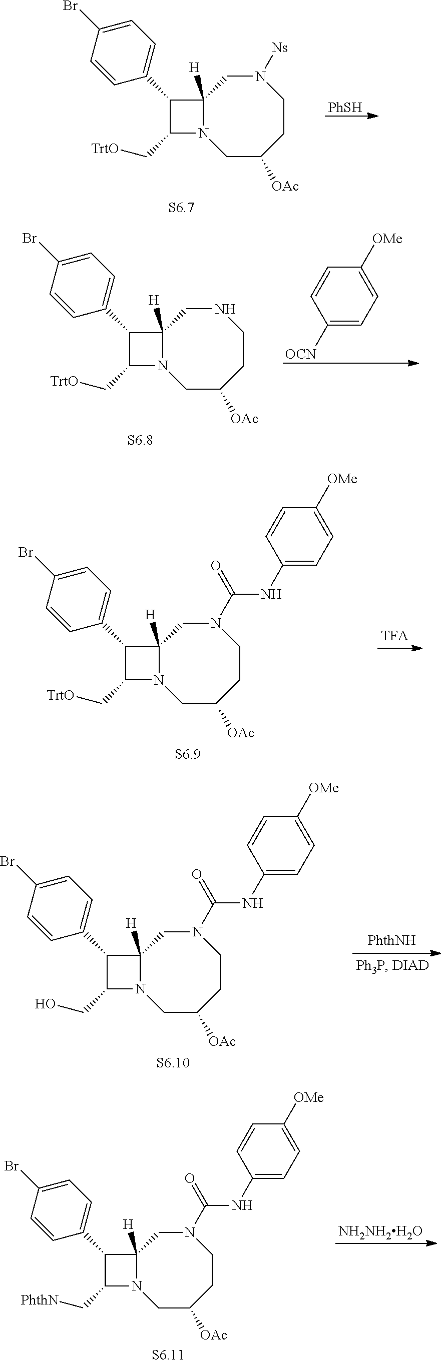

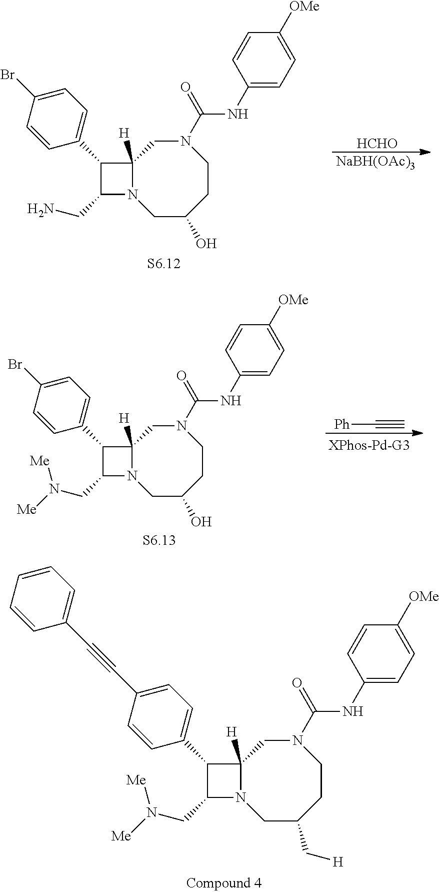

### N-(((2R,3R,4S)-3-(4-bromophenyl)-1-((S)-4-((tert-butyldiphenylsilyl)oxy)-2-hydroxybutyl)-4-((trityloxy)methyl)azetidin-2-yl)methyl)-4-nitrobenzenesulfonamide (S6.3)

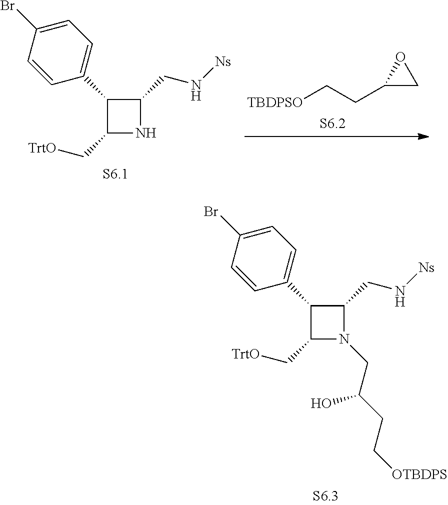

To a solution of S6.1 (400 mg, 573 μmol, 1.0 equiv) and LiClO₄ (121 mg, 50 μL, 1.15 mmol, 2.0 equiv) in CH₃CN (4 mL) was added S6.2 (374 mg, 1.15 mmol, 2.0 equiv). The mixture was stirred at 80° C. for 16 h. LC-MS showed the reaction was completed. The reaction mixture was concentrated under reduced pressure to remove solvent. The residue was diluted with H₂O (20 mL) and extracted with CH₂Cl₂ (10 mL×3). The combined organic layers were washed with brine (10 mL×3), dried over Na₂SO₄, filtered and concentrated under reduced pressure. The residue was purified by preparative TLC (petroleum ether/EtOAc=2:1) to give the desired compound (250 mg, 243.8 μmol, 43% yield) as a light yellow solid.

LC-MS m/z 1026.2 (base peak) [M+H]⁺

¹H NMR (400 MHz, CD₃OD) δ 7.85 (d, J=7.7 Hz, 1H), 7.81-7.75 (m, 1H), 7.74-7.60 (m, 6H), 7.51-7.38 (m, 6H), 7.23-7.05 (m, 19H), 5.84 (t, J=5.8 Hz, 1H), 3.86-3.70 (m, 3H), 3.68-3.55 (m, 3H), 3.19-3.24 (m, 1H), 3.05-3.1 (m, 1H), 2.95-2.85 (m, 2H), 2.65-2.55 (m, 2H), 1.74-1.60 (m, 1H), 1.54 (m, 1H), 1.07 (s, 9H).

### (S)-1-((2R,3R,4S)-3-(4-bromophenyl)-2-((N-((4-nitrophenyl)sulfonyl)acetamido)methyl)-4-((trityloxy)methyl)azetidin-1-yl)-4-((tert-butyldiphenylsilyl)oxy)butan-2-yl acetate (S6.4)

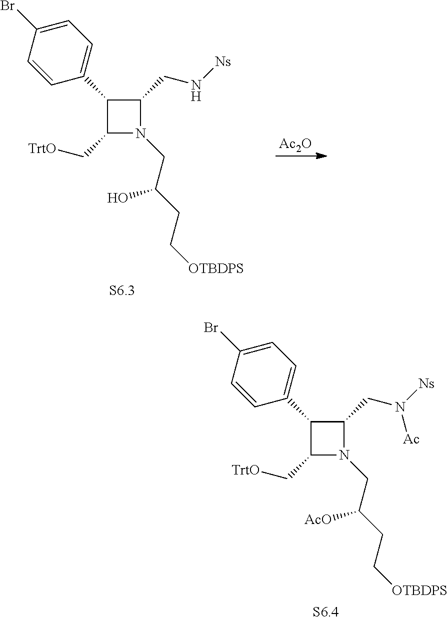

To a solution of S6.3 (250 mg, 244 μmol, 1.0 equiv), triethylamine (25 mg, 244 μmol, 34 μL, 1 equiv) and DMAP (3 mg, 24 μmol, 0.1 equiv) in CH₂Cl₂ (3.0 mL) was added acetic anhydride (62 mg, 609 μmol, 57 μL, 2.50 equiv). The mixture was stirred at 15° C. for 16 h upon which TLC analysis showed that the reaction was complete. The reaction mixture was quenched with H₂O (20 mL) and then extracted with CH₂Cl₂ (15 mL×3). The combined organic layers were washed with brine (10 mL), dried over Na₂SO₄, filtered and concentrated under reduced pressure to give a crude residue. The residue was purified by preparative TLC (petroleum ether/EtOAc=3:1) to give the desired product (270 mg, 243 μmol, 99.8% yield) as a white solid.

LC-MS m/z 1110.2 [M+H]⁺

### (S)-1-((2R,3R,4S)-3-(4-bromophenyl)-2-(((4-nitrophenyl)sulfonamido)methyl)-4-((trityloxy)methyl)azetidin-1-yl)-4-((tert-butyldiphenylsilyl)oxy)butan-2-yl acetate (S6.5)

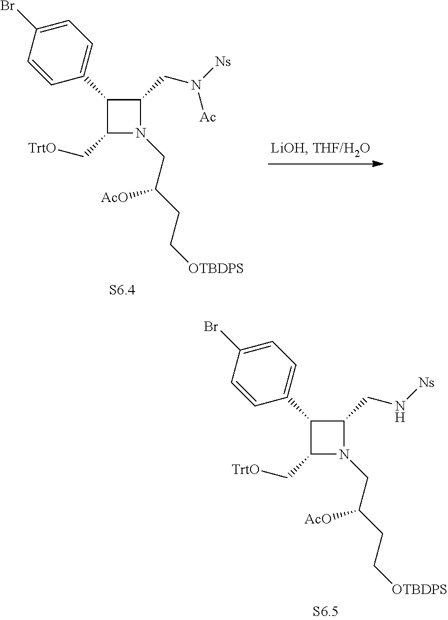

To a solution of S6.4 (270 mg, 243 μmol, 1.0 equiv) in THE (2.0 mL) and H₂O (2.0 mL) was added LiOH.H₂O (20 mg, 486 μmol, 2.0 equiv). The mixture was stirred at 15° C. for 1 h upon which LC-MS showed approx. 60% conversion of starting material. The reaction mixture was diluted with H₂O (30 mL) and extracted with CH₂Cl₂ (20 mL×4). The combined organic layers were washed with brine (20 mL×3), dried over Na₂SO₄, filtered and concentrated under reduced pressure to give a crude residue. This residue was purified by preparative TLC (petroleum ether/EtOAc=3:1) to give the desired compound (200 mg, 187 μmol, 77% yield) as a white solid.

LC-MS m/z 1068.3 [M+H]⁺

### (S)-1-((2R,3R,4S)-3-(4-bromophenyl)-2-(((4-nitrophenyl)sulfonamido)methyl)-4-((trityloxy)methyl)azetidin-1-yl)-4-hydroxybutan-2-yl acetate (S6.6)

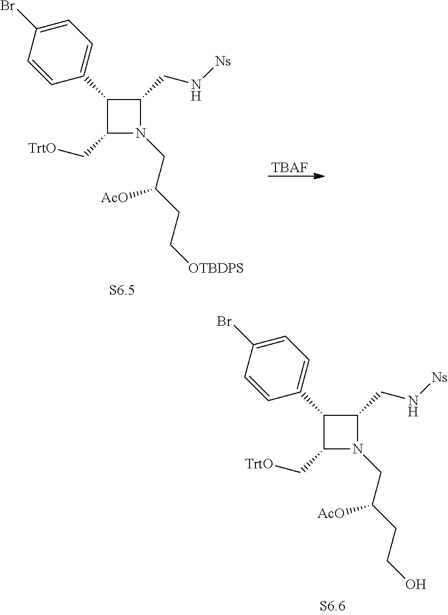

To a solution of S6.5 (200 mg, 187 μmol, 1.0 equiv) in THE (3.0 mL) was added TBAF (98 mg, 375 μmol, 2.0 equiv). The mixture was stirred at 15° C. for 2 h after which TLC analysis showed the reaction was complete. The reaction mixture was concentrated under reduced pressure. The residue was diluted with CH₂Cl₂ (30 mL), washed with H₂O (15 mL×5) and brine (10 mL×3), dried over Na₂SO₄, filtered and concentrated under reduced pressure to give a crude residue. This residue was purified by preparative TLC (petroleum ether/EtOAc=1:1) to give the desired compound (150 mg, 181 μmol, 97% yield) as a white solid.

LC-MS m/z 828.1 [M+H]⁺

### (3S,8R,9R,10S)-9-(4-bromophenyl)-6-((4-nitrophenyl)sulfonyl)-10-((trityloxy)methyl)-1,6-diazabicyclo[6.2.0]decan-3-yl acetate (S6.7)

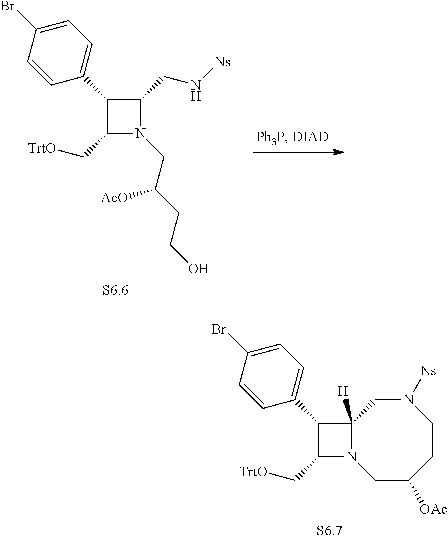

DIAD (73 mg, 362 μmol, 70 μL, 2.0 equiv) was added to a solution of triphenylphosphine (95 mg, 362 μmol, 2.0 equiv) in THE (2 mL) and this was stirred at 0° C. under N₂ atmosphere until a milky mixture was produced. This mixture was then added to a solution of S6.6 (150 mg, 181 μmol, 1.0 equiv) in THE (1 mL). The mixture was stirred at 15° C. for 16 h. LC-MS showed the reaction was complete. The reaction mixture was concentrated under reduced pressure. The resulting residue was diluted with CH₂Cl₂ (30 mL) washed with H₂O (10 mL×2), dried over Na₂SO₄, filtered and concentrated under reduced pressure to give a residue. This residue was partially purified by preparative TLC (petroleum ether/EtOAc=1:1) to give the desired compound (150 mg, crude) as a crude product that was used directly in the subsequent step.

LC-MS m/z 810.0 [M+H]⁺

### (3S,8R,9R,10S)-9-(4-bromophenyl)-10-((trityloxy)methyl)-1,6-diazabicyclo[6.2.0]decan-3-yl acetate (S6.8)

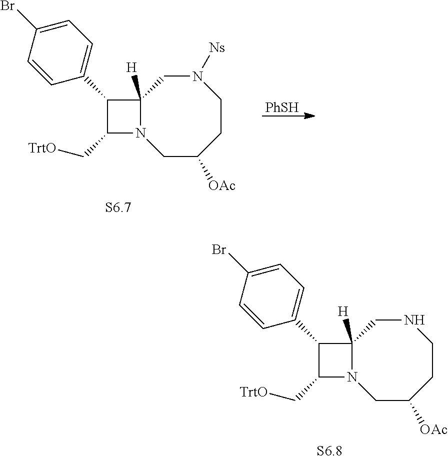

To a solution of S6.7 (150 mg, 185 μmol, 1.0 equiv) in CH3CN (3.0 mL) was added Cs₂CO₃ (121 mg, 370 μmol, 2.00 equiv) and benzenethiol (31 mg, 278 μmol, 28 μL, 1.50 equiv). The mixture was stirred at 40° C. for 2 h. LC-MS showed the reaction was complete. The reaction mixture was concentrated under reduced pressure to remove solvent. The residue was diluted with H₂O (30 mL) and extracted with CH₂Cl₂ (20 mL×5). The combined organic layers were washed with brine (20 mL×3), dried over Na₂SO₄, filtered and concentrated under reduced pressure to give a residue. This residue was partially purified by preparative TLC (petroleum ether/EtOAc=1:1) to give the desired compound (180 mg, crude) as a crude product that was used directly in the subsequent step.

### (3S,8R,9R,10S)-9-(4-bromophenyl)-6-((4-methoxyphenyl)carbamoyl)-10-((trityloxy)methyl)-1,6-diazabicyclo[6.2.0]decan-3-yl acetate (S6.9)

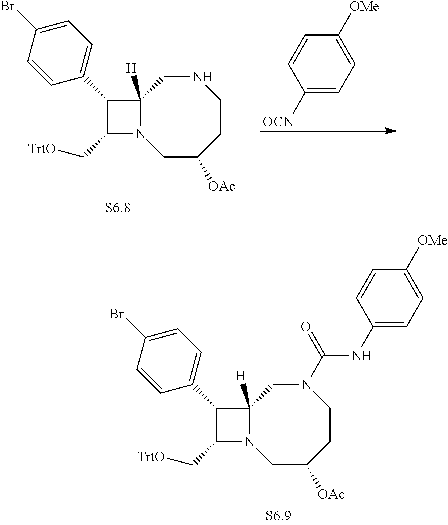

To a solution of S6.8 (180 mg, 288 μmol, 1.0 equiv) in CH₂Cl₂ (3.0 mL) was added 1-isocyanato-4-methoxybenzene (43 mg, 288 μmol, 36.99 μL, 1.00 equiv). The mixture was stirred at 15° C. for 1 h. LC-MS showed the reaction was complete. The reaction mixture was quenched with H₂O (15 mL) and then extracted with CH₂Cl₂ (20 mL×4). The combined organic layers were washed with saturated NaHCO₃ (15 mL), brine (15 mL×2), dried over Na₂SO₄, filtered and concentrated under reduced pressure to give a residue. This residue was purified by preparative TLC (petroleum ether/EtOAc=1:1) to give the desired compound (200 mg, 258 μmol, 90% yield) as a white solid.

LC-MS m/z 774.2 [M+H]⁺

¹H NMR (400 MHz, CDCl₃) δ=7.31-7.14 (m, 21H), 6.82-6.78 (m, 2H), 4.75-4.71 (m, 1H), 3.76-3.73 (m, 4H), 3.69-3.45 (m, 4H), 3.33-3.23 (m, 1H), 3.16-3.07 (m, 2H), 2.97-2.89 (m, 1H), 2.75-2.65 (m, 1H), 2.58 (dd, J=4.0, 12.6 Hz, 1H), 2.31-2.23 (m, 1H), 1.93 (s, 3H), 1.86-1.83 (m, 1H).

### (3S,8R,9R,10S)-9-(4-bromophenyl)-10-(hydroxymethyl)-6-((4-methoxyphenyl)carbamoyl)-1,6-diazabicyclo[6.2.0]decan-3-yl acetate (S6.10)

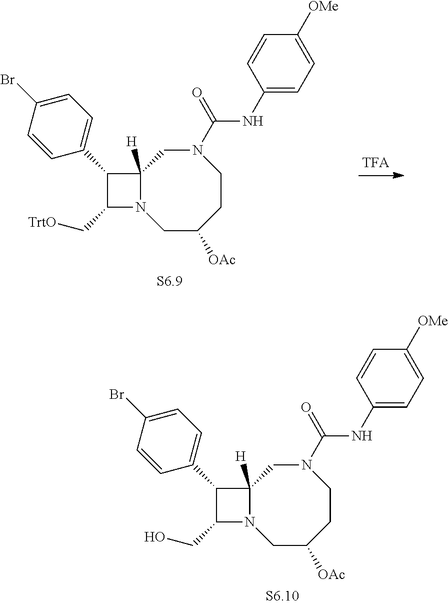

To a solution of S6.9 (200 mg, 258 μmol, 1.00 equiv) in CH₂Cl₂ (3.0 mL) was added TFA (294.34 mg, 2.58 mmol, 197 μL, 10.00 equiv,). The mixture was stirred at 15° C. for 2 h. LC-MS showed the reaction was complete. The reaction mixture was quenched with saturated NaHCO₃ solution (20 mL) and extracted with CH₂Cl₂ (20 mL×5). The combined organic layers were washed with brine (10 mL×3), dried over Na₂SO₄, filtered and concentrated under reduced pressure to give a residue. The residue was purified by column chromatography on silica gel (petroleum ether/EtOAc=1:1) to give the desired compound (130 mg, 244 μmol, 95% yield) as a white solid.

LC-MS m/z 532.3 [M+H]⁺

¹H NMR (400 MHz, CDCl₃) δ 7.42-7.36 (m, 2H), 7.34-7.28 (m, 2H), 7.22-7.19 (m, 2H), 6.80-6.73 (m, 2H), 6.24 (s, 1H), 4.76-4.2 (m, 1H), 3.83-3.67 (m, 5H), 3.65-3.30 (m, 7H), 3.18 (dd, J=7.2, 13.2 Hz, 1H), 2.85-2.75 (m, 1H), 2.65 (dd, J=2.9, 13.4 Hz, 1H), 2.28-2.14 (m, 1H), 2.09 (s, 3H), 1.95-1.84 (m, 1H).

### (3S,8R,9S,10S)-9-(4-bromophenyl)-10-((1,3-dioxoisoindolin-2-yl)methyl)-6-((4-methoxyphenyl)carbamoyl)-1,6-diazabicyclo[6.2.0]decan-3-yl acetate (S6.11)

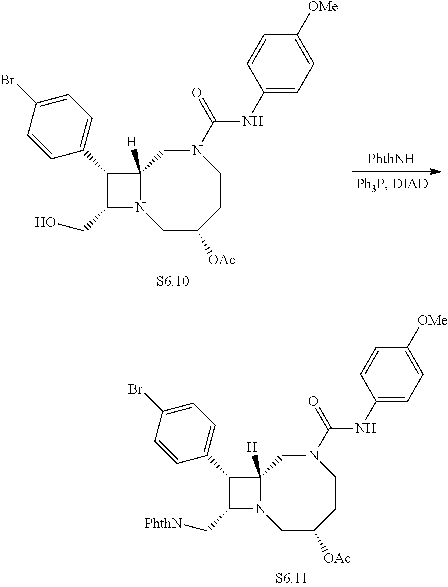

A mixture of triphenylphosphine (128 mg, 488 μmol, 2.00 equiv) and DIAD (99 mg, 488 μmol, 95 μL, 2.00 eq) in THE (1 mL) was stirred at 0° C. under N₂ atmosphere until a milky mixture was observed. Then, this mixture was added to the solution of S6.10 (130 mg, 244 μmol, 1.0 equiv) and N-methylphthalimide (54 mg, 366 μmol, 1.50 equiv) in THF (1 mL) at 0° C. The mixture was stirred at 15° C. for 12 h. LC-MS showed the reaction was complete. The reaction mixture was concentrated under reduced pressure. This residue was partially purified by preparative TLC (petroleum ether/EtOAc=1:1) to give the desired compound (200 mg, crude) as a crude product that was used directly in the subsequent step.

### (3S,8R,9S,10S)-10-(aminomethyl)-9-(4-bromophenyl)-3-hydroxy-N-(4-methoxyphenyl)-1,6-diazabicyclo[6.2.0]decane-6-carboxamide (S6.12)

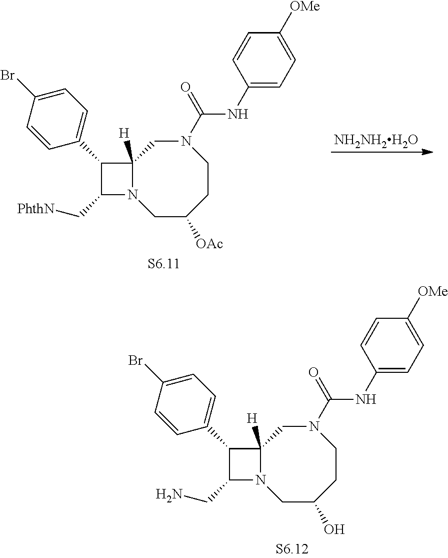

To a solution of S6.11 (200 mg, 302 μmol, 1.0 equiv) in EtOH (2.0 mL) was added N₂H₄.H₂O (97% w/w, 30 mg, 0.60 mmol, 29 μL, 2.0 equiv). The mixture was stirred at 70° C. for 2 h. LC-MS showed the reaction was complete. The reaction mixture was concentrated under reduced pressure to remove solvent. The residue was diluted with H₂O (10 mL) and extracted with CH₂Cl₂ (20 mL×5). The combined organic layers were washed with brine (10 mL×2), dried over Na₂SO₄, filtered and concentrated under reduced pressure to give a residue. The residue was purified by column chromatography on silica gel (CH₂Cl₂/CH₃OH=10:1) to give the desired compound (60 mg, 123 μmol, 41% yield) as a white solid.

LC-MS m/z 489.2 [M+H]⁺

¹H NMR (400 MHz, CDCl₃) δ 7.41 (d, J=8.4 Hz, 2H), 7.26 (d, J=8.5 Hz, 2H), 7.22-7.18 (s, 2H), 6.82-6.71 (m, 2H), 6.06 (s, 1H), 3.82-3.75 (m, 1H), 3.71 (s, 3H), 3.69-3.54 (m, 4H), 3.53-3.35 (m, 2H), 3.06 (dd, J=6.7, 13.7 Hz, 1H), 2.85-2.65 (m, 4H), 2.02-1.77 (m, 2H)

### (3S,8R,9S,10S)-9-(4-bromophenyl)-10-((dimethylamino)methyl)-3-hydroxy-N-(4-methoxyphenyl)-1,6-diazabicyclo[6.2.0]decane-6-carboxamide (S6.13)

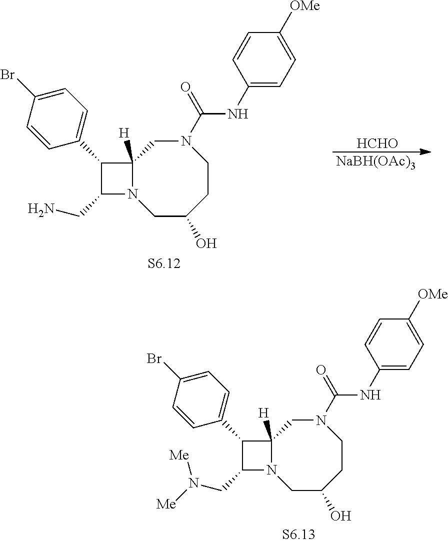 [SMILES: COc1ccc(NC(=O)N2CC[C@H](O)CN3[C@H](CN)[C@H](c4ccc(Br)cc4)[C@@H]3C2)cc1 ; "HCHO" ; COc1ccc(NC(=O)N2CC[C@H](O)CN3[C@H](CN(C)C)[C@H](c4ccc(Br)cc4)[C@@H]3C2)cc1 ; "NaBH(OAc)3"]

To a mixture of S6.12 (60 mg, 123 μmol, 1.0 equiv), formalin (formaldehyde 37% w/w in H₂O, 60 mg, 0.74 mmol, 55 μL, 6.0 equiv), MgSO₄ (148 mg, 1.23 mmol, 10.0 equiv), and AcOH (1 mg, 0.01 μmol, 0.7 μL, 0.1 equiv) in CH₂Cl₂ (3.0 mL) was added NaBH(OAc)₃ (78 mg, 368 μmol, 3.0 equiv). The mixture was stirred at 15° C. for 2 h. LC-MS showed the reaction was complete. The reaction mixture was filtered and washed with NaHCO₃ solution (10 mL×3). The combined organic layers were dried over Na₂SO₄, filtered and concentrated under reduced pressure to give the desired compound (50 mg, 97 μmol, 79% yield) as a white solid.

LC-MS m/z 517.3 [M+H]⁺

¹H NMR (400 MHz, CDCl₃) δ 7.46 (d, J=8.4 Hz, 2H), 7.30-7.23 (m, 4H), 6.86-6.77 (m, 2H), 6.12 (s, 1H), 3.89-3.80 (m, 1H), 3.79-3.73 (m, 3H), 3.73-3.57 (m, 5H), 3.54-3.40 (m, 1H), 3.14-3.1 (m, 1H), 2.83-2.74 (m, 1H), 2.70-2.64 (m, 1H), 2.46-2.26 (m, 2H), 2.04 (s, 6H), 2.00-1.86 (m, 2H).

### (3S,8R,9S,10S)-10-((dimethylamino)methyl)-3-hydroxy-N-(4-methoxyphenyl)-9-(4-(phenylethynyl)phenyl)-1,6-diazabicyclo[6.2.0]decane-6-carboxamide (Compound 4)

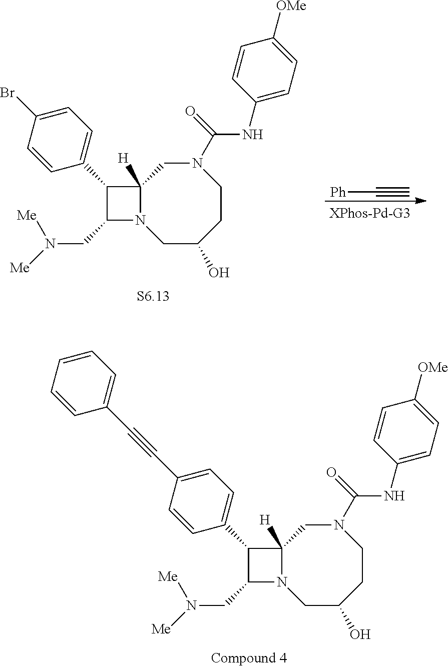

To a solution of S6.13 (50 mg, 97 μmol, 1.0 equiv) and phenylacetylene (30 mg, 290 μmol, 32 μL, 3.0 equiv) in CH₃CN (1.0 mL) was added Cs₂CO₃ (126 mg, 387 μmol, 4.0 equiv) and XPhos-Pd-G3 (8.2 mg, 9.7 μmol, 0.10 equiv). The mixture was stirred under a N2 atmosphere at 70° C. for 1 h. LC-MS showed that the reaction was complete. The reaction mixture was concentrated under reduced pressure to remove solvent. The reaction mixture was purified by preparative TLC (CH₂Cl₂/CH₃OH=10:1) followed by preparative HPLC (buffer C) to give the desired product (10.0 mg, 17 μmol, 19% yield) as a white solid.

LC-MS m/z 539.4 [M+H]⁺

¹H NMR (400 MHz, CDCl₃) δ 8.44 (br s, 1H), 7.54 (br d, J=7.0 Hz, 4H), 7.44-7.34 (m, 4H), 7.29-7.24 (m, 2H), 6.83 (br d, J=8.8 Hz, 2H), 6.29 (s, 1H), 5.71 (br s, 1H), 3.87 (br s, 2H), 3.77 (s, 3H), 3.75-3.62 (m, 4H), 3.52-3.38 (m, 1H), 3.20-3.16 (m, 1H), 2.93-2.79 (m, 1H), 2.77-2.66 (m, 2H), 2.66-2.58 (m, 1H), 2.19 (s, 6H), 2.08-1.97 (m, 1H), 1.96-1.85 (m, 1H).

### Synthesis of Compound 18

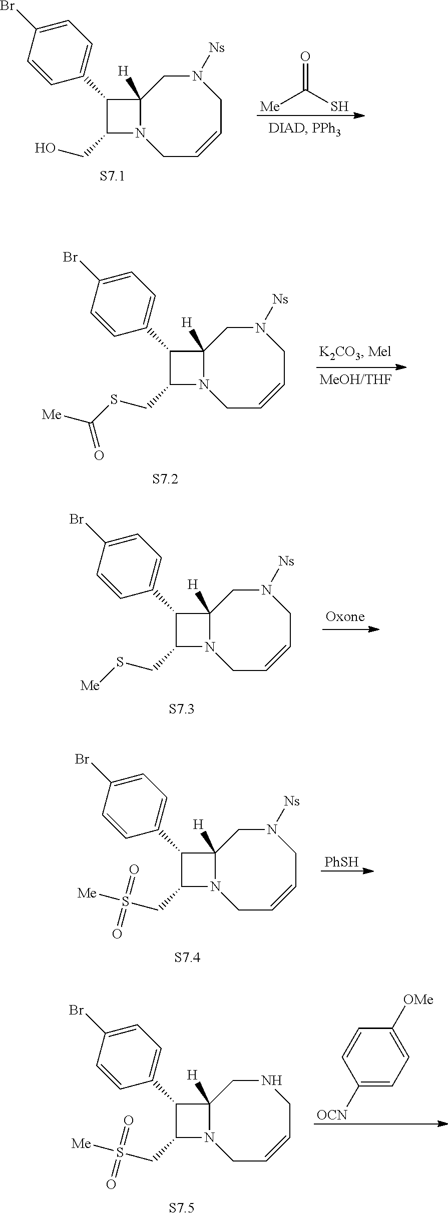

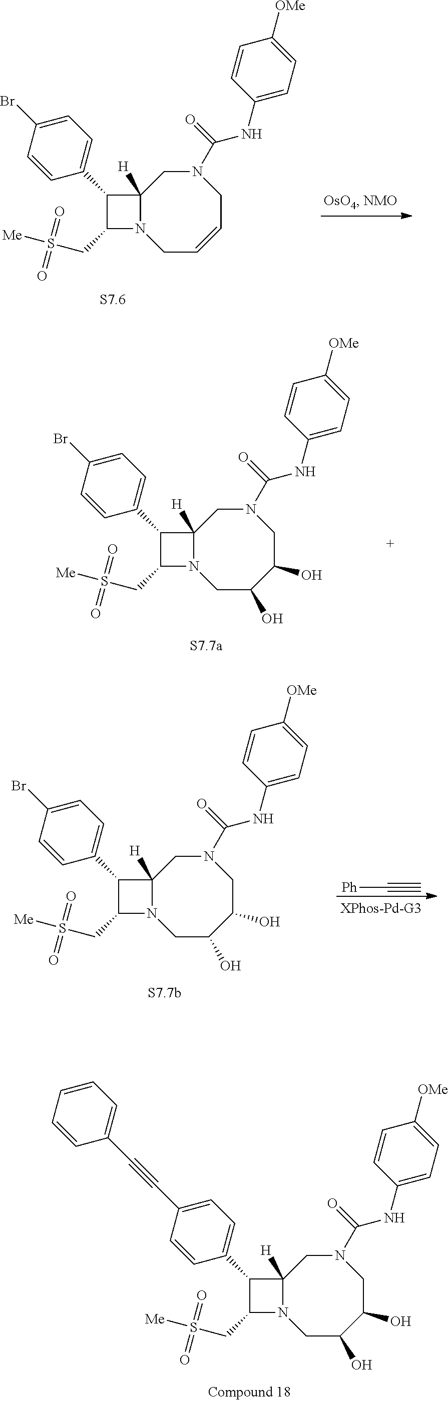

### S-(((8R,9R,10S,Z)-9-(4-bromophenyl)-6-((4-nitrophenyl)sulfonyl)-1,6-diazabicyclo[6.2.0]dec-3-en-10-yl)methyl) ethanethioate (S7.2)

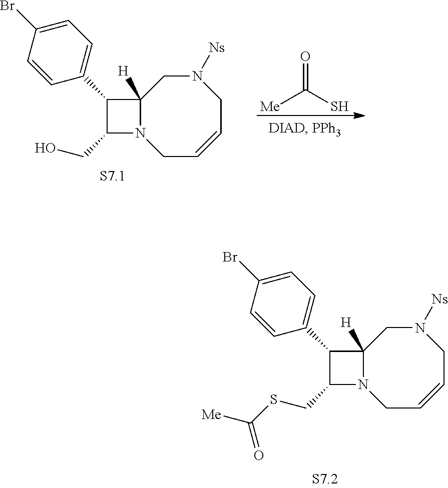

A stirred solution of triphenylphosphine (1.29 g, 4.92 mmol, 2.2 equiv) in THE (37 mL) was cooled to 0° C. and to this was added diisopropyl azodicarboxylate (DIAD) (970 μL, 4.92 mmol, 2.2 equiv) under nitrogen. The mixture was stirred at this temperature for 10 minutes and a solution of S7.1 (1.14 g, 2.24 mmol, 1 equiv)⁴ in THE (8 mL) was added dropwise. The mixture was stirred at 0° C. for 10 minutes and then a solution of thioacetic acid (350 μL, 4.92 mmol, 2.2 equiv) in THE (3 mL) was added. The mixture was warmed to room temperature, stirred for 1 hour and was concentrated. The residue was purified by column chromatography on silica gel (petroleum ether/EtOAc=0:1 to 1:1) to give S7.2 (1.325 g) as a yellow oil. NMR analysis revealed contamination with diisopropyl 1,2-hydrazinedicarboxylate (S7.2′)⁵—estimated w/w purity: 64%; calculated yield of S7.2: 67%.

LC-MS m/z 566.1 [M+H]⁺

¹H NMR (400 MHz, CDCl₃) δ 7.89 (d, J=7.5 Hz, 1H), 7.72-7.55 (m, 3H), 7.44 (d, J=8.0 Hz, 2H), 7.30 (d, J=8.1 Hz, 2H), 6.32 (br s, 2H, S7.2′), 5.98-5.85 (m, 1H), 5.77-5.66 (m, 1H), 4.98 (hept, J=6.3 Hz, 2H, S7.2′), 4.20-4.03 (m, 1H), 3.96 (dd, J=14.7, 9.5 Hz, 1H), 3.54 (dq, J=17.0, 7.6 Hz, 3H), 3.37 (q, J=7.2 Hz, 1H), 3.30 (d, J=14.3 Hz, 1H), 3.21-3.00 (m, 3H), 2.60 (dd, J=13.7, 8.5 Hz, 1H), 2.25 (s, 3H), 1.26 (d, J=6.4 Hz, 12H, S7.2′).

### (8R,9R,10S,Z)-9-(4-bromophenyl)-10-((methylthio)methyl)-6-((4-nitrophenyl)sulfonyl)-1,6-diazabicyclo[6.2.0]dec-3-ene (S7.3)

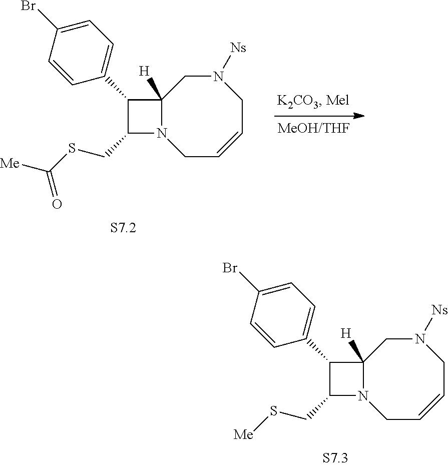

To a solution of S7.2 (841 mg, 1.49 mmol, 1 equiv) in THF/CH₃OH (1:1, 18 mL) was added K₂CO₃ (411 mg, 2.98 mmol, 2 equiv) and Mel (460 μL, 7.45 mmol, 5 equiv). The mixture was stirred at rt for 1 h, then H₂O was added (15 mL) and the mixture was extracted with EtOAc (20 mL×3). The combined organic layers were washed with brine, dried over Na₂SO₄ and concentrated under reduced pressure. The residue was purified by column chromatography (hexane/EtOAc=0:1 to 1:1) to give S7.3 (1.03 g) as a yellow oil. NMR analysis revealed contamination with diisopropyl 1,2-hydrazinedicarboxylate (S7.2′)⁵—estimated w/w purity: 58%; calculated yield of S7.3: 74%.

LC-MS m/z 538.2 [M+H]⁺

¹H NMR (400 MHz, CDCl₃) δ 7.89 (dd, J=7.5, 1.8 Hz, 1H), 7.72-7.55 (m, 3H), 7.43 (d, J=8.4 Hz, 2H), 7.31 (d, J=8.4 Hz, 2H), 6.31 (br s, 4H, S7.2′), 5.95-5.86 (m, 1H), 5.78-5.66 (m, 1H), 4.98 (hept, J=6.3 Hz, 2H, S7.2′), 4.10 (dd, J=15.6, 6.8 Hz, 1H), 3.97 (dd, J=14.6, 9.4 Hz, 1H), 3.64-3.49 (m, 3H), 3.45 (q, J=7.3 Hz, 1H), 3.29 (d, J=14.4 Hz, 1H), 3.19-3.06 (m, 2H), 2.53 (dd, J=13.4, 6.0 Hz, 1H), 2.36 (dd, J=13.4, 7.8 Hz, 1H), 1.90 (s, 3H), 1.27 (d, J=6.3 Hz, 12H, S7.2′).

### (8R,9R,10S,Z)-9-(4-bromophenyl)-10-((methylsulfonyl)methyl)-6-((4-nitrophenyl)sulfonyl)-1,6-diazabicyclo[6.2.0]dec-3-ene (S7.4)

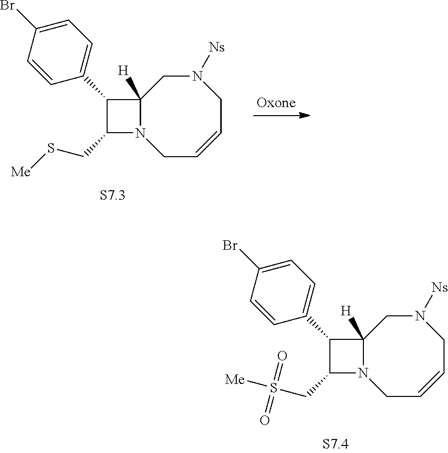

To a precooled (0° C.) solution of S7.3 (597 mg, 1.1 mmol, 1 equiv) in CH₃OH (11 mL) and THE (11 mL) was added oxone (1.5 g, 4.40 mmol, 4 equiv). The mixture was stirred at this temperature for 1 h then cold sat. aq. Na₂SO₃ (20 mL) was added. The mixture was extracted EtOAc (3×40 mL). The combined organic layers were washed with brine, dried over Na₂SO₄, filtered and concentrated under reduced pressure. The residue was purified by column chromatography (hexane/EtOAc=100:0 to 25:75) to afford S7.4 (329 mg, 52%) as a colorless oil.

LC-MS m/z 570.11 [M+H]⁺

¹H NMR (300 MHz, CDCl₃) δ 7.94-7.89 (m, 1H), 7.73-7.57 (m, 3H), 7.48 (d, J=8.4 Hz, 2H), 7.32 (d, J=8.2 Hz, 2H), 5.93 (dt, J=12.1, 6.3 Hz, 1H), 5.72 (q, J=9.3 Hz, 1H), 4.16-3.87 (m, 3H), 3.81-3.50 (m, 3H), 3.36 (d, J=14.4 Hz, 1H), 3.25-2.93 (m, 4H), 2.69 (s, 3H).

### (8R,9R,10S,Z)-9-(4-bromophenyl)-10-((methylsulfonyl)methyl)-1,6-diazabicyclo[6.2.0]dec-3-ene (S7.5)

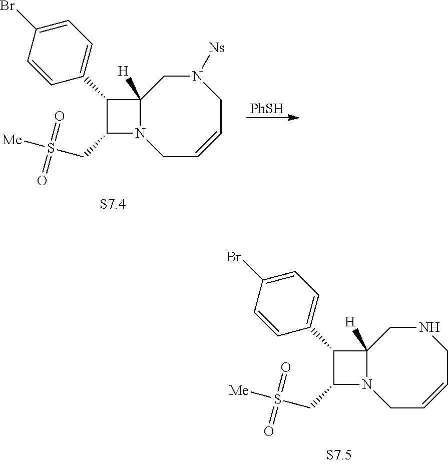

To a solution of S7.4 (364 mg, 638 μmol, 1.00 equiv) and Cs₂CO₃ (413 mg, 1.27 mmol, 1.99 equiv) in CH₃CN (15 mL) was added thiophenol (98 μL, 0.96 mmol, 1.5 equiv). The mixture was heated to 40° C. and stirred for 2 h, then it was allowed to cool to rt and H₂O (15 mL) was added. The mixture was extracted EtOAc (3×20 mL). The combined organic layers were washed with brine, dried over Na₂SO₄, filtered and concentrated under reduced pressure. The residue was purified by column chromatography (hexane/EtOAc=100:0 to 0:100 then CH₂Cl₂/CH₃OH 100:0 to 80:20) to afford S7.5 (213 mg, 86%) as a yellow oil.

LC-MS m/z 385.13 [M+H]⁺

¹H NMR (400 MHz, CDCl₃) δ 7.45 (d, J=8.3 Hz, 2H), 7.33 (d, J=8.2 Hz, 2H), 5.98 (dt, J=10.9, 6.9 Hz, 1H), 5.71 (td, J=10.4, 6.5 Hz, 1H), 4.08-3.91 (m, 1H), 3.80-3.68 (m, 1H), 3.69-3.58 (m, 2H), 3.48 (dd, J=13.6, 7.3 Hz, 1H), 3.32 (dd, J=13.9, 6.7 Hz, 1H), 3.21-3.08 (m, 1H), 3.08-2.85 (m, 3H), 2.69 (s, 3H), 2.55 (d, J=13.6 Hz, 1H).

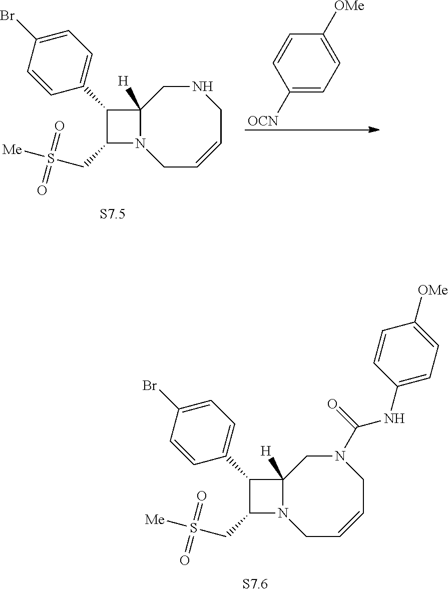

### (8R,9R,10S,Z)-9-(4-bromophenyl)-N-(4-methoxyphenyl)-10-((methylsulfonyl)methyl)-1,6-diazabicyclo[6.2.0]dec-3-ene-6-carboxamide (S7.6)

To a precooled (0° C.) solution of S7.5 (213 mg, 550 μmol, 1.00 equiv) in CH₂Cl₂ (27.4 mL) were added triethylamine (151 μL, 1.09 mmol, 1.98 equiv) then 4-methoxyphenyl isocyanate (85 μL, 659 μmol, 1.2 equiv). The mixture was stirred at 0° C. for 90 min then concentrated under reduced pressure. The residue was purified by column chromatography (hexane/EtOAc=100:0 to 30:70) to afford S7.6 (268 mg, 92%).

LC-MS m/z 533.76 [M+H]⁺

¹H NMR (300 MHz, CDCl₃) δ 7.57-7.46 (m, 2H), 7.37-7.29 (m, 2H), 7.24-7.16 (m, 2H), 6.86-6.75 (m, 2H), 5.91-5.69 (m, 2H), 4.19-3.93 (m, 3H), 3.82-3.61 (m, 7H), 3.27-3.18 (m, 1H), 3.13 (d, J=5.9 Hz, 2H), 2.84 (dd, J=13.8, 10.1 Hz, 1H), 2.67 (s, 3H).

### (3S,4R,8R,9R,10S)-9-(4-bromophenyl)-3,4-dihydroxy-N-(4-methoxyphenyl)-10-((methylsulfonyl)methyl)-1,6-diazabicyclo[6.2.0]decane-6-carboxamide (S7.7a)

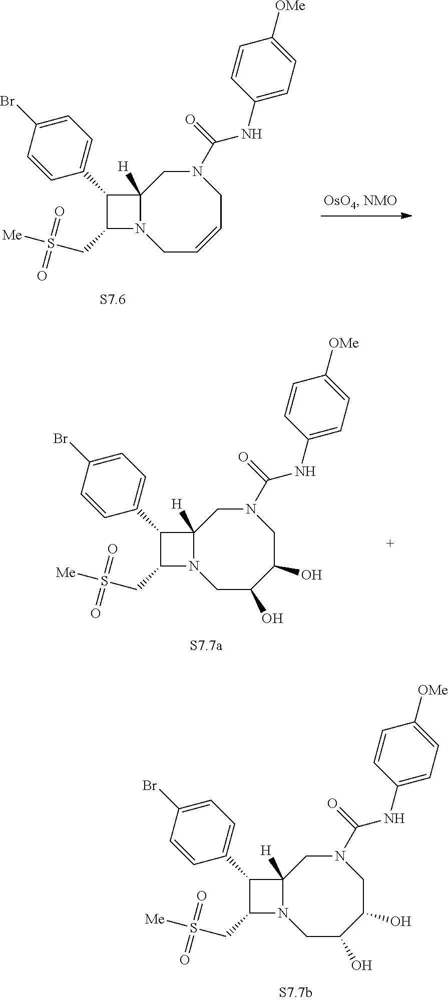 [SMILES: COc1ccc(NC(=O)N2C/C=C\CN3[C@H](CS(C)(=O)=O)[C@H](c4ccc(Br)cc4)[C@@H]3C2)cc1 ; COc1ccc(NC(=O)N2C[C@@H](O)[C@@H](O)CN3[C@H](CS(C)(=O)=O)[C@H](c4ccc(Br)cc4)[C@@H]3C2)cc1 ; COc1ccc(NC(=O)N2C[C@H](O)[C@H](O)CN3[C@H](CS(C)(=O)=O)[C@H](c4ccc(Br)cc4)[C@@H]3C2)cc1]

To a solution of S7.6 (268 mg, 501 μmol, 1.00 equiv) in a mixture of acetone (4.2 mL) and H₂O (0.81 mL) were added NMO (50% w/w in H₂O, 0.21 mL, 1.0 mmol, 2.0 equiv) and OsO₄ (4% w/w in H₂O, 30 μL, 5.0 μmol, 0.010 equiv). The mixture was stirred at rt for 16 h then dried over Na₂SO₄ and concentrated under reduced pressure. The crude product was purified by column chromatography (hexane/EtOAc=100:0 to 0:100) to afford S7.7a (85 mg, 30%) and S7.7b (139 mg, 48%).

S7.7a

LC-MS m/z 568.20 [M+H]⁺

¹H NMR (400 MHz, CDCl₃) δ 7.50 (d, J=8.3 Hz, 2H), 7.34 (d, J=8.1 Hz, 2H), 7.23-7.18 (m, 2H), 6.86-6.78 (m, 2H), 4.31 (dd, J=15.9, 5.4 Hz, 1H), 4.19 (q, J=6.4 Hz, 1H), 4.09 (br s, 1H), 4.06-3.94 (m, 1H), 3.93-3.83 (m, 1H), 3.77 (s, 3H), 3.69-3.52 (m, 2H), 3.29-3.12 (m, 2H), 3.08-2.84 (m, 3H), 2.75 (s, 3H), 2.70-2.58 (m, 1H), 2.13 (br s, 1H).

S7.7b

LC-MS m/z 568.16 [M+H]⁺

¹H NMR (400 MHz, CDCl₃) δ 7.52 (d, J=8.4 Hz, 2H), 7.28 (d, J=8.5 Hz, 2H), 7.23 (d, J=8.9 Hz, 2H), 6.82 (d, J=9.0 Hz, 2H), 4.21 (q, J=7.1 Hz, 1H), 3.92-3.83 (m, 1H), 3.76 (s, 3H), 3.75-3.63 (m, 3H), 3.61-3.33 (m, 4H), 3.18 (dd, J=14.2, 5.7 Hz, 1H), 3.12-2.99 (m, 2H), 2.94-2.81 (m, 1H), 2.76 (d, J=14.5 Hz, 1H), 2.72 (s, 3H).

### (3S,4R,8R,9R,10S)-3,4-dihydroxy-N-(4-methoxyphenyl)-10-((methylsulfonyl)methyl)-9-(4-(phenylethynyl)phenyl)-1,6-diazabicyclo[6.2.0]decane-6-carboxamide (Compound 18)

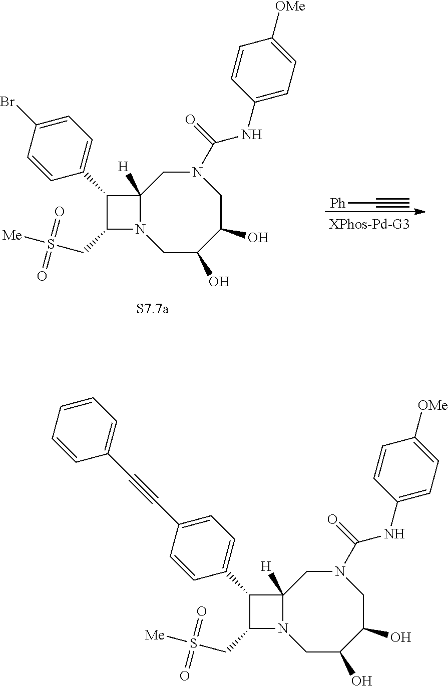

A sealed vial containing S7.7a (39 mg, 68 μmol, 1.0 equiv) was evacuated and backfilled with N₂ (×3) then were added CH₃CN (0.69 mL) previously sparged with argon for 40 min, triethylamine (38 μL, 0.27 mmol, 4.0 equiv) and phenylacetylene (37 μL, 0.34 mmol, 5.0 equiv), followed by XPhos-Pd-G3 (5.8 mg, 6.8 μmol, 0.1 equiv). The vial was sealed, heated to 70° C. and stirred for 90 min. The reaction was allowed to cool to rt then sat. aq. NaHCO₃ (1 mL) was added and the mixture was extracted with CH₂Cl₂ (2 mL). The combined organic layers were dried over Na₂SO₄, filtered and concentrated under reduced pressure. The residue was purified by reverse phase chromatography (H₂O/CH₃CN=100:0 to 50:50, buffered with 0.1% TFA) to afford Compound 18 (20 mg, 50%).

LC-MS m/z 590.68 [M+H]⁺

¹H NMR (400 MHz, CDCl₃) δ 7.59-7.50 (m, 4H), 7.44 (d, J=8.3 Hz, 2H), 7.40-7.33 (m, 3H), 7.24-7.18 (m, 2H), 6.87-6.78 (m, 2H), 4.31 (dd, J=16.1, 5.4 Hz, 1H), 4.20 (q, J=6.5 Hz, 1H), 4.14-4.07 (m, 1H), 4.02 (br d, J=14.3 Hz, 1H), 3.94-3.86 (m, 1H), 3.77 (s, 3H), 3.68 (t, J=7.5 Hz, 1H), 3.65-3.57 (m, 1H), 3.52 (br s, 1H), 3.31-3.16 (m, 2H), 3.07-2.95 (m, 2H), 2.91 (dd, J=13.8, 2.8 Hz, 1H), 2.72 (s, 3H), 2.70-2.63 (m, 1H), 2.27-2.20 (m, 1H), 1 exchangeable proton not observed.

### Synthesis of Compound 5 ((8R,9R,10S)-10-((dimethylamino)methyl)-10-(hydroxymethyl)-N-(4-methoxyphenyl)-9-(4-(phenylethynyl)phenyl)-1,6-diazabicyclo[6.2.0]decane-6-carboxamide)

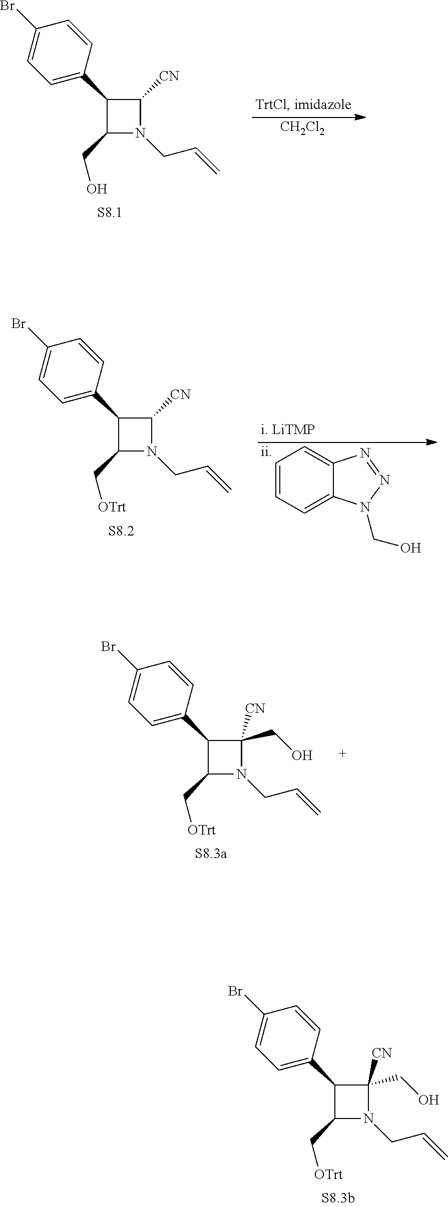

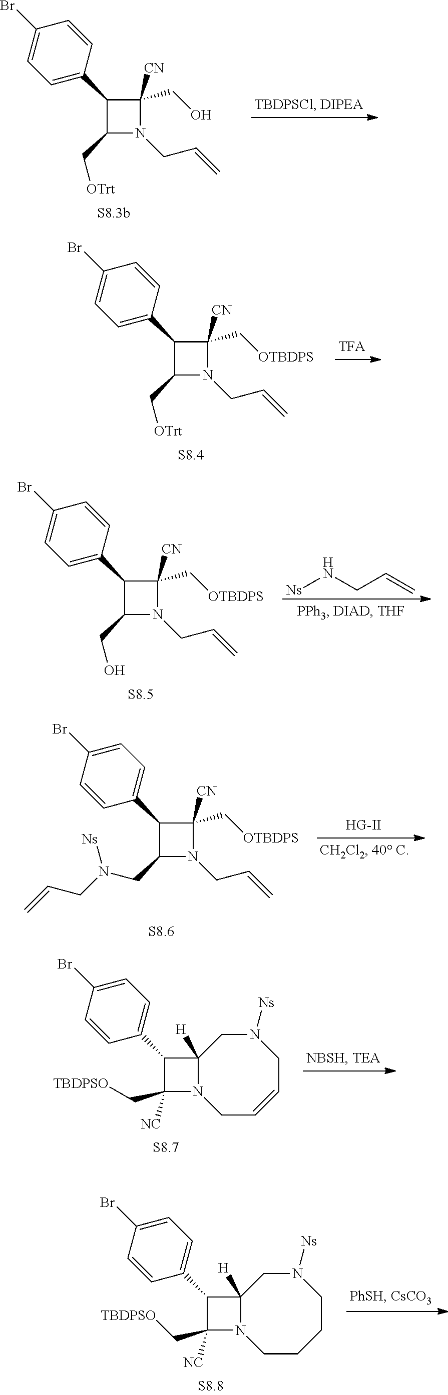

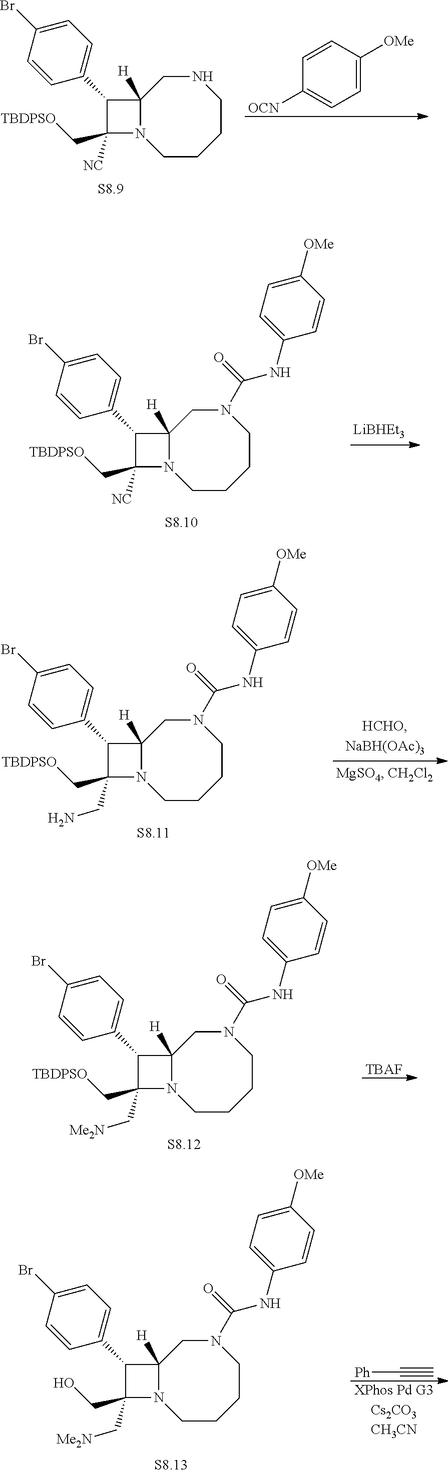

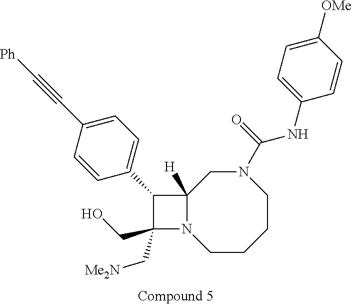 [SMILES: COc1ccc(NC(=O)N2CCCCN3[C@@H](C2)[C@@H](c2ccc(C#Cc4ccccc4)cc2)[C@@]3(CO)CN(C)C)cc1]

### (2R,3S,4R)-1-allyl-3-(4-bromophenyl)-4-((trityloxy)methyl)azetidine-2-carbonitrile (S8.2)

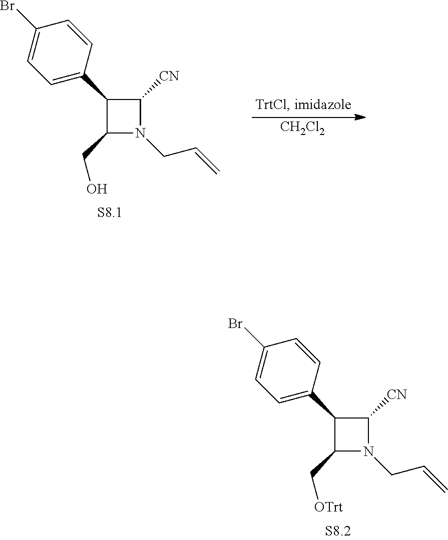

To a stirred solution of S8.1 (3.00 g, 9.77 mmol, 1.00 equiv)⁴ and triethylamine (1.98 g, 19.5 mmol, 2.71 mL, 2.00 equiv) in CH₂Cl₂ (30 mL) was added trityl chloride (3.27 g, 11.72 mmol, 1.20 equiv) at rt. The reaction mixture was stirred at rt for 1 h. LC-MS showed the reaction was complete. The reaction mixture was quenched with H₂O (1 mL) and extracted with CH₂Cl₂ (20 mL×3) to give the organic layer. The layer was dried over anhydrous Na₂SO₄ and concentrated. The residue was purified by column chromatography on silica gel (petroleum ether/EtOAc=1:1) to give 2 (6.00 g, crude) as light brown solid.

### (2R,3R,4R)- and (2R,3S,4R)-1-allyl-3-(4-bromophenyl)-2-(hydroxymethyl)-4-((trityloxy)methyl)azetidine-2-carbonitrile (S8.3a and S8.3b, respectively)

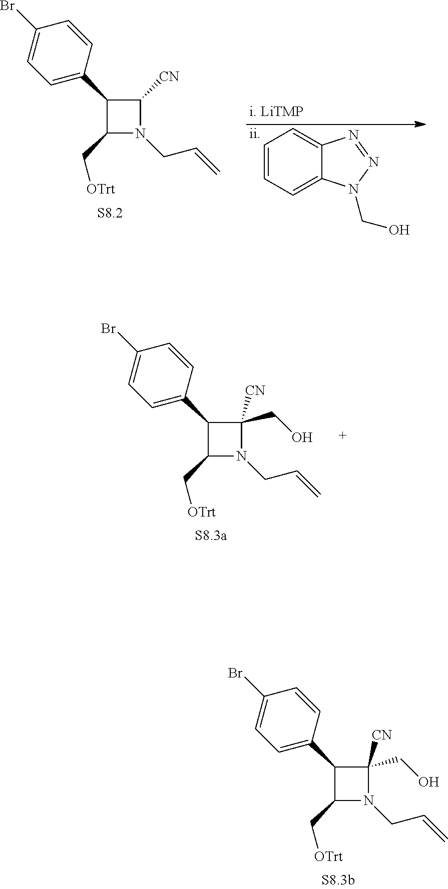

To a precooled (0° C.) solution of n-BuLi (2.5 M in hexane, 10.9 mL, 3.0 equiv) in THE (10 mL) was added tetramethylpiperidine (3.98 g, 28.2 mmol, 4.80 mL, 3.10 equiv). The reaction mixture was stirred at 0° C. for 0.5 h and cooled to −78° C. Then a solution of S8.2 (5.00 g, 9.10 mmol, 1.00 equiv) was added. The reaction mixture was stirred at −78° C. for 0.5 h. After 15 min, benzotriazol-1-ylmethanol (2.71 g, 18.20 mmol, 2.00 equiv) was added portionwise. The reaction mixture was stirred at −78° C. for 2 h, then warmed to rt. TLC (petroleum ether/EtOAc=2:1) showed the reaction was complete. The reaction mixture was concentrated. The residue was purified by column chromatography on silica gel (petroleum ether/EtOAc=2:1) to give S8.3a (1.12 g, 1.93 mmol, 21% yield) as light brown solid and S8.3b (3.11 g, 5.37 mmol, 59% yield) as light brown solid.

LC-MS m/z 581.1 (base peak) [M+H]⁺

¹H NMR (S8.3b) (400 MHz, CDCl₃) δ 7.36 (d, J=8.5 Hz, 2H), 7.27-7.20 (m, 11H), 7.14-7.16 (m, 6H), 5.77 (tdd, J=6.4, 10.3, 17.0 Hz, 1H), 5.18 (dd, J=1.4, 17.1 Hz, 1H), 5.04 (dd, J=1.3, 10.2 Hz, 1H), 4.04-4.01 (m, 3H), 3.82 (d, J=8.0 Hz, 1H), 3.52 (dd, J=6.1, 13.8 Hz, 1H), 3.24-3.20 (m, 2H), 2.96 (dd, J=7.7, 9.7 Hz, 1H).

### (2S,3R,4R)-1-allyl-3-(4-bromophenyl)-2-(((tert-butyldiphenylsilyl)oxy)methyl)-4-((trityloxy)methyl)azetidine-2-carbonitrile (S8.4)

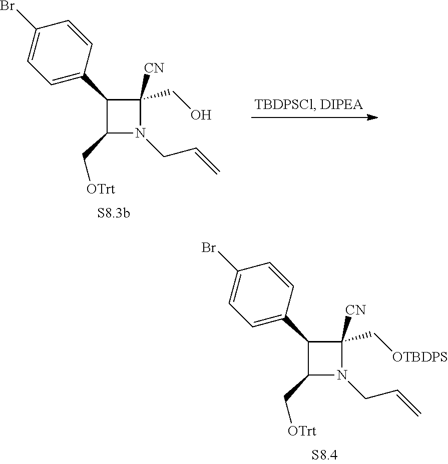

To a stirred solution of S8.3b (3.00 g, 5.18 mmol, 1.00 equiv) and imidazole (705 mg, 10.4 mmol, 2.00 equiv) in CH₂Cl₂ (5.0 mL) was added TBDPSCl (1.71 g, 6.21 mmol, 1.60 mL, 1.20 equiv). The reaction mixture was stirred at rt for 12 h. LC-MS showed the reaction was complete. The reaction mixture was quenched with H₂O (5 mL) and extracted with CH₂Cl₂ (10 mL×3) to give the organic layer. The layer was dried over anhydrous Na₂SO₄ and concentrate. The residue was purified by column chromatography on silica gel (petroleum ether/EtOAc=10:1) to give S8.4 (3.40 g, 4.16 mmol, 80% yield) as light brown solid.

LC-MS m/z 819.1 (base peak) [M+H]⁺

¹H NMR (400 MHz, CDCl₃) δ 7.69-7.61 (m, 4H), 7.46-7.27 (m, 8H), 7.20-7.09 (m, 11H), 7.07-7.01 (m, 6H), 5.60 (tdd, J=6.4, 10.3, 16.9 Hz, 1H), 4.98-4.80 (m, 2H), 4.03 (s, 2H), 3.99 (dt, J=5.0, 8.1 Hz, 1H), 3.63 (d, J=7.9 Hz, 1H), 3.45 (dd, J=6.3, 13.6 Hz, 1H), 3.15 (dd, J 6.5, 13.6 Hz, 1H), 3.01 (dd, J=5.0, 9.5 Hz, 1H), 2.78 (t, J=8.8 Hz, 1H), 1.07 (s, 9H).

### (2S,3R,4R)-1-allyl-3-(4-bromophenyl)-2-(((tert-butyldiphenylsilyl)oxy)methyl)-4-(hydroxymethyl)azetidine-2-carbonitrile (S8.5)

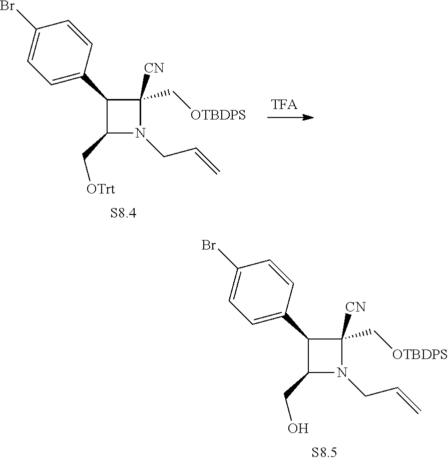

To a stirred solution of S8.4 (3.40 g, 4.16 mmol, 1.00 equiv) in CH₂Cl₂ (5.0 mL) was added TFA (4.74 g, 41.57 mmol, 3.1 mL, 10.0 equiv). The reaction mixture was stirred at rt for 12 h. LC-MS showed the reaction was complete. The reaction mixture was quenched with sat. aq. NaHCO₃ (10 mL) and extracted with CH₂Cl₂ (20 mL×3) to give the organic layer. The layer was dried over anhydrous Na₂SO₄ and concentrated. The residue was purified by column chromatography on silica gel (petroleum ether/EtOAc=3:1) to give S8.5 (1.88 g, 3.27 mmol, 79% yield) as light brown solid.

LC-MS m/z 577.0 [M+H]⁺

¹H NMR (400 MHz, CDCl₃) δ 7.68-7.57 (m, 4H), 7.49-7.28 (m, 10H), 5.79 (tdd, J=6.3, 10.4, 16.8 Hz, 1H), 5.18-5.02 (m, 2H), 4.00 (s, 2H), 3.85 (td, J=6.0, 8.3 Hz, 1H), 3.67 (d, J=8.4 Hz, 1H), 3.62-3.48 (m, 2H), 3.40-3.31 (m, 1H), 3.23 (dd, J=6.7, 13.9 Hz, 1H), 1.05 (s, 9H).

### N-allyl-N-(((2R,3R,4S)-1-allyl-3-(4-bromophenyl)-4-(((tert-butyldiphenylsilyl)oxy)methyl)-4-cyanoazetidin-2-yl)methyl)-4-nitrobenzenesulfonamide (S8.6)

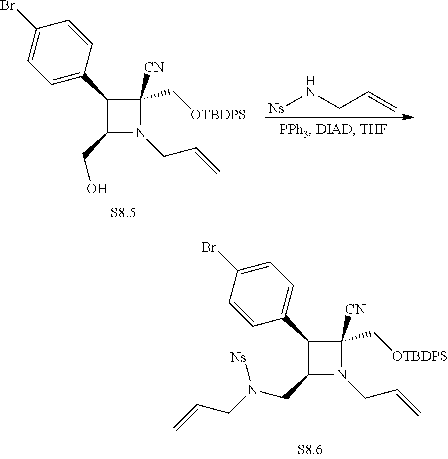

To a stirred solution of 5 (1.88 g, 3.27 mmol, 1.00 equiv), N-(o-nosyl)allylamine (820 mg, 3.59 mmol, 1.10 equiv) and PPh₃ (1.28 g, 4.90 mmol, 1.50 equiv) in THE (1.0 mL) was added DIAD (991 mg, 4.90 mmol, 0.95 mL, 1.50 equiv). The reaction mixture was stirred at rt for 12 h. LC-MS showed the reaction was complete. The reaction mixture was concentrated. The residue was purified by column chromatography on silica gel (petroleum ether/EtOAc=2:1) to give S8.6 (1.87 g, 2.34 mmol, 72% yield) as a white solid.

LC-MS m/z 801.1 (base peak) [M+H]⁺

¹H NMR (400 MHz, CDCl₃) δ 7.64 (ddd, J=1.6, 4.5, 7.8 Hz, 4H), 7.60-7.55 (m, 1H), 7.50-7.35 (m, 10H), 7.32-7.27 (m, 3H), 5.84-5.70 (m, 1H), 5.35 (tdd, J=6.2, 10.4, 16.8 Hz, 1H), 5.11-5.01 (m, 2H), 5.00-4.93 (m, 1H), 4.82 (d, J=17.1 Hz, 1H), 4.15-4.06 (m, 2H), 3.98 (d, J=11.0 Hz, 1H), 3.77-3.67 (m, 2H), 3.50-3.25 (m, 3H), 3.16-2.99 (m, 2H), 1.12-1.00 (m, 9H).

### (8R,9R,10S,Z)-9-(4-bromophenyl)-10-(((tert-butyldiphenylsilyl)oxy)methyl)-6-((4-nitrophenyl)sulfonyl)-1,6-diazabicyclo[6.2.0]dec-3-ene-10-carbonitrile (S8.7)

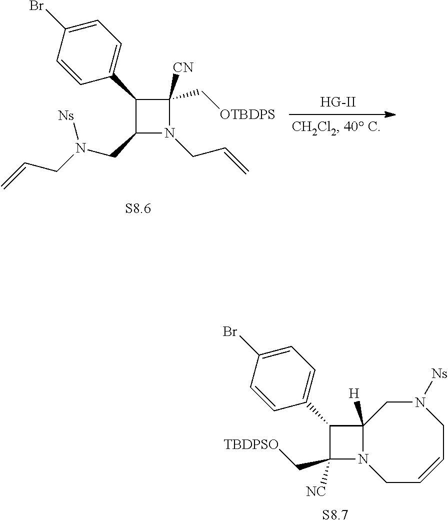

To a stirred solution of S8.6 (1.87 g, 2.34 mmol, 1.00 equiv) in CH₂Cl₂ (187 mL) was added Hoveyda-Grubbs 2^{nd} generation catalyst (367 mg, 585 μmol, 0.25 equiv). The reaction mixture was stirred at 40° C. for 12 h. LC-MS showed the reaction was complete. The reaction mixture was concentrated. The residue was purified by column chromatography on silica gel (petroleum ether/EtOAc=1:1) to give S8.7 (1.50 g, 1.94 mmol, 83% yield) as a light brown solid.

LC-MS m/z 773.1 (base peak) [M+H]⁺

¹H NMR (400 MHz, CDCl₃) δ 7.77 (dd, J=1.4, 7.6 Hz, 1H), 7.65-7.52 (m, 7H), 7.46-7.29 (m, 8H), 7.23 (d, J=8.3 Hz, 2H), 5.97-5.86 (m, 2H), 4.17-4.08 (m, 1H), 3.99-3.87 (m, 2H), 3.87-3.72 (m, 3H), 3.60 (dd, J=7.7, 12.2 Hz, 1H), 3.45-3.28 (m, 2H), 3.19 (dd, J=5.5, 12.2 Hz, 1H), 1.06-0.94 (m, 9H).

### (8R,9R,10S)-9-(4-bromophenyl)-10-(((tert-butyldiphenylsilyl)oxy)methyl)-6-((4-nitrophenyl)sulfonyl)-1,6-diazabicyclo[6.2.0]decane-10-carbonitrile (S8.8)

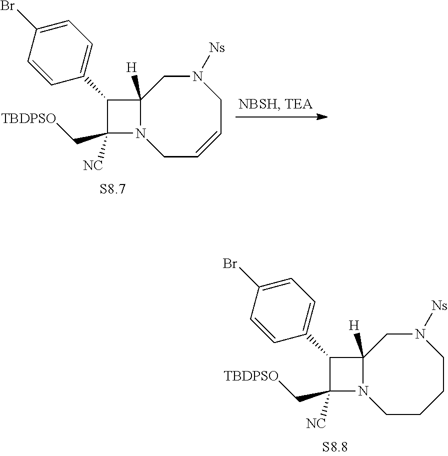

To a stirred solution of S8.7 (1.40 g, 1.81 mmol, 1.00 equiv) and Et₃N (1.84 g, 18.1 mmol, 2.51 mL, 10.0 equiv) in THE (1.0 mL) was added 2-nitrobenzenesulfonohydrazide (1.97 g, 9.07 mmol, 5.00 equiv). The reaction mixture was stirred at 40° C. for 12 h. LC-MS and TLC showed the reaction was complete. The reaction mixture was quenched with H₂O (2 mL) and extracted with CH₂Cl₂ (10 mL×3). The combined organic layers were dried over anhydrous Na₂SO₄ and concentrated. The residue was purified by preparative TLC to give S8.8 (1.33 g, 1.72 mmol, 95% yield) as a light brown solid.

LC-MS m/z 775.1 [M+H]⁺

¹H NMR (400 MHz, CDCl₃) δ 7.59-7.64 (m, 5H), 7.46-7.58 (m, 3H), 7.34-7.45 (m, 8H), 7.31 (d, J=8.53 Hz, 2H), 3.94-4.14 (m, 3H), 3.74 (ddd, J=3.83, 7.28, 14.49 Hz, 1H), 3.56 (d, J=8.41 Hz, 1H), 3.15 (br d, J=13.30 Hz, 1H), 3.03 (ddd, J=3.64, 8.72, 12.23 Hz, 1H), 2.65-2.94 (m, 3H), 1.86 (br d, J=9.66 Hz, 1H), 1.76 (br d, J=4.39 Hz, 2H), 1.63 (br s, 1H), 1.04 (s, 9H).

### (8R,9R,10S)-9-(4-bromophenyl)-10-(((tert-butyldiphenylsilyl)oxy)methyl)-1,6-diazabicyclo[6.2.0]decane-10-carbonitrile (S8.9)

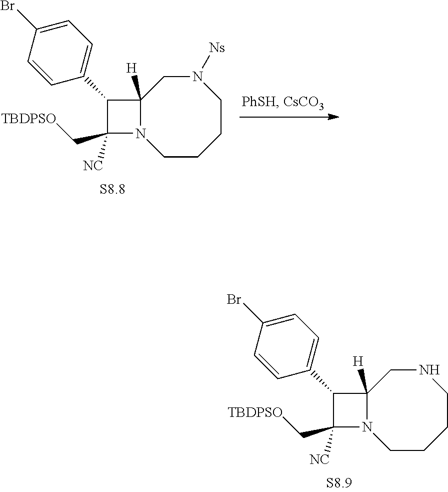

To a stirred solution of S8.8 (1.32 g, 1.71 mmol, 1.00 equiv) and Cs₂CO₃ (1.11 g, 3.42 mmol, 2.00 equiv) in CH₃CN (13.0 mL) was added benzenethiol (283 mg, 2.57 mmol, 262 μL, 1.50 equiv). The reaction mixture was stirred at rt for 12 h. TLC (CH₂Cl₂/CH₃OH=12:1) showed the reaction was complete. The reaction mixture was quenched with H₂O (20 mL) and extracted with CH₂Cl₂ (50 mL×3). The combined organic layers were dried over anhydrous Na₂SO₄ and concentrated. The residue was purified by column chromatography on silica gel (CH₂Cl₂/CH₃OH=12:1) to give S8.9 (955 mg, 1.62 mmol, 95% yield) as a light brown solid.

LC-MS m/z 590.1 (base peak) [M+H]⁺

¹H NMR (400 MHz, CDCl₃) δ 7.68-7.56 (m, 4H), 7.45-7.30 (m, 8H), 7.27 (d, J=8.4 Hz, 2H), 4.08-3.91 (m, 2H), 3.76 (dt, J=3.3, 8.3 Hz, 1H), 3.49 (d, J=8.3 Hz, 1H), 3.15-3.05 (m, 1H), 2.97-2.86 (m, 1H), 2.64-2.47 (m, 3H), 2.28 (dd, J=3.5, 14.4 Hz, 1H), 1.86-1.69 (m, 3H), 1.52-1.42 (m, 1H), 1.07-0.98 (m, 9H).

### (8R,9R,10S)-9-(4-bromophenyl)-10-(((tert-butyldiphenylsilyl)oxy)methyl)-10-cyano-N-(4-methoxyphenyl)-1,6-diazabicyclo[6.2.0]decane-6-carboxamide (S8.10)

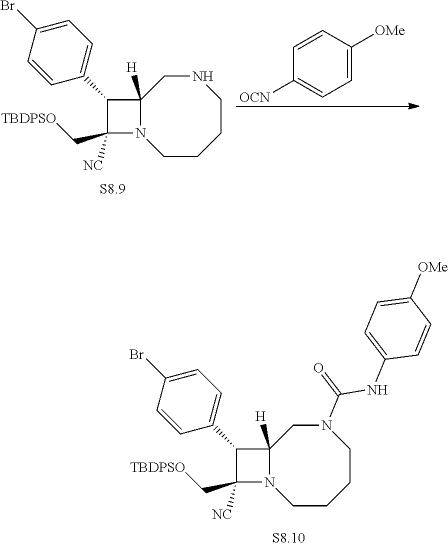

To a stirred solution of S8.9 (955 mg, 1.62 mmol, 1.00 equiv) and Et₃N (328 mg, 3.24 mmol, 450 μL, 2.00 equiv) in CH₂Cl₂ (1.0 mL) was added 1-isocyanato-4-methoxybenzene (266 mg, 1.78 mmol, 229 μL, 1.10 equiv). The reaction mixture was stirred at rt for 1 h. TLC (petroleum ether/EtOAc=2:1) showed the reaction was complete. The reaction mixture was quenched with H₂O (5 mL) and extracted with CH₂Cl₂ (20 mL×3). The combined organic layers were dried over anhydrous Na₂SO₄ and concentrated. The residue was purified by column chromatography on silica gel (petroleum ether/EtOAc=2:1) to give S8.10 (1.05 g, 1.42 mmol, 89% yield) as a light brown solid.

LC-MS m/z 739.2 [M+H]⁺

¹H NMR (400 MHz, CD₃OD) δ 7.73-7.69 (m, 4H), 7.57-7.52 (m, 2H), 7.52-7.39 (m, 8H), 7.18 (d, J=7.8 Hz, 2H), 6.82 (d, J=7.7 Hz, 2H), 4.24-4.06 (m, 2H), 4.06-3.94 (m, 2H), 3.75 (s, 3H), 3.66 (d, J=8.4 Hz, 1H), 3.45 (br d, J=13.5 Hz, 1H), 3.27-3.12 (m, 1H), 3.10-2.97 (m, 1H), 2.89 (br dd, J=10.4, 14.3 Hz, 1H), 2.78-2.65 (m, 1H), 1.91-1.63 (m, 4H), 1.08 (s, 9H).

### (8R,9R,10S)-10-(aminomethyl)-9-(4-bromophenyl)-10-(((tert-butyldiphenylsilyl)oxy)methyl)-N-(4-methoxyphenyl)-1,6-diazabicyclo[6.2.0]decane-6-carboxamide (S8.11)

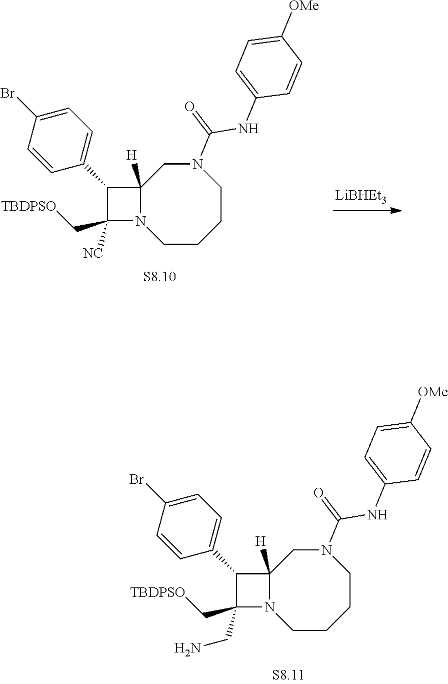

To a precooled (0° C.) solution of S8.10 (500 mg, 678 μmol, 1.00 equiv) in THE (10.0 mL) was added LiBHEt₃ (1 M, 6.78 mL, 10.0 equiv). The reaction mixture was warmed to rt and stirred for 1 h. LC-MS showed the reaction was complete. The reaction mixture was quenched with H₂O (2 mL) and extracted with CH₂Cl₂ (10 mL×3). The combined organic layers were dried over anhydrous Na₂SO₄ and concentrated to give S8.11 (1.00 g, crude) as a light brown solid.

LC-MS m/z 743.2 (base peak) [M+H]⁺

### (8R,9R,10S)-9-(4-bromophenyl)-10-(((tert-butyldiphenylsilyl)oxy)methyl)-10-((dimethylamino)methyl)-N-(4-methoxyphenyl)-1,6-diazabicyclo[6.2.0]decane-6-carboxamide (S8.12)

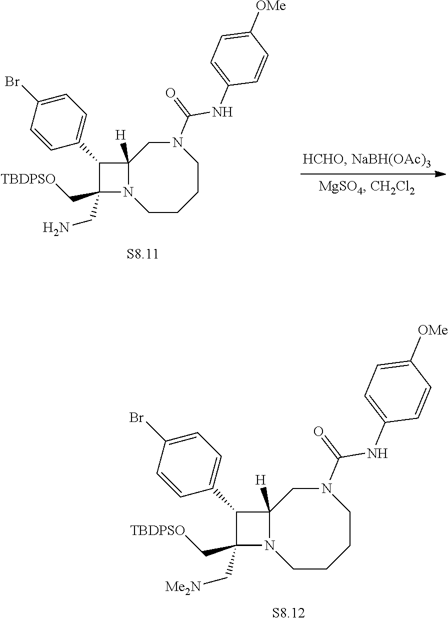

To a stirred solution of S8.11 (1.00 g, 1.35 mmol, 1.00 equiv) in CH₂Cl₂ (2.00 mL) were added formalin (formaldehyde 37% w/w in H₂O, 1.09 g, 13.48 mmol, 1.00 mL, 10.0 equiv) and MgSO₄ (1.62 g, 13.48 mmol, 10.0 equiv). The resulting reaction mixture was stirred at rt for 0.5 h. To the mixture were added acetic acid (81 mg, 1.35 mmol, 77 μL, 1.0 equiv) and NaBH(OAc)₃ (1.43 g, 6.74 mmol, 5.00 equiv). The resulting reaction mixture was stirred at rt for 1.5 h. TLC (CH₂Cl₂/CH₃OH=7:1) showed the reaction was complete. The reaction mixture was quenched with sat. aq. NaHCO₃ (5 mL) and extracted with CH₂Cl₂ (20 mL×3). The combined organic layers were dried over anhydrous Na₂SO₄ and concentrated. The residue was purified by preparative TLC (CH₂Cl₂/CH₃OH=7:1) to give S8.12 (394 mg, 512 μmol, 38% yield) as a white solid.

LC-MS m/z 771.2 (base peak) [M+H]⁺

¹H NMR (400 MHz, CDCl₃) δ 7.66 (dd, J=1.6, 7.7 Hz, 4H), 7.43-7.24 (m, 8H), 7.24-7.11 (m, 4H), 6.80-6.71 (m, 2H), 5.91 (s, 1H), 4.08-3.83 (m, 4H), 3.71 (s, 3H), 3.43-3.30 (m, 2H), 3.20-3.07 (m, 1H), 3.04-2.86 (m, 2H), 2.53-2.48 (m, 1H), 1.87-1.42 (m, 12H), 1.10-0.92 (m, 9H).

### (8R,9R,10 S)-9-(4-bromophenyl)-10-((dimethylamino)methyl)-10-(hydroxymethyl)-N-(4-methoxyphenyl)-1,6-diazabicyclo[6.2.0]decane-6-carboxamide (S8.13)

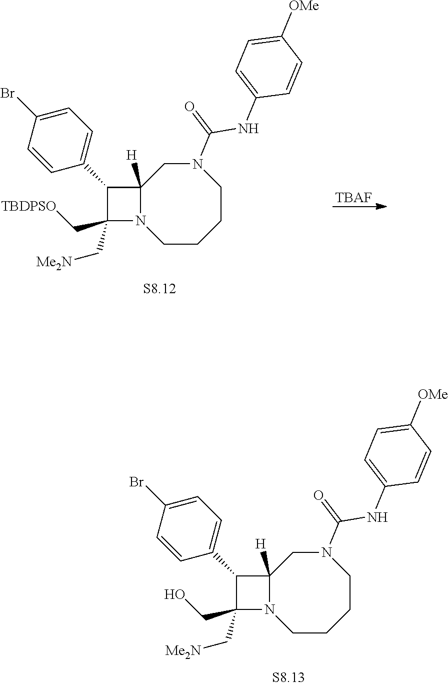

To a stirred solution of S8.12 (200 mg, 260 μmol, 1.00 equiv) in THE (1.0 mL) was added TBAF.3H₂O (164 mg, 520 μmol, 2.00 equiv). The resulting reaction mixture was stirred at rt for 12 h. LC-MS showed the reaction was complete. The reaction mixture was concentrated. The residue was purified by preparative TLC (CH₂Cl₂/CH₃OH=7.5:1) to give S8.13 (41 mg, 77 μmol, 30% yield) as a white solid.

LC-MS m/z 531.1 [M+H]⁺

¹H NMR (400 MHz, CDCl₃) δ 7.38 (d, J=8.4 Hz, 2H), 7.28 (d, J=7.5 Hz, 2H), 7.20-7.15 (m, 2H), 6.76 (d, J=9.0 Hz, 2H), 6.00 (s, 1H), 4.14 (d, J=11.7 Hz, 1H), 3.93-3.66 (m, 5H), 3.53 (br d, J=14.2 Hz, 1H), 3.35-3.22 (m, 1H), 3.21-3.10 (m, 1H), 3.00 (br dd, J=10.4, 15.6 Hz, 1H), 2.88-2.76 (m, 1H), 2.59 (d, J=13.9 Hz, 1H), 2.47-2.33 (m, 2H), 1.78 (s, 6H), 1.68-1.21 (m, 4H).

### (8R,9R,10S)-10-((dimethylamino)methyl)-10-(hydroxymethyl)-N-(4-methoxyphenyl)-9-(4-(phenylethynyl)phenyl)-1,6-diazabicyclo[6.2.0]decane-6-carboxamide (Compound 5)

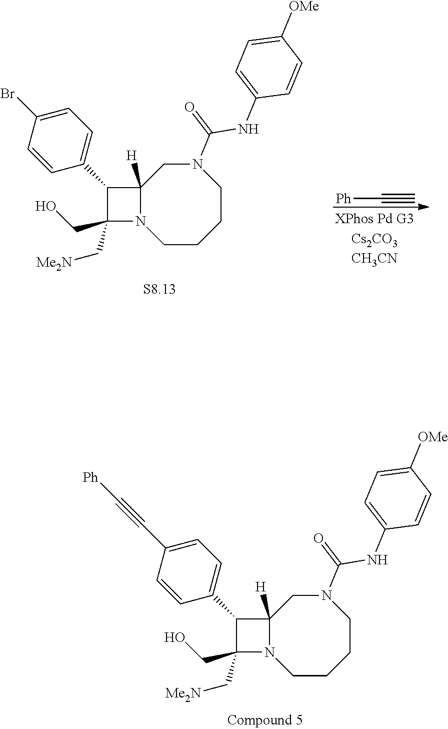

To a solution of S8.13 (35.0 mg, 65.6 μmol, 1.00 equiv) and ethynylbenzene (20.2 mg, 198 μmol, 21.70 μL, 3.00 equiv) in CH₃CN (1.00 mL) were added XPhos 3^{rd} generation precatalyst (5.6 mg, 6.6 μmol, 0.10 equiv) and Cs₂CO₃ (43 mg, 132 μmol, 2.0 equiv). The resulting reaction mixture was stirred at 70° C. for 2 h. LC-MS showed the reaction was complete. The reaction mixture was quenched with H₂O (2 mL) and extracted with CH₂Cl₂ (10 mL×3). The combined organic layers were dried over anhydrous Na₂SO₄ and concentrated. The residue was purified by preparative TLC (CH₂Cl₂/CH₃OH=7:1) followed by preparative HPLC (buffer C) to the give Compound 5 (13.9 mg, 25.2 μmol, 38% yield) as a white solid.

LC-MS m/z 553.4 [M+H]⁺

¹H NMR (400 MHz, CD₃OD) δ 7.45-7.64 (m, 6H), 7.29-7.42 (m, 3H), 7.14-7.25 (m, 2H), 6.77-6.89 (m, 2H), 4.24 (d, J=11.69 Hz, 1H), 3.97-4.12 (m, 2H), 3.83-3.94 (m, 1H), 3.75 (s, 3H), 3.44-3.61 (m, 2H), 3.25 (br dd, J=6.28, 12.68 Hz, 1H), 2.95-3.16 (m, 2H), 2.68-2.90 (m, 2H), 2.52 (br dd, J=8.93, 12.90 Hz, 1H), 2.08 (s, 6H), 1.77-1.96 (m, 2H), 1.49-1.76 (m, 2H), 2 exchangeable protons not reported.

### Synthesis of Compound 2 ((8R,9S,10S)-8-(methoxymethyl)-N-(4-methoxyphenyl)-9-(4-(phenylethynyl)phenyl)-10-((trityloxy)methyl)-1,6-diazabicyclo[6.2.0]decane-6-carboxamide)

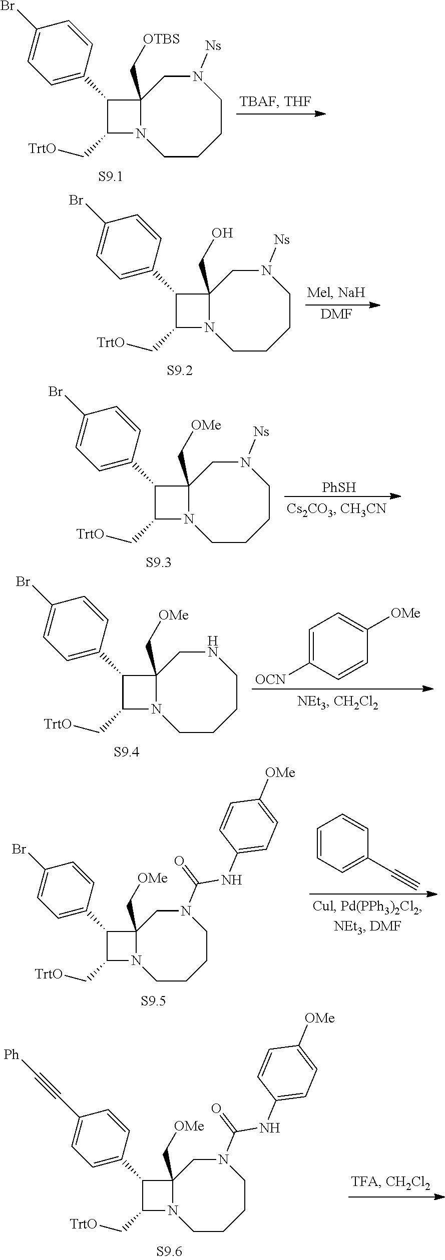

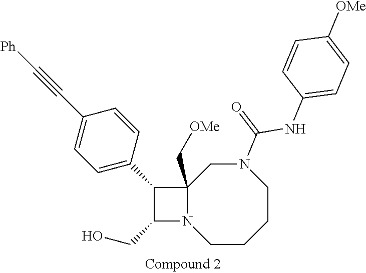 [SMILES: COC[C@@]12CN(C(=O)Nc3ccc(OC)cc3)CCCCN1[C@H](CO)[C@@H]2c1ccc(C#Cc2ccccc2)cc1]

### ((8R,9S,10S)-9-(4-bromophenyl)-6-((4-nitrophenyl)sulfonyl)-10-((trityloxy)methyl)-1,6-diazabicyclo[6.2.0]decan-8-yl)methanol (S9.2)

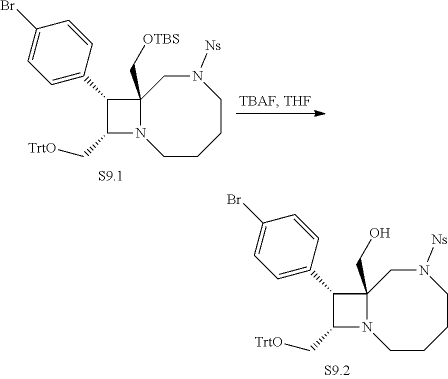

To a solution of S9.1 (200 mg, 223 μmol, 1.0 equiv) in THE (4.0 mL) was added TBAF (1.0 M, 446 μL, 2.0 equiv) and the mixture was stirred at 25° C. for 1 hr. TLC (Petroleum ether/EtOAc=3:1) showed the reaction was complete. The reaction was portioned between with brine (10 mL) and EtOAc (10 mL). The organic phase was washed with brine (10 mL×3), dried over anhydrous Na₂SO₄, filtered and concentrated to give S9.2 (180 mg, crude) as light yellow solid.

### (8R,9S,10S)-9-(4-bromophenyl)-8-(methoxymethyl)-6-((4-nitrophenyl)sulfonyl)-10-((trityloxy)methyl)-1,6-diazabicyclo[6.2.0]decane (S9.3)

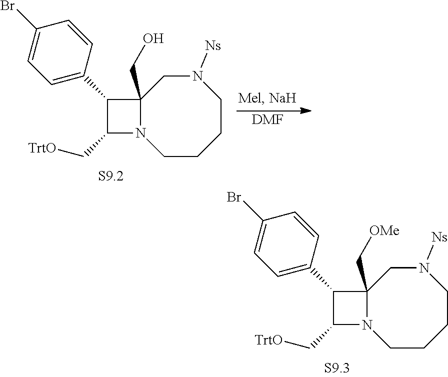

To a solution of S9.2 (180 mg, 230 μmol, 1.0 equiv) and Mel (39 mg, 276 μmol, 1.2 equiv) in DMF (6.0 mL) was added NaH (11 mg, 276 μmol, 60% w/w dispersion in mineral oil, 1.2 equiv) at −10° C. The mixture was slowly warmed to 25° C. and stirred for 12 hours. LC-MS showed the reaction was complete. H₂O (5 mL) was added, and the mixture was extracted with EtOAc (8 mL×3). The combined organic layers were washed with brine (8 mL×2), dried over anhydrous Na₂SO₄, filtered and concentrated. The residue was partially purified by preparative TLC (SiO₂, Petroleum ether/EtOAc=5:1) to give S9.3 (130 mg, crude) as a light yellow solid.

LC-MS m/z 796.3 [M+H]⁺

### (8R,9S,10S)-9-(4-bromophenyl)-8-(methoxymethyl)-10-((trityloxy)methyl)-1,6-diazabicyclo[6.2.0]decane (S9.4)

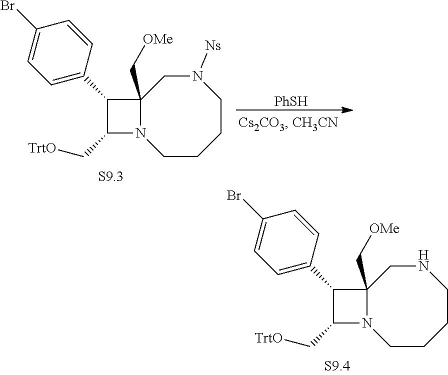

To a solution of S9.3 (130 mg, 163 μmol, 1.0 equiv) and Cs₂CO₃ (266 mg, 816 μmol, 5.0 equiv) in CH₃CN (10 mL) was added benzenethiol (108 mg, 979 μmol, 6.0 equiv) at 0° C. The mixture was stirred at 25° C. for 12 hours, at which point TLC (EtOAc) showed the reaction was complete. The reaction mixture was diluted with H₂O (20 mL) and extracted with EtOAc (8 mL×2). The organic phase was dried over anhydrous Na₂SO₄, filtered and concentrated. The residue was partially purified by preparative TLC (EtOAc) to give S9.4 (100 mg, crude) as a light yellow solid.

### (8R,9S,10S)-9-(4-bromophenyl)-8-(methoxymethyl)-N-(4-methoxyphenyl)-10-((trityloxy)methyl)-1,6-diazabicyclo[6.2.0]decane-6-carboxamide (S9.5)

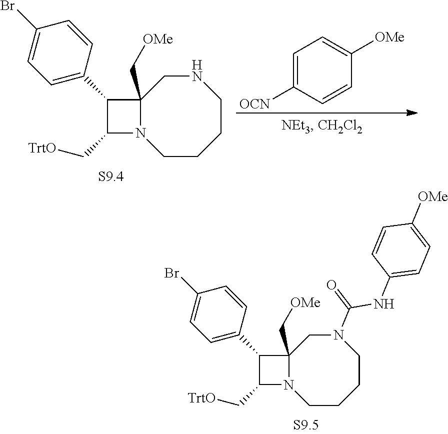

To a solution of S9.4 (100 mg, 164 μmol, 1.0 equiv) in CH₂Cl₂ (8 mL) was added triethylamine (16.5 mg, 163.5 μmol, 1.0 equiv) and 1-isocyanato-4-methoxy-benzene (29 mg, 196 μmol, 1.2 equiv). The mixture was stirred at 25° C. for 0.5 hours, at which point LC-MS showed the reaction was complete. The reaction mixture was concentrated and partially purified by preparative TLC (SiO₂, Petroleum ether/EtOAc=3:1) to give S9.5 (110 mg, crude) as a light yellow solid.

LC-MS m/z 762.4 (base peak) [M+H]⁺

### (8R,9S,10S)-8-(methoxymethyl)-N-(4-methoxyphenyl)-9-(4-(phenylethynyl)phenyl)-10-((trityloxy)methyl)-1,6-diazabicyclo[6.2.0]decane-6-carboxamide (S9.6)

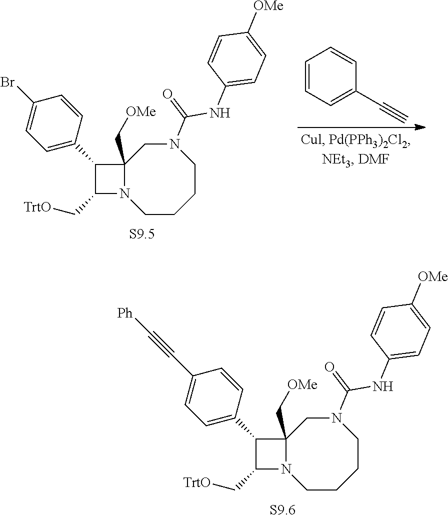

To a solution of S9.5 (30 mg, 40 μmol, 1.0 equiv) in DMF (2.0 mL) and triethylamine (1.0 mL) were added ethynylbenzene (12 mg, 120 μmol, 3.0 equiv), CuI (0.8 mg, 4 μmol, 0.10 equiv) and Pd(PPh₃)₂Cl₂ (2.8 mg, 3.9 μmol, 0.1 equiv). The tube reactor was sealed and heated to 100° C. (microwave) for 3 hours, at which point LC-MS showed the desired product was formed. The mixture was concentrated and partially purified by preparative TLC (SiO₂, Petroleum ether/EtOAc=5:1) to give S9.6 (30 mg, crude) as light yellow oil.

LC-MS m/z 782.6 [M+H]⁺

### (8R,9S,10S)-8-(methoxymethyl)-N-(4-methoxyphenyl)-9-(4-(phenylethynyl)phenyl)-10-((trityloxy)methyl)-1,6-diazabicyclo[6.2.0]decane-6-carboxamide (Compound 2)

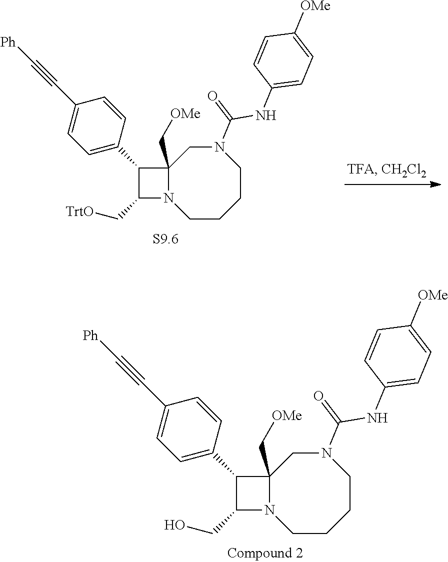

To a solution of S9.6 (30 mg, 39 μmol, 1.0 equiv) in CH₂Cl₂ (2.0 mL) was added TFA (60 mg, 0.53 mmol, 14.0 equiv). The mixture was stirred at 30° C. for 0.5 hours, at which point LC-MS deemed the reaction complete. The reaction mixture was concentrated and purified by preparative HPLC (buffer E) to give Compound 2 (7.3 mg, 11.5 μmol, 30% yield, 85% purity) as a yellow solid.

LC-MS m/z 540.2 [M+H]⁺

¹H NMR (400 MHz, CD₃OD) δ 7.53-7.41 (m, 6H), 7.39-7.28 (m, 3H), 7.22-7.12 (m, 2H), 6.86-6.74 (m, 2H), 4.12 (br d, J=14.3 Hz, 1H), 3.98-3.76 (m, 4H), 3.74-3.66 (m, 5H), 3.61-3.53 (m, 1H), 3.50-3.45 (m, 3H), 3.24-3.17 (m, 1H), 3.13-3.02 (m, 1H), 2.90 (br s, 1H), 2.78-2.66 (m, 1H), 1.84-1.58 (m, 4H), 2 exchangeable protons not observed.

Activity Measurements

### Example 9: Cell-Based Assay for Anticryptosporidial Screening and Potency Measurement

A high-content microscopy assay was used to measure activity of compounds against C. parvum grown in the human adenocarcinoma cell line HCT-8 (ATCC) as disclosed in Bessoff, K., et al., Antimicrobial agents and chemotherapy 57, 1804-1814, doi:10.1128/AAC.02460-12 (2013), hereby incorporated by reference in its entirety. HCT-8 cells were cultured in 384-well clear-bottomed plates in 50 μL per well of RPMI 1640 medium (Invitrogen) supplemented with 10% heat-inactivated fetal bovine serum (Sigma-Aldrich), 120 U·mL⁻¹ penicillin, and 120 μg·mL⁻¹ streptomycin (ATCC) at 37° C. under 5% CO₂. For compound screening and follow-up SAR studies, confluent cell monolayers were infected with C. parvum Iowa isolate oocysts [Bunch Grass Farm (Deary, Id.); ˜5,500 oocysts per well] after inducing excystation by treatment for 10 min at 37° C. with 10 mM HCl and then 10 min at 16° C. in 2 mM sodium taurocholate. Compounds were added at the indicated concentrations 3 h after monolayer infection. After incubation (37° C., 5% CO₂) for 48 h, the cells were washed three times with PBS containing 111 mM D-Galactose and fixed by adding an equal volume of 8% formaldehyde in PBS. Cell monolayers were then prepared for microscopy by permeabilizing with 0.1% Triton X-100, washing three times with PBS with 0.1% Tween 20, blocking with 4% bovine serum albumin (BSA) in PBS for 2 h at 37° C. or 4° C. overnight, and staining parasitophorous vacuoles with 1.33 μg·mL⁻¹ of fluorescein-labeled Vicia villosa lectin (Vector Laboratories) diluted in 1% BSA in PBS with 0.1% Tween 20 for 1 h at 37° C. DNA was then stained by adding Hoechst 33258 (AnaSpec) at a final concentration of 0.09 mM diluted in water, followed by washing five times with PBS containing 0.1% Tween 20. Images were acquired using a Nikon Eclipse TE2000 epifluorescence microscope with an automated stage that was programmed using NIS-Elements Advanced Research software (Nikon) to focus on the center of each well and take a 3×3 composite image using an EXi Blue fluorescence microscopy camera (QImaging) with a 20× objective (numerical aperture 0.45). Nucleus and parasite images were exported separately as tif files and analyzed using NIH ImageJ and previously reported macros, and data analysis and graphing were done using GraphPad Prism software, version 6.01.

Studies using farm C. parvum isolates and the C. hominis TU502 isolate were performed by Calibr at Scripps Research (La Jolla, Calif.) in 1,536-well plates using a slightly modified immunofluorescence assay as described in Love, M. S. et al. PLoS neglected tropical diseases 11, e0005373, doi:10.1371/journal.pntd.0005373 (2017), hereby incorporated by reference in its entirety. HCT-8 cell culture medium was replaced with RPMI 1640 medium supplemented with 2% heat-inactivated horse serum, 100 U·mL⁻¹ penicillin, and 100 mg·mL streptomycin 24 h prior to infection with the indicated isolate. The C. parvum Iowa isolate was included as a reference in all experiments. Imaging was performed using a Cellnsight CX5 High Content screening platform (Thermo Scientific) with a 10× objective and acquisition of one microscopic field per well. Images were processed using HCS Studio Scan software, and the selected-object count (HCT-8 cells) and spot count (parasitophorous vacuoles) were analyzed in Genedata Screener (version 13.0; Standard). Dose-response curves and EC₅₀ values were calculated using the Smart Fit function of Genedata Analyzer.

### Example 10: DNA Replication and Sexual Differentiation Assays

DNA synthesis was assayed during intracellular development of C. parvum (Iowa isolate) within HCT-8 cells by measuring incorporation of the thymidine analogue 5-ethynyl-2′-deoxyuridine (EdU) using epifluorescence microscopy as described in Jumani, R. S. et al. Nat Commun 10, 1862, doi:10.1038/s41467-019-09880-w (2019), hereby incorporated by reference in its entirety. HCT-8 cells were grown to >90% confluence in 96-well plates coated with 50 μg mL⁻¹ fibronectin (BD Pharmingen, catalog #354008), and then infected using the method described above with ˜5.5×10⁴ C. parvum Iowa isolate oocysts per well. Compound 11 was added at 2×EC₉₀ 3 hours after infection, followed by incubation for 6 hours and then addition of 10 mM EdU. After incubation for two more hours, the monolayers were washed and fixed with 4% formaldehyde in saline. Cells were then permeabilized, and stained with FITC-Vicia villosa lectin, Hoechst 33258, and for incorporation of EdU using the Click-iT assay kit (Thermo Fisher Scientific). Images were acquired by focusing on the parasite focal plane on top of the host cell monolayer using a 40× objective (0.7 NA), and parasitophorous vacuoles and EdU positive vacuoles were quantified using NIH ImageJ software.

The effect on expression of the meiotic recombination protein DMC1 was previously identified as a sexual stage-specific marker for C. parvum. The potency of Compound 11 for inhibiting asexual development during the first 48 h of infection was compared to its potency at inhibiting DMC1 expression when compound was added after 48 h of culture (the approximate timing of sexual differentiation). In addition to staining for parasites with FITC-Vicia villosa lectin and DNA with Hoechst as above, samples were stained for the presence of DMC1 with an anti-C. parvum DMC1 mouse monoclonal antibody (clone 1H10G7 (IgG2b, kappa) used as undiluted culture medium with a secondary Alexa Fluor 568 goat anti-mouse IgG antibody (Invitrogen) at 1:500 dilution. Cultures were performed in 384-well plates with the addition of amphotericin B (Sigma, catalog) to the culture medium at 0.1 to 0.5 μg·mL⁻¹ for these assays. Images were acquired as above, and analyzed using NIH ImageJ software.

### Example 11: C. parvum Time-Kill Curve Assay

Time-kill curve assays were performed in 384-well plates using C. parvum Iowa isolate oocysts (Bunch Grass Farms, Deary, Id.) following infection of HCT-8 cell monolayers as described in Jumani, R. S. et al. Antimicrobial agents and chemotherapy 62, e01505-01517 (2018), hereby incorporated by reference in its entirety. The methods for HCT-8 cell infection, staining host cell nuclei and parasites, and image acquisition were identical to those described above, except that Compound 11 (EC₅₀ or multiples of the EC₉₀) or vehicle was added after 24 h of culture followed by incubation for variable lengths of time. Single-phase exponential decay curves were fit using GraphPad Prism software version 6.01 after normalizing the percentage of host cells infected to the vehicle control value at each time point, which isolates the effect of compound treatment from the spontaneous reduction in parasite numbers that occurs in this culture system.

### Example 12: Mouse Model of Established Cryptosporidiosis

NOD scid gamma mice (NOD.Cg-Prkdcscid Il2rgtmlWjl/Szj; Jackson Laboratory, Bar Harbor, Me.) (NSG) were used to model established Cryptosporidium infection. All NSG mouse studies were performed in compliance with animal care guidelines and with approval by the University of Vermont Institutional Animal Care and Use Committee. Mice were acquired at three to four weeks of age, and acclimatized for one week prior to infection by oral gavage of ˜10⁵ C. parvum Iowa isolate (Bunch Grass Farms) oocysts. They were then treated at the specified times and dosages with each compound dissolved in 0.5% hydroxypropylmethyl cellulose, 0.5% Tween 80 (v/v) plus 5% DMSO. Fecal parasite shedding was measured using a quantitative PCR (qPCR) assay, using the 18S rRNA primers shown in Table 2.

TABLE 2

| Region | Primer name | Sequence (5′ to 3′) |
|---|---|---|
| 18S rRNA | Cp18s forward | TAGAGATTGGAGGTTGTTCCT |
| | Cp18s reverse | CTCCACCAACTAAGAACGGCC |
| Guide | Guide_PheS_F | GTTGGGCAATCACAACGATGTCAG |
| Cloning | Guide_PheS_R | AAACCTGACATCGTTGTGATTGCC |
| PheS_wt_ | PheS_wt_Avr_F | CTAGGCTGACATCGTTGTGATTGCA |
| fragment | | TGGGGGCTTTCTCTTGAAAGGCCAA |
| cloning | | CGATGATTAGTTATAATATCCCAAA |
| | | TATACGCGATCTATTTAGCTTTAAA |
| | | GCAAAAATATTTTCATAAGG |
| | PheS_wt_Asc_R | CGCGCCTTATGAAAATATTTTTGCT |
| | | TTAAAGCTAAATAGATCGCGTATAT |
| | | TTGGGATATTATAACTAATCATCGT |
| | | TGGCCTTTCAAGAGAAAGCCCCCAT |
| | | GCAATCACAACGATGTCAGC |
| PheS_mut_ | PheS_mut_Avr_F | CTAGGCTGACATCGTTGTGATTGCA |
| fragment | | TGGGGGGTTTCTCTTGAAAGGCCAA |
| cloning | | CGATGATTAGTTATAATATCCCAAA |
| | | TATACGCGATCTATTTAGCTTTAAA |
| | | GCAAAAATATTTTCATAAGG |
| | PheS_mut_Asc_R | CGCGCCTTATGAAAATATTTTTGCT |
| | | TTAAAGCTAAATAGATCGCGTATAT |
| | | TTGGGATATTATAACTAATCATCGT |
| | | TGGCCTTTCAAGAGAAAGCCCCCAT |
| | | GCAATCACAACGATGTCAGC |
| Overhang | ohgF_PheS_N_ | GTTGGAAATTCCGGCTTATTTAGAC |
| PCR | wmpam | CAGAAATGCTTAGACCACTCGGGTT |
| Repair | | TCCATCTGACATCGTTGTGATTGC |
| DNA | E ohg_pheR: | AGTGATTATAATCTAAAAAACAAA |
| | | CATTAATTCGAAAAGTCTACATTAG |
| | | GAATTAAGATAAAAAGAAAAAC |
| 5′ | P1 | CGACAAGAATCTTACACTTGCAGAT |
| integra- | | CTAATCGGAAC |
| tion | P2 | CTTTGGATCGGAGTTACGGACAC |
| 3′ | P3 | CAGACTTAAGGCCCGTATGCCCGAC |
| integra- | | GGTGAAGATCTTGTC |
| tion | P4 | CGAACCCGAGCCATCAGTTCGGAA |
| | | AACTGAGATGCTAGC |

### Example 13: Construction of Plasmids and DNA Repair

To generate the Cas9/guide plasmid, the pheS guide sequence was cloned using complementary oligonucleotides (Guide_PheS_F and Guide_PheS_R) with overhang sequences to anneal into the BbsI restriction sites of the Aldo-Cas9-ribo vector. For the repair construct, a 113 bp fragment of the C. parvum pheRS gene (cgd3_3320) spanning the region containing the wild type or desired mutation till the end of the gene (1418-1530 bp) was amplified using complementary oligonucleotides (PheS_wt_Avr_F, PheS_wt_Asc_R and PheS_mut_Avr_F, PheS_mut_Avr_R). The annealed product was cloned into AvrII/AscI sites of the vector Cplic3HAENNE, upstream of the Eno-Nluc-Neo-eno cassette of vector. Linear repair DNA containing 50 bp regions of homology on both ends and PAM change along with the Eno-Nluc-Neo-eno cassette was generated by overhang PCR using oligonucleotides ohgF_PheS_N_wmpam and ohg_pheR. Sequences of all primers are listed in Supplementary information, Table 2.

### Example 14: Mouse Infection to Create and Passage Transgenic Strains

All mouse studies described in this section were approved by the Institutional Animal Care and Use Committee of the University of Georgia and University of Pennsylvania. Cryptosporidium parvum IOWA-II oocysts were purchased from Bunch Grass Farms, Deary, Id., USA. Oocysts were excysted and electroporated with Cas9/guide plasmid and repair DNA template using Lonza Nucleofector 4D device and previously optimized protocols described in Vinayak, S. et al. Nature 523, 477-480, doi:10.1038/nature14651 (2015) and Pawlowic, et al. Curr Protoc Microbiol 46, 20B 22 21-20B 22 32, doi:10.1002/cpmc.33 (2017), each hereby incorporated by reference in their entirety. C57BL/6 IFN-γ knockout mice (Jackson Laboratory) aged 4-6 weeks (n=5) were infected with transfected sporozoites suspended in PBS using surgery described previously or an oral gavage procedure. The oral gavage procedure involved buffering of the acidic stomach with 8% sodium bicarbonate before delivery of sporozoites.

Fecal samples were collected from cages and luminescence measurements were performed as described in Manjunatha, U. H. et al. Nature 546, 376-380, doi:10.1038/nature22337 (2017) and Vinayak, S. et al. Nature 523, 477-480, doi:10.1038/nature14651 (2015), each hereby incorporated by reference in their entirety. Oocysts were purified from fecal material using sucrose flotation followed by cesium chloride purification. Purified oocysts were used to infect new cages of IFN-γ knockout mice to passage the transgenic strains and collect more oocysts for downstream experiments. During passaging, mice were monitored for weight loss, fur ruffling, hunched posture, and inactivity. Mice showing a weight loss of equal to or greater than 15% were euthanized.

### Example 15: PCR and Sequencing of Transgenic Oocysts

We performed PCR on fecal DNA to confirm the correct 5′ and 3′ integration events after homologous recombination. DNA was isolated from 100 mg of fecal material using a ZR Fecal DNA Miniprep Kit (Zymo Research). Fecal samples were subjected to five rounds of freeze-thawing, and then DNA was isolated following manufacturer's instructions. PCR was performed on fecal DNA isolated from wild type and mutant transgenic strains using primers P1, P2 and P3, P4 (sequences listed in Table 2) to confirm the correct integration events. The 5′ integration PCR amplification product from both wildtype and mutant transgenic strains were excised from the gel and cloned into TOPO cloning vector (Promega). Ten independent clones were picked for each strain, and isolated plasmids were sent for Sanger sequencing using M13F primer to confirm the wildtype leucine or valine mutation at the endogenous locus.

### Example 16: Cryptosporidium In Vitro Drug Assays and EC₅₀ Determination

The in vitro drug susceptibility assay using transgenic parasites expressing Nluc was performed as described in Vinyak et al. Briefly, host intestinal epithelial adenocarcinoma (HCT-8) cells grown in 96-well plates were infected with purified transgenic Nluc-expressing oocysts (1,000 oocysts per well) and incubated with different concentrations of Compound 11 for 48 hours. Culture supernatant was discarded from the wells after 48 hours, and 200 μl of NanoGlo lysis buffer containing 1:50 of NanoGlo substrate (Promega) was added to the wells. Lysates were transferred to white 96-well plates and luminescence was measured on a Glomax luminescence reader (Promega). EC₅₀ values were calculated in GraphPad Prism software v7.

### Example 17: C. hominis PheRS Biochemical Assay

The protein sequences of C. hominis PheRS alpha (Chro.30378) and beta subunits (Chro.80385) were retrieved from the Crypto DB (https://cryptodb.org/cryptodb/). Genes were inserted to pETM 11 and pETM 20 vectors and both alpha and beta subunits were co-expressed in E. coli B834 competent cells. Recombinant proteins were expressed by growing cells at 37° C. to OD₆₀₀ of 0.6-0.8, followed by induction with 0.6 mM isopropyl β-d-1-thiogalactopyranoside (IPTG). The cells were later harvested by centrifugation at 5,000×g and cell pellet suspended in a buffer containing 50 mM Tris-HCl (pH 8.8), 200 mM NaCl, 4 mM [3-mercaptoethanol, 10% (v/v) glycerol, 0.1 mg ml-1 lysozyme and 1 mM phenylmethylsulfonyl fluoride (PMSF)]. The bacterial cells were lysed using sonication and cleared by centrifugation at 20,000×g for 45 mins. The cleared supernatant was loaded on prepacked Ni-NTA column (GE Healthcare) and bound protein eluted using a concentration gradient of imidazole from 0 to 1 M in the buffer containing 50 mM Tris-HCl (pH 7.5), 80 mM NaCl, 4 mM [0-mercaptoethanol, 15% (v/v) glycerol, 1 M imidazole, using AKTA-FPLC system (GE healthcare]. Then, size exclusion chromatography was done using the GE HiLoad 60/600 Superdex column in buffer containing 50 mM Tris-HCl (pH 8), 200 mM NaCl, 4 mM (β-mercaptoethanol, and 1 mM MgCl₂ Protein purity was verified by SDS PAGE and protein aliquots were stored at the concentration of 4 mg/ml at −80° C. until further use.

Aminoacylation assays were done as described in Cestari, I. et al. Journal of Biomolecular Screening 18, 490-497, doi:10.1177/1087057112465980 (2013) and Sharma, A. et al. Biochemical Journal 465, 459-469, doi:10.1042/bj20140998 (2015), each hereby incorporated by reference in their entirety. These assays were performed in a buffer containing 30 mM HEPES (pH 7.5), 150 mM NaCl, 30 mM KCl, 50 mM MgCl₂, 1 mM DTT, 100 μM ATP, 50 μM L-phenylalanine, 2 U·mL⁻¹ E. coli inorganic pyrophosphatase (NEB), and 100 nM recombinant PheRS at 37° C. Inhibition assays were performed by using drug concentrations ranging from 0.0001 μM to 100 μM in the assay buffer.

### Example 18: Mouse Pharmacokinetics Methods

Pharmacokinetic analyses of Compounds 15, 12, and 9 were performed by Shanghai ChemPartner Co., Ltd Shanghai, 201203, P.R. China following single intravenous and oral administrations to female CD1 mice. Compounds 9 and 20 were formulated in 70% PEG400 and 30% (5% glucose in H₂O) for IV and PO dosing. Test compounds were dosed as a bolus solution intravenously (IV) at 0.6 mg/kg (dosing Solution; 70% PEG400 and 30% (5% glucose in H₂O) or dosed orally (PO) by gavage as a solution at 1 mg/kg (dosing Solution; 70% PEG400 and 30% (5% glucose in H₂O) to female CD1 mice (n=9/dose route). Pharmacokinetic parameters were estimated by non-compartmental model using WinNonlin 6.2.

Pharmacokinetic analyses of Compounds 13, 16, 10, 17, and 11 were performed by Eisai Inc. Andover, Mass. 01810, in male CD-1 mice using standard methods. Compounds 13, 16, 10, 17, and 11 were formulated in 10% ethanol, 4% Tween, 86% saline for both IV and PO dosing. PK parameters were estimated by a non-compartmental model using proprietary Eisai software.

### Example 19: Compound Screen

Screening was performed in duplicate at two concentrations (1 and 5 μM), and compounds demonstrating reproducible, dose-dependent inhibition of >30% were selected for follow-up testing. Cryptosporidium growth inhibition was confirmed using freshly supplied compounds and nine-point dose-response assays. The screen identified inhibitors of numerous potential targets, including the cytochrome bc1 complex, heat-shock protein 90 (HSP90), histone deacetylases (HDAC), phosphatidyl-inositol-4-kinase (PI4K), and multiple tRNA-synthetases. The most potent inhibitor identified was a Broad Institute DOS collection bicyclic azetidine, Compound 19, which had an EC₅₀ of 62 nM against the Bunch Grass Farm C. parvum Iowa strain. The bicyclic azetidine series disclosed herein was therefore prioritized for further studies.

### Example 20: Bicyclic Azetidines Potently Inhibit the Growth of Multiple Cryptosporidium Species and Strains

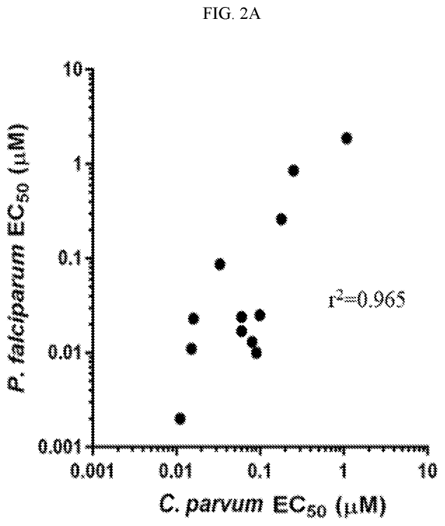

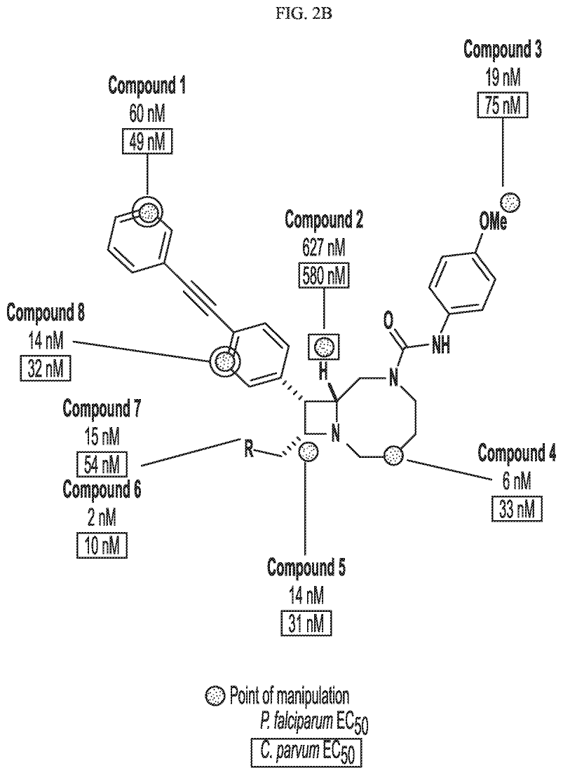

An initial set of 12 bicyclic azetidines was selected to evaluate compound activities against C. parvum. Activities of compounds were compared with compound activity on P. falciparum parasites. A positive correlation between C. parvum and P. falciparum activity (r²=0.965; FIGS. 2A and 2B) was found.

Nine analogues, each representing a different chemical manipulation at a key position, showed comparable shifts in growth inhibition of P. falciparum and C. parvum. In particular, Compound 2, an analogue in which the C4 position of the bicyclic azetidine is chemically manipulated, shows similarly reduced potency in P. falciparum (EC₅₀=627 nM) and C. parvum (EC₅₀=580 nM) (FIG. 2B, the boxed value is the EC₅₀ for the indicated compound against C. parvum growth).

Anti-Cryptosporidium activity of a subset of potent antimalarial compounds that varied in up to three positions on the compound structure and represented distinct chemotypes within the series (e.g. dibasic at physiological pH such as Compound 11, zwitterionic at physiological pH such as Compound 10 and monobasic at physiological pH such as Compound 17), each with different PK/PD properties, was measured. These bicyclic azetidines showed broad activity irrespective of the C. parvum isolate tested, and had comparable potencies against C. hominis and C. parvum (e.g. Compound 11: C. parvum (1% FBS) EC₅₀=9 nM, C. hominis EC₅₀=10 nM, Tables 3 and 4).

The cytotoxicity of these six bicyclic azetidines was analyzed as well and a selectivity index of >100 was determined for all compounds, comparing the C. parvum Iowa isolate (1% FBS) EC₅₀ to the half-maximal cytotoxicity concentration (CC₅₀) of HepG2 cells. In particular Compound 16, which was highly effective in vivo (see below), has a selectivity index of >10,000. The measured values for each compound having the structure:

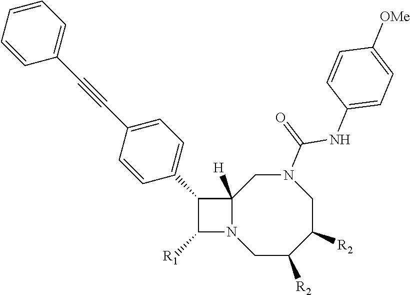

are shown in Tables 3 and 4.

TABLE 3

| Compound No. | 11 | 10 | 17 |
|---|---|---|---|
| R₁ | —CH₂N(Me)₂ | —CH₂O(CH₂)₂CO₂H | —CH₂N(Me)SO₂Me |
| R₂ | —H | —H | —H |
| C. parvum (Iowa) (1% | 9 | 4 | 6 |
| FBS) BGF EC₅₀ (nM) | | | |
| C. parvum (Iowa) (10% | 73 | 15 | 7 |
| FBS) BGF EC₅₀ (nM) | | | |
| C. parvum (Iowa) Sterling | 8 | 2 | 5 |
| EC₅₀ (nM) | | | |
| C. parvum (Field Isolate) | 23 | 9 | 6 |
| WSU EC₅₀ (nM) | | | |
| C. hominis (TU 502) | 10 | 5 | 4 |
| EC₅₀ (nM) | | | |
| HepG2 CC₅₀ (nM) | 5 × 10³ | >40 × 10³ | >40 × 10³ |
| HEK293T CC₅₀ (nM) | 5 × 10³ | >40 × 10³ | >40 × 10³ |

TABLE 4

| Compound No. | 16 | 18 | 19 |
|---|---|---|---|
| R₁ | —CH₂N(Me)C(O)Me | —CH₂SO₂Me | —H |
| R₂ | —H | —OH | —H |
| C. parvum (Iowa) (1% | 2 | <0.4 | 36 |
| FBS) BGF EC₅₀ (nM) | | | |
| C. parvum (Iowa) (10% | 8 | n.d. | 62 |
| FBS) BGF EC₅₀ (nM) | | | |
| C. parvum (Iowa) Sterling | 0.6 | <0.4 | 31 |
| EC₅₀ (nM) | | | |
| C. parvum (Field Isolate) | 6 | <0.4 | 96 |
| WSU EC₅₀ (nM) | | | |
| C. hominis (TU 502) | 3 | 2 | 55 |
| EC₅₀ (nM) | | | |
| HepG2 CC₅₀ (nM) | 25 × 10³ | 8 × 10³ | 10 × 10³ |
| HEK293T CC₅₀ (nM) | 27 × 10³ | 6 × 10³ | 25 × 10³ |

### Example 21: Bicyclic Azetidines are Curative in an Immunocompromised Mouse Model of Cryptosporidiosis

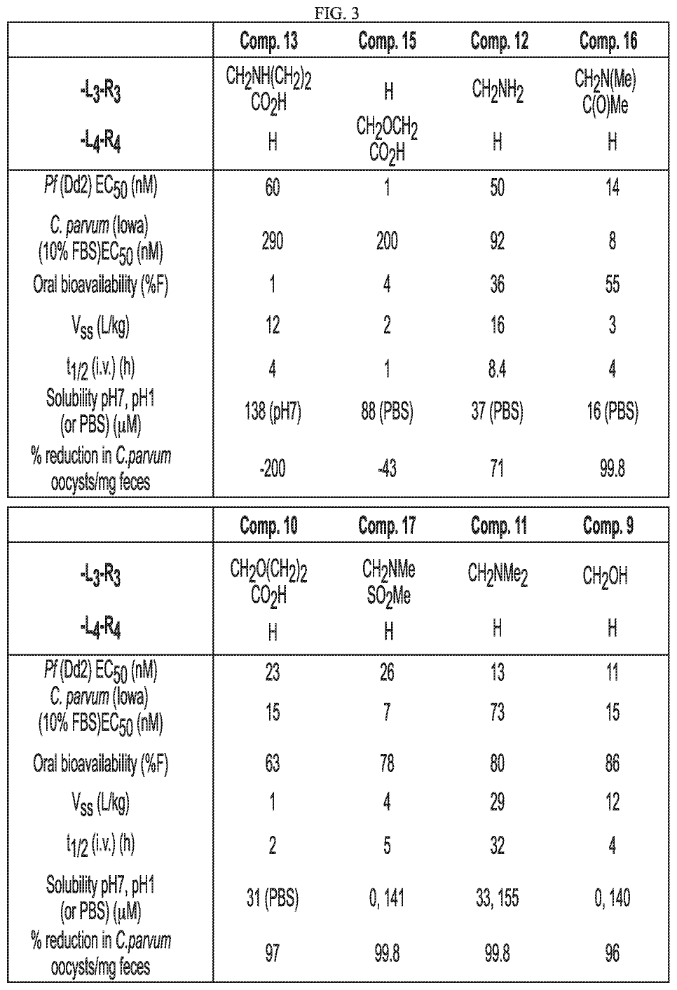

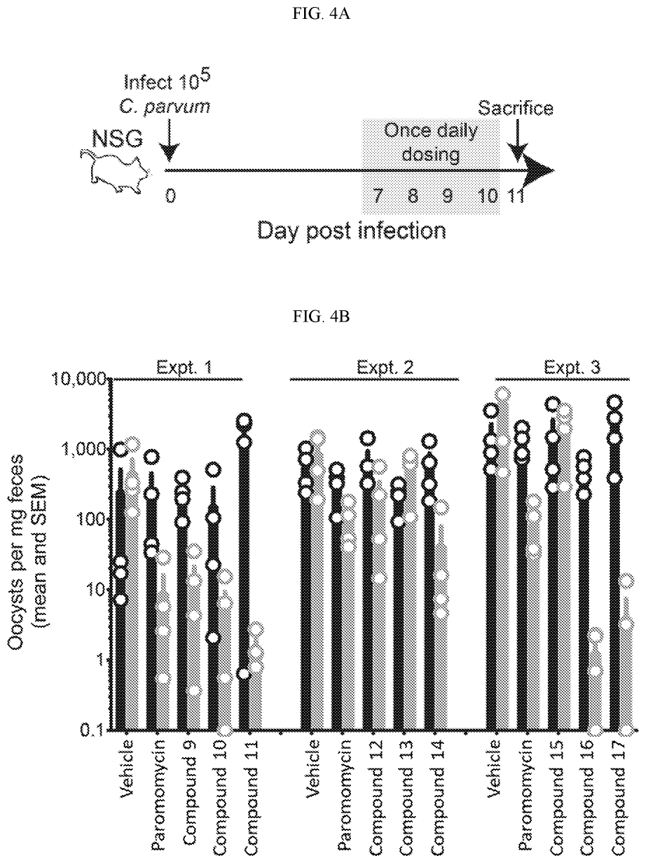

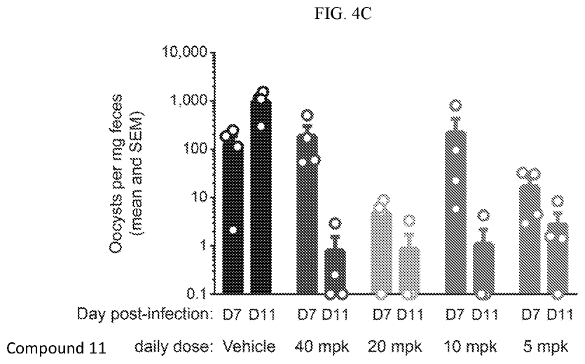

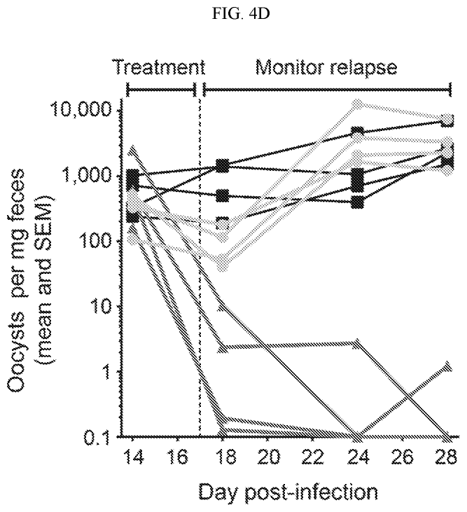

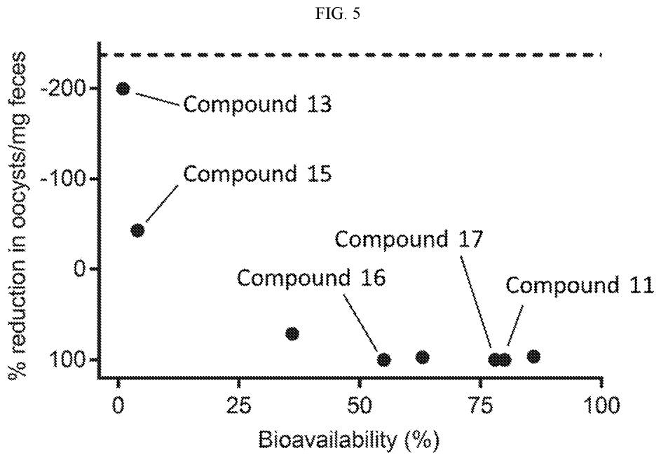

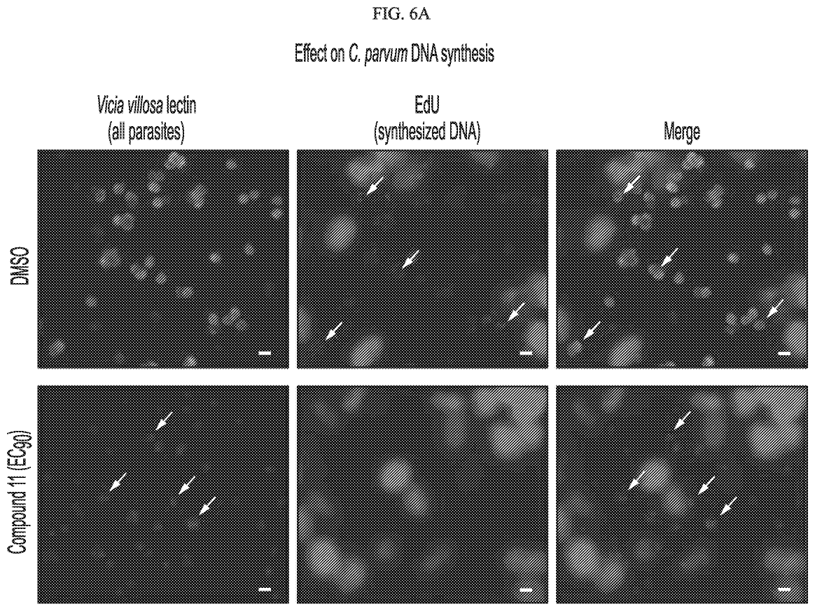

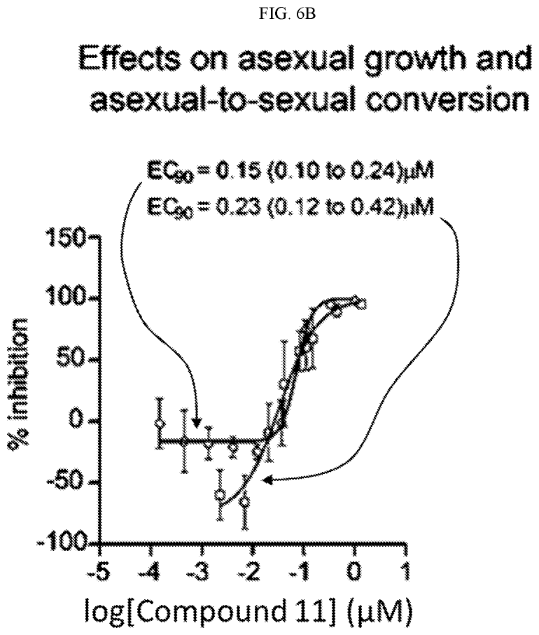

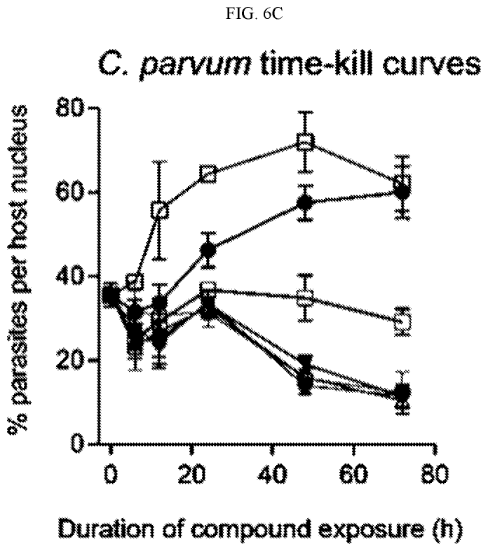

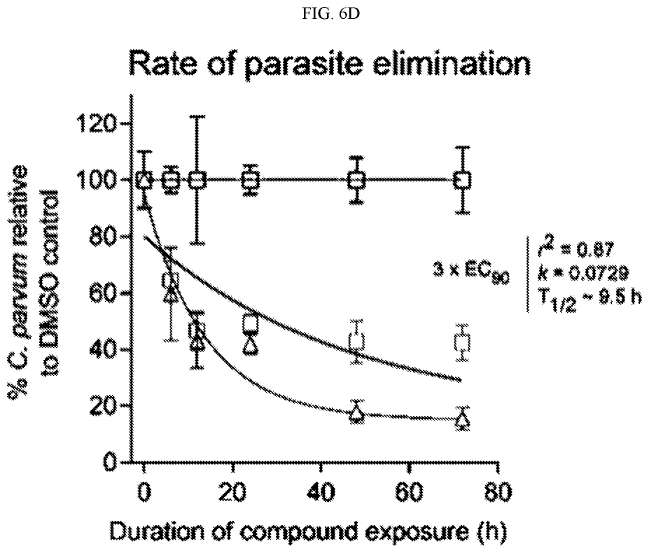

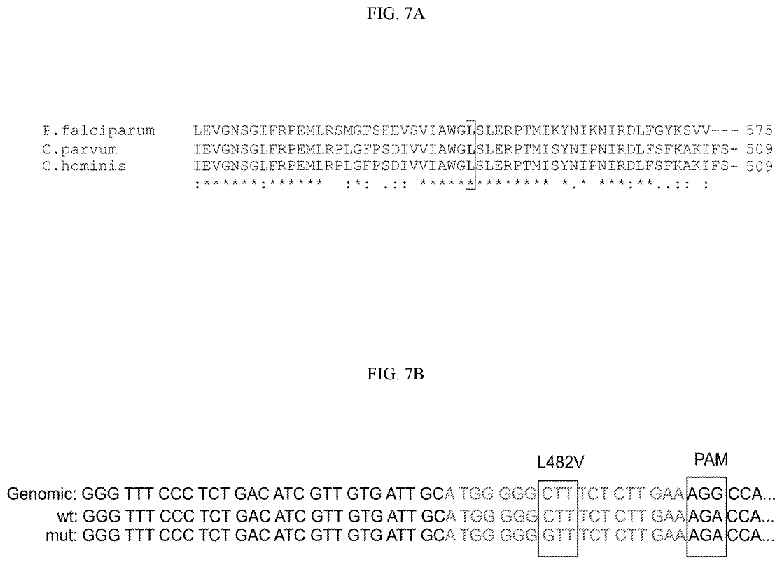

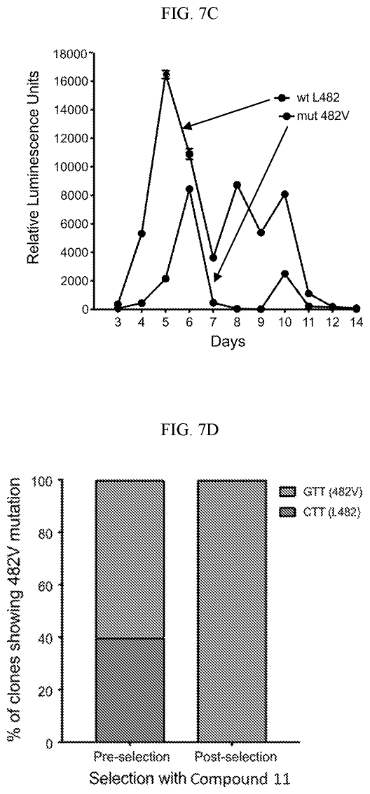

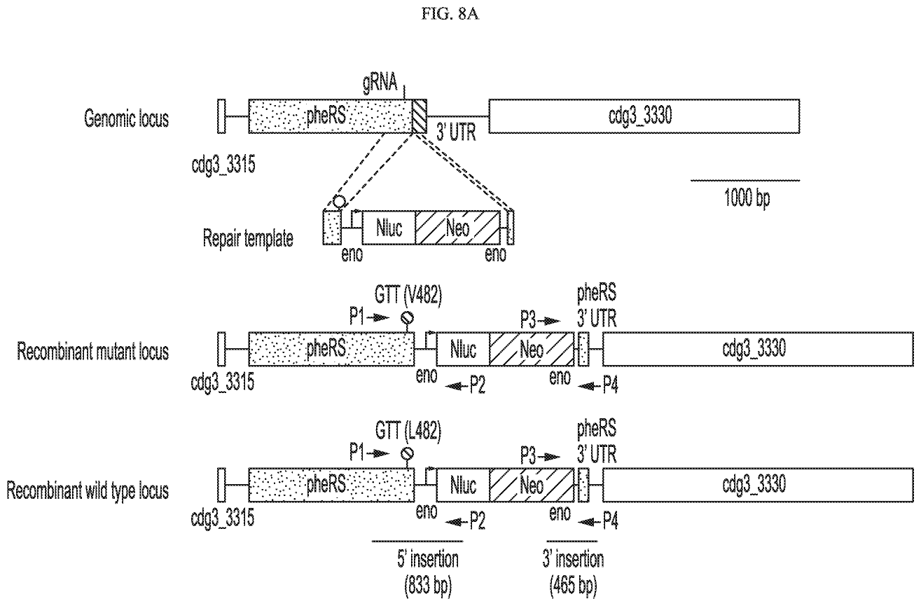

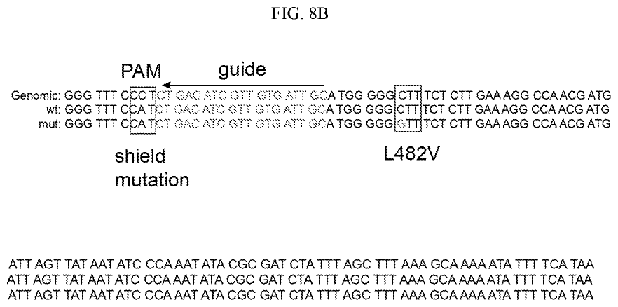

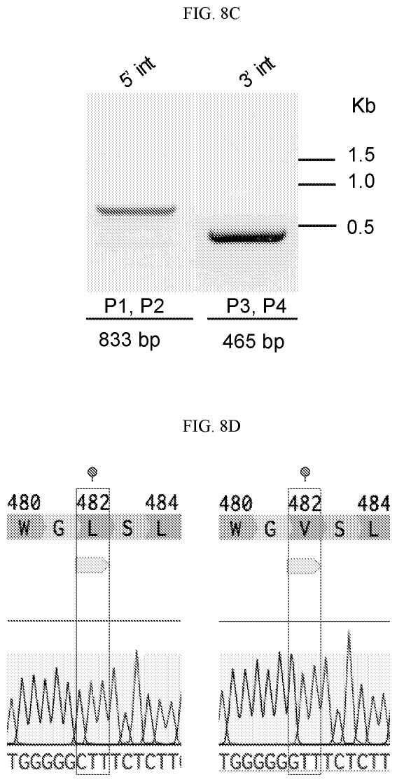

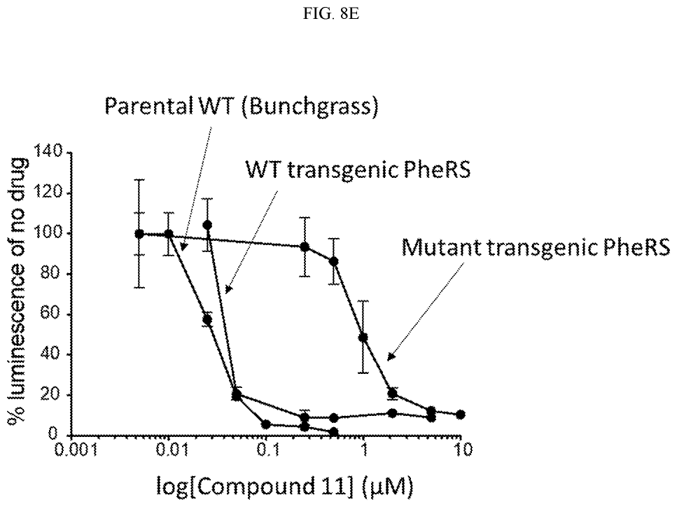

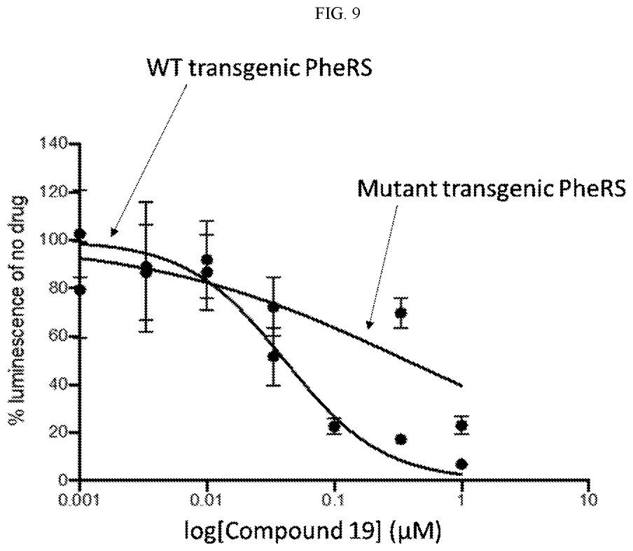

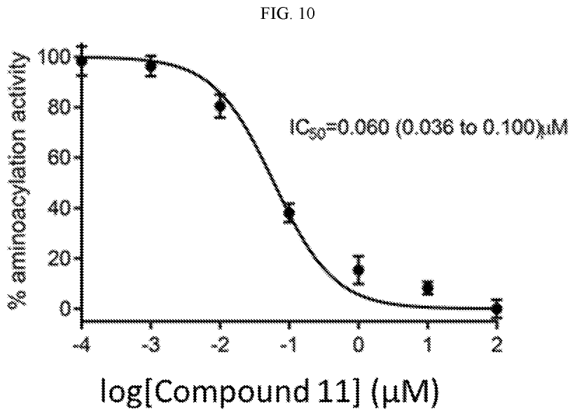

Three compounds (Compound 11, Compound 10 and Compound 9 with C. parvum EC₅₀<73 nM) representing three different chemotypes within the series (dibasic, zwitterion, and monobasic, respectively), and distinct PK profiles (e.g. half-life 2-32 h, Vₛₛ 1-29 L·kg⁻¹, see FIG. 3), were selected for testing in an immunocompromised mouse model of established C. parvum infection (FIG. 2b, exptl). This cryptosporidiosis infection model used 3-4 week old, freshly weaned, NOD SCID gamma mice orally infected with C. parvum oocysts (˜1×10⁵). The infection was monitored by qPCR of feces with primers specific for C. parvum and data are shown as number of oocysts milligram⁻¹ of feces. Six days following infection and upon confirmation of parasite shedding by qPCR, the three compounds, negative and positive control were orally administered at 50 mg·kg⁻¹ (body weight) every 24 h for 4 days. (FIGS. 4A-D). All three bicyclic azetidine compounds showed significant reduction in oocyst shedding. In particular, Compound 11 showed an almost 3-log reduction in oocysts·mg⁻¹ stool. We then performed a dose-ranging study with Compound 11 using the same immunocompromised mouse model and dosing regimens of 5, 20 and 40 mg·kg⁻¹ every 24 h for 4 days or 10 mg·kg⁻¹ every 24 h for 7 days. Mice were monitored for an additional 14 days for relapse of infection. At doses as low 10 mg·kg⁻¹ of Compound 11 every 24 h for 7 days, mice were apparently cured and no oocyst shedding was observed after 14 days (FIGS. 4C and D), further validating the series for drug development.

### Example 22: Bicyclic Azetidines with High Bioavailability Show Increased In Vivo Efficacy

The primary site of Cryptosporidium infection is the intestinal epithelium, and good luminal exposure is important for in vivo efficacy. While lowering systemic exposure may improve the safety margin of a cryptosporidiosis drug, there is a concern that gastrointestinal-only exposure could lead to drug washout in the watery diarrhea associated with the disease and may not be optimal for treatment of infection of the biliary epithelium. There are examples of compounds with both low and high systemic exposure in development for the treatment of cryptosporidiosis. Eight compounds were identified (including Compound 11, Compound 10, and Compound 9) with bioavailabilities ranging from 1-86% that maintained similar in vitro growth inhibition against C. parvum (EC₅₀<300 nM), thus allowing us to systematically investigate the ideal PK/PD profile for this series. These compounds were synthetically scaled-up and tested in the C. parvum immunocompromised mouse model. Azetidines with higher bioavailability (Compounds 11, 17 and 16, see FIGS. 3 and 5) showed the greatest reduction in oocysts mg⁻¹ feces, as assessed by qPCR of feces using C. parvum specific primers. An anti-Cryptosporidium agent needs to traverse at least three membrane barriers, suggesting that permeability is an important attribute for a drug candidate. It is therefore possible that the correlation of efficacy with bioavailability in our series reflects permeability differences between compounds with high and low bioavailability. Attempts to determine in vitro permeability by measuring apical to basolateral transport in a pig kidney cell line (LLC-PK1) were not successful due to poor compound recovery. While bicyclic azetidines with lower bioavailability were less potent than those with higher bioavailability, we note that all had robust anticryptosporidiosis activity (C. parvum EC₅₀<300 nM), suggesting reasonable cellular permeability under these conditions.

### Example 23: Bicyclic Azetidine Compound 11 Blocks Intracellular Development and Rapidly Kills C. parvum

The anticryptosporidial action of Compound 11 was further examined in vitro using a series of phenotypic assays roughly based on the Cryptosporidium life cycle. No effect was seen in an assay for host cell invasion. Compound 11 strongly inhibited intracellular parasite development, as assessed using a fluorescence microscopy-based assay that measures incorporation of the thymidine analogue 5-ethynyl-2′-deoxyuridine (EdU) into newly synthesized DNA (FIG. 6A). It should be noted that EdU incorporation in this assay is simply a surrogate for continued intracellular parasite development, and that this result does not indicate direct inhibition of DNA synthesis by Compound 11. Consistent with inhibition of CpPheRS by Compound 11 as in P. falciparum, the same effect has been reported with a number of anticryptosporidial aaRS-tRNA synthetase inhibitors. Compound 11 inhibition of C. parvum sexual development was compared to inhibition of asexual growth by comparing its effects on asexual growth during the first 48 hours of culture vs. expression of a sexual development biomarker, the meiotic recombination protein DMC1, between hours 48 and 72 of culture. As has been observed with other Cryptosporidium aaRS inhibitors, Compound 11 was approximately equipotent in both assays (FIG. 6B).

Successful treatment of immunocompromised patients such as AIDS patients, transplant recipients, and malnourished children might be anticipated to require a drug that rapidly eliminates parasites in the absence of significant help from the immune system. Indeed, NOD SCID gamma (NSG) mice infected with C. parvum quickly relapse or show no improvement at all when treated with the slow-acting anticryptosporidials paromomycin and clofazimine, respectively, despite the efficacy of both of these compounds in less severely immunocompromised mice. Consistent with its observed efficacy in the NSG mouse model, Compound 11 rapidly eliminated C. parvum from in vitro cultures, with a maximum rate of parasite elimination first achieved at a concentration 3×EC₉₀ (FIG. 6C). A similar effect has been reported with the piperazine-based anticryptosporidial MMV665917, which is curative in the NSG mouse model. To control for the spontaneous decline in parasite numbers that occurs after ˜60 hours of C. parvum culture in the HCT-8 cell system, these data were normalized to parasite numbers present at each time point for vehicle control samples. in vitro parasite elimination in the presence of Compound 11 was exponential with a half-life of ˜9.5 hours and ˜95 hours required to eliminate 99.9% of parasites (FIG. 6D).

### Example 24: CRISPR-Cas9 Mediated Single Base Editing of Cryptosporidium parvum PheRS

In vitro resistance selection experiments and whole genome sequencing performed previously for P. falciparum identified point mutations in PheRS that confer resistance to the bicyclic azetidine Compound 20. A key mutation at amino acid 550 of PheRS (leucine to valine; L550V) was identified in multiple independent P. falciparum clones that showed resistance to the bicylic azetidine series. Searching the genomes of numerous apicomplexan parasites available through EuPathDB (http://eupathdb.org) using the BLAST algorithm revealed homologs of PfcPheRS in all apicomplexan parasites, including C. parvum (cgd3_3320) and C. hominis. Importantly, the region with the critical PfcPheRS L550 residue at its center is conserved and shows a high degree of sequence similarity (FIG. 7A).

C. parvum sporozoites were electroporated with a suitable Cas9/guide plasmid as well as DNA repair templates containing homology arms with CTT (wild type) or GTT (mutation), along with a shield mutation (AGG to AGA) in the protospacer adjacent motif (PAM) (FIG. 7B). Electroporated sporozoites were used to infect groups of IFN-γ knockout mice, and stable transgenic parasites were selected with paromomycin. PCR amplification of parasite DNA and sequencing of the transgenic population revealed a mixture of wild type (CTT) and mutant (GTT) alleles (this is likely the consequence of the choice we made of Cas9 cleavage just 3′ of the mutation site). Cloning of the amplification product followed by sequencing of ten independent clones revealed that 40% of the clones were wild type and 60% harbored the desired mutation. This provided us with the opportunity of an unbiased selection experiment. IFN-γ knockout mice were infected (n=3 per group) with this mixed population of transgenic parasites (L482+V482, pink line) or with a control (wt L482, blue line), and treated with 10 mg·kg⁻¹ Compound 11 for five days (days 4-8 post-infection). Parasite shedding was monitored by measuring luminescence in the feces, and noted a decrease in luciferase activity after three days of drug treatment (FIG. 7C, day 7) in both strains but mixed population parasites rebounded markedly. The PheRS gene was amplified by PCR on day 8 and 11 following treatment, and sequenced eight independent clones from each day. In contrast to the input population, all sequences recovered were mutant at nucleotide position 1444 (corresponding to amino acid position 482) and the wild type allele was lost, suggesting that the mutation provided a selective advantage under Compound 11 treatment (FIG. 7D).

To further test the selective advantage, the guide RNA choice was altered to isolate parasites with an engineered PheRS locus that exclusively carried either an L or V residue at position 482 (FIGS. 8A-B). Note that both strains are genetically modified and identical in the remainder of the insertion. Stable transgenics were isolated by paromomycin selection in mice and PCR analysis confirmed correct integration into the PheRS locus (FIG. 8C, sequence of primers used are provided in Table 2). Sequencing of PCR products revealed uniform modification to mutant or wildtype and the presence of the synonymous shield mutation (FIG. 8D).

### Example 25: C. parvum Expressing the PheRS L482V Mutation are Resistant to Compound 11

Oocysts from the mutant (mut482V) and wild type (wtL482) transgenic lines were purified as well as the parent strain (Bunchgrass) used to generate the transgenics. Sporozoites were excised from these oocysts and used to infect 96 well format HCT-8 cultures to perform in vitro drug susceptibility assays. Cultures were incubated for 48 hours in the presence of varying concentrations of Compound 11 and parasite growth was measured by establishing whole well luciferase activity to calculate EC₅₀ values for each strain. The susceptibility of parent (EC₅₀=29 nM) and wild type transgenic (EC₅₀=47 nM) was indistinguishable. However, the mutant transgenic parasite showed a 23-fold decrease in Compound 11 susceptibility (EC₅₀=1059 nM, FIG. 8e). We also tested Compound 19 against wild type transgenic and mutant transgenic parasites, and found a 9-fold shift in EC₅₀ (wt transgenic EC₅₀=40.8 nM, mut transgenic EC₅₀=371.5 nM, FIG. 9). These results suggest that in C. parvum, as in P. falciparum, the PheRS 482V mutation causes profound resistance to bicyclic azetidines, thus providing strong genetic support for inhibition of CpPheRS as the MOA for the compounds disclosed herein.

### Example 26: Compound 11 Inhibits Purified Recombinant C. hominis PheRS in a Biochemical Assay

Recombinant C. hominis PheRS (ChPheRS) protein was also purified and measured its aminoacylation activity in the presence of a bicyclic azetidine as described previously with analogous enzymes. Compound 11 potently inhibited recombinant ChPheRS in a concentration-dependent manner (IC₅₀=60 nM; FIG. 10). The potency seen in this biochemical assay was similar to that observed in the C. hominis cellular assay (Compound 11 ChPheRS [1% FBS] EC₅₀=10 nM, Tables 3 and 4), suggesting that inhibition of ChPheRS fully accounted for the observed cellular potency. The biochemical potency for Compound 11 with ChPheRS was also similar to that previously reported for cytosolic PfcPheRS (IC₅₀=23 nM), further suggesting a conserved MOA.

### Specific Embodiments

Non-limiting specific embodiments are described below each of which is considered to be within the present disclosure.

Specific Embodiment 1. A compound having the structure of formula (I):

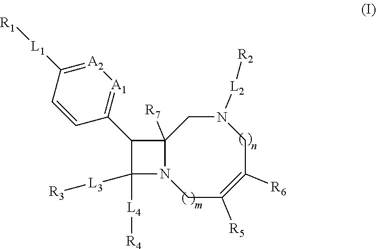

wherein the dashed bond ( [symbol] ) may be a single or double bond;
m is 0 (i.e., it is a bond) or 1;
n is 0, 1 or 2;
A₁ and A₂ are independently CH or N;
L₁ is absent (i.e., it is a bond), or —C≡C—;
L₂ is absent, alkylene (e.g., C₁-C₄ alkylene, methylene), —C(O)NR—; —SO₂—, or —C(O)—;
L₃ and L₄ are independently absent, alkylene (e.g., C₁-C₄ alkylene, methylene), or heteroalkylene (e.g., C₁-C₄ heteroalkylene);
R₁ is hydrogen, alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, C₁-C₈ heteroalkyl, C₁-C₅ heteroalkyl, C₃-C₁₂ heterocycloalkyl), halogen (e.g., fluoro, chloro), aryl (e.g., C₆-C₁₂ aryl, phenyl), heteroaryl (e.g., C₅-C₁₂ heteroaryl, pyridinyl), alkylaryl (e.g., C₇-C₁₄ alkylaryl, tolyl), arylalkyl (e.g., C₇-C₁₄ alkylaryl, benzyl), heteroalkylaryl (e.g., C₇-C₁₄ heteroalkylaryl), heteroarylalkyl (e.g., C₇-C₁₄ heteroarylalkyl), and R₁ has one or more (e.g., two, three, four, five) optional points of substitution;
R₂ is perfluoroalkyl, aryl (e.g., C₆-C₁₂ aryl, phenyl), arylalkyl (e.g., C₇-C₁₄ alkylaryl, benzyl), alkylaryl (e.g., C₇-C₁₄ alkylaryl, tolyl), alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, C₁-C₈ heteroalkyl, C₁-C₅ heteroalkyl, C₃-C₁₂ heterocycloalkyl), or heteroaryl (e.g., C₅-C₁₂ heteroaryl, pyridinyl), and R₂ has one or more (e.g., two, three, four, five) optional points of substitution (e.g., with alkoxy, fluoroalkoxy);
R₃ and R₄ are independently hydrogen, —OH, —OR, —S(O)₂R, —N(R)S(O)₂R, —C(O)R, —N(R)C(O) R, —N(R)₂, or heterocyclyl, and R₃ and/or R₄ has one or more (e.g., two, three, four, five) optional points of substitution;
R₅ and R₆ are independently selected from hydrogen and —OH;
R₇ is hydrogen, —CH₂OH, or —CH₂OR;
R is independently selected at each occurrence from hydrogen and alkyl (e.g., C₁-C₁₂ alkyl, C₁-C₈ alkyl, C₁-C₅ alkyl, C₃-C₁₂ cycloalkyl), wherein each R has one or more (e.g., two, three, four, five) optional points of substitution (e.g., with OH, with C(O)OH, —CN, —NH₂, —N(R_{A})₂); and
R_{A} is independently selected at each occurrence from hydrogen and lower alkyl (e.g., C₁-C₄ alkyl, methyl, ethyl, propyl, isopropyl);
wherein

a) -L₃-R₃ and -L₄-R₄ are each not hydrogen; and/or

b) R₇ is-CH₂OR₈; wherein R₈ is alkyl (e.g., C₁-C₁₂ alkyl, C₁-C₈ alkyl, C₁-C₅ alkyl, C₃-C₁₂ cycloalkyl) having one or more (e.g., two, three, four, five) optional points of substitution (e.g., with OH, C(O)OH, —CN, —NH₂, —N(R_{A})₂); or

pharmaceutically acceptable salts thereof; or
prodrugs of any of the foregoing.

Specific Embodiment 2. The compound according to Specific Embodiment 1 wherein the compound has the structure of formula (Ia):

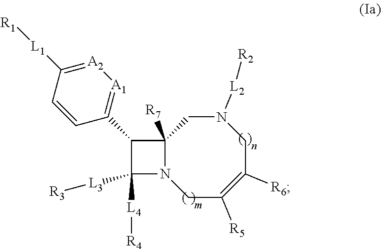

or
pharmaceutically acceptable salts thereof; or
prodrugs of any of the foregoing.

Specific Embodiment 3. The compound according to Specific Embodiment 1 or 2, wherein L₄ is alkylene.

Specific Embodiment 4. The compound according to Specific Embodiment 1 or 2, wherein L₄ is methylene.

Specific Embodiment 5. The compound according to any one of Specific Embodiments 1-4, wherein the compound has the structure of formula (II):

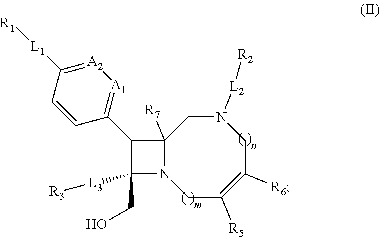

or
pharmaceutically acceptable salts thereof; or
prodrugs of any of the foregoing.

Specific Embodiment 6. The compound according to any one of Specific Embodiments 1-4, wherein the compound has the structure of formula (IIa):

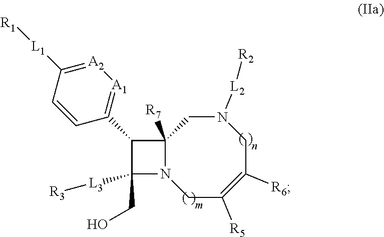

or
pharmaceutically acceptable salts thereof; or
prodrugs of any of the foregoing.

Specific Embodiment 7. The compound according to any one of Specific Embodiments 1-6, wherein R₇ is —CH₂—OR₈.

Specific Embodiment 8. The compound according to any one of Specific Embodiments 1-7, wherein the compound has the structure according to formula (III):

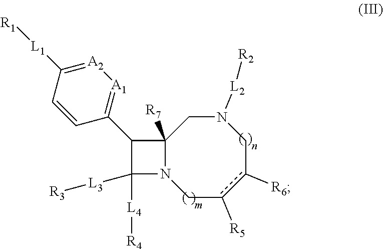

or
pharmaceutically acceptable salts thereof; or
prodrugs of any of the foregoing.

Specific Embodiment 9. The compound according to any one of Specific Embodiments 1-7, wherein the compound has the structure of formula (IIIa):

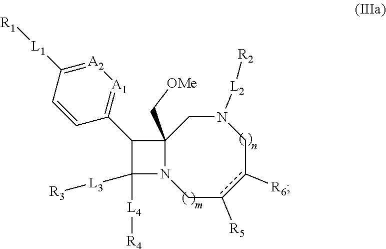

or
pharmaceutically acceptable salts thereof; or
prodrugs of any of the foregoing.

Specific Embodiment 10. The compound according to any one of Specific Embodiments 1-7, wherein the compound has the structure of formula (IIIb):

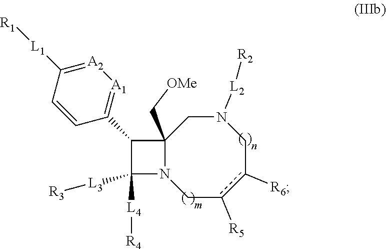

or
pharmaceutically acceptable salts thereof; or
prodrugs of any of the foregoing.

Specific Embodiment 11. The compound according to any one of Specific Embodiments 1-10, wherein R₃ is a group —O(CH₂)ₚC(O)OH or —NH(CH₂)ₚC(O)OH, wherein p is one, two, three, four, or five.

Specific Embodiment 12. A compound having the structure of:

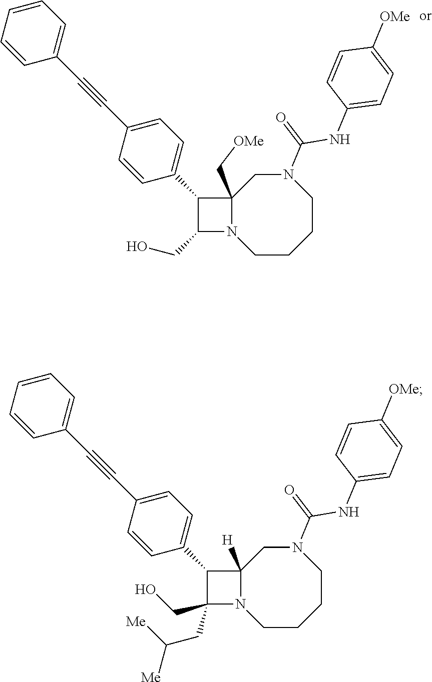 [SMILES: COC[C@@]12CN(C(=O)Nc3ccc(OC)cc3)CCCCN1[C@H](CO)[C@@H]2c1ccc(C#Cc2ccccc2)cc1 ; COc1ccc(NC(=O)N2CCCCN3[C@@H](C2)[C@@H](c2ccc(C#Cc4ccccc4)cc2)[C@@]3(CO)CC(C)C)cc1]

or
pharmaceutically acceptable salts thereof; or
prodrugs of any of the foregoing.

Specific Embodiment 13. A method of treatment or prophylaxis of a parasitic disease caused by a parasite from the genus Cryptosporidium in a subject in need thereof comprising administration to the subject the compound according to any one of Specific Embodiments 1-12, or a pharmaceutical salt thereof; or a prodrug of any of the foregoing.

Specific Embodiment 14. A method of treatment or prophylaxis of a parasitic disease caused by a parasite from the genus Cryptosporidium in a subject in need thereof comprising administration to the subject a compound having the structure of formula (IV):

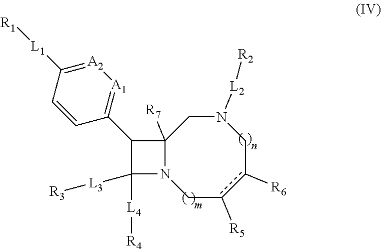

wherein the dashed bond ( [symbol] ) may be a single or double bond;
m is 0 (i.e., it is a bond) or 1;
n is 0, 1 or 2;
A₁ and A₂ are independently CH or N;
L₁ is absent (i.e., it is a bond), or —C≡C—;
L₂ is absent, alkylene (e.g., C₁-C₄ alkylene, methylene), heteroalkylene (e.g., C₁-C₄ heteroalkylene), —C(O)NR—; —SO₂—, or —C(O)—;
L₃ and L₄ are independently absent, alkylene (e.g., C₁-C₄ alkylene, methylene), or heteroalkylene (e.g., C₁-C₄ heteroalkylene);
R₁ is hydrogen, alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, C₁-C₈ heteroalkyl, C₁-C₅ heteroalkyl, C₃-C₁₂ heterocycloalkyl), halogen (e.g., fluoro, chloro), aryl (e.g., C₆-C₁₂ aryl, phenyl), or heteroaryl (e.g., C₅-C₁₂ heteroaryl, pyridinyl), and R₁ has one or more (e.g., two, three, four, five) optional points of substitution;
R₂ is perfluoroalkyl, aryl (e.g., C₆-C₁₂ aryl, phenyl), arylalkyl (e.g., C₇-C₁₄ alkylaryl, benzyl), alkyl (e.g., C₁-C₁₂ alkyl, C₁-C₈ alkyl, C₁-C₅ alkyl, C₃-C₁₂ cycloalkyl), or heteroaryl (e.g., C₅-C₁₂ heteroaryl, pyridinyl), and R₂ has one or more (e.g., two, three, four, five) optional points of substitution (e.g., with alkoxy, fluoroalkoxy);
R₃ and R₄ are independently hydrogen, —OH, —OR, —S(O)₂R, —N(R)S(O)₂R, —C(O)R, —N(R)C(O) R, —N(R)₂, or heterocyclyl, and R₃ and/or R₄ has one or more (e.g., two, three, four, five) optional points of substitution;
R₅ and R₆ are independently selected from hydrogen and —OH; wherein R₅ and R₆ are not each —OH;
R₇ is hydrogen, —CH₂OH, or —CH₂OR;
R is independently selected at each occurrence from hydrogen and alkyl (e.g., C₁-C₁₂ alkyl, C₁-C₈ alkyl, C₁-C₅ alkyl, C₃-C₁₂ cycloalkyl), wherein each R has one or more (e.g., two, three, four, five) optional points of substitution (e.g., with OH, with C(O)OH, —CN, —NH₂, —N(R_{A})₂); and
R_{A} is independently selected at each occurrence from hydrogen and lower alkyl (e.g., C₁-C₄ alkyl, methyl, ethyl, propyl, isopropyl); or
pharmaceutically acceptable salts thereof; or
prodrugs of any of the foregoing.

Specific Embodiment 15. The method according to Specific Embodiment 14, wherein the compound does not have the structure:

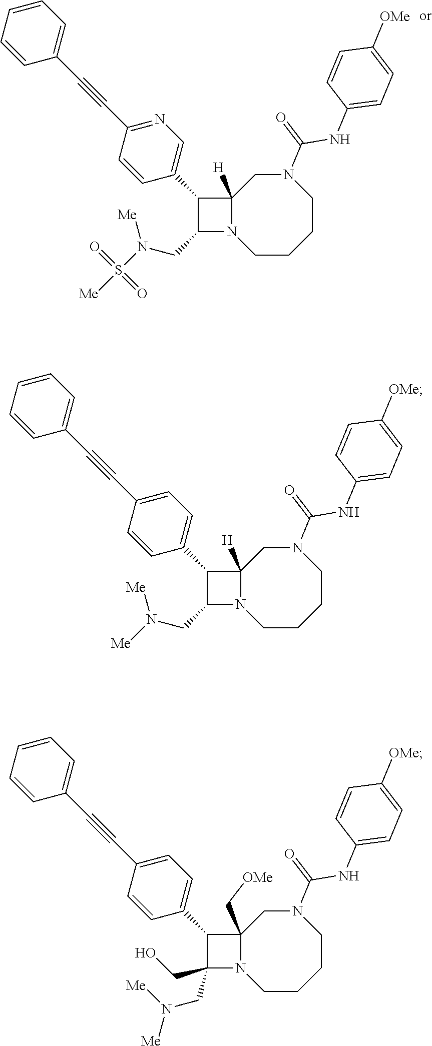 [SMILES: COc1ccc(NC(=O)N2CCCCN3[C@H](CN(C)S(C)(=O)=O)[C@H](c4ccc(C#Cc5ccccc5)nc4)[C@@H]3C2)cc1 ; COc1ccc(NC(=O)N2CCCCN3[C@H](CN(C)C)[C@H](c4ccc(C#Cc5ccccc5)cc4)[C@@H]3C2)cc1 ; COC[C@@]12CN(C(=O)Nc3ccc(OC)cc3)CCCCN1[C@](CO)(CN(C)C)[C@@H]2c1ccc(C#Cc2ccccc2)cc1]

or
pharmaceutically acceptable salts thereof; or
prodrugs of any of the foregoing.

Specific Embodiment 16. The method according to Specific Embodiment 14, wherein R₅ and R₆ are each hydrogen.

Specific Embodiment 17. The method according to any one of Specific Embodiments 13-16, wherein L₂ is —C(O)NH—.

Specific Embodiment 18. The method according to any one of Specific Embodiments 12-17, wherein R₂ is aryl having one or more (e.g., two, three, four, five) optional points of substitution (e.g., with alkoxy, fluoroalkoxy).

Specific Embodiment 19. The method according to any one of Specific Embodiments 12-18, wherein R₂ is para substituted phenyl.

Specific Embodiment 20. The method according to any one of Specific Embodiments 12-18, wherein R₂ is 4-methoxyphenyl.

Specific Embodiment 21. The method according to any one of Specific Embodiments 12-20, wherein R₁ is aryl or heteroaryl.

Specific Embodiment 22. The method according to any one of Specific Embodiments 12-20, wherein R₁ is phenyl.

Specific Embodiment 23. The method according to any one of Specific Embodiments 14-22, wherein the compound has the structure of:

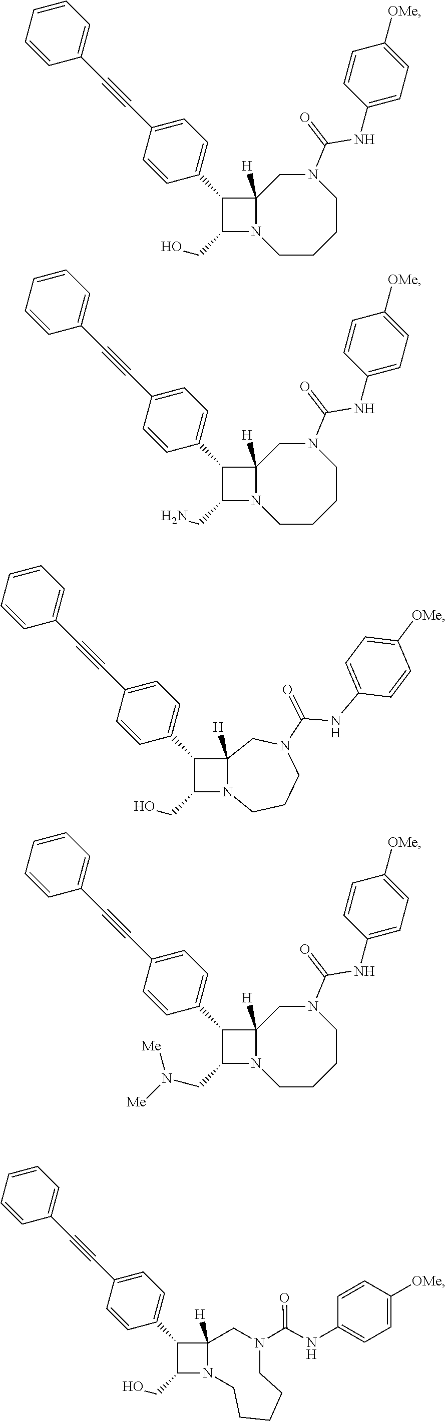 [SMILES: COc1ccc(NC(=O)N2CCCCN3[C@H](CO)[C@H](c4ccc(C#Cc5ccccc5)cc4)[C@@H]3C2)cc1 ; COc1ccc(NC(=O)N2CCCCN3[C@H](CN)[C@H](c4ccc(C#Cc5ccccc5)cc4)[C@@H]3C2)cc1 ; COc1ccc(NC(=O)N2CCCN3[C@H](CO)[C@H](c4ccc(C#Cc5ccccc5)cc4)[C@@H]3C2)cc1 ; COc1ccc(NC(=O)N2CCCCN3[C@H](CN(C)C)[C@H](c4ccc(C#Cc5ccccc5)cc4)[C@@H]3C2)cc1 ; COc1ccc(NC(=O)N2CCCCCN3[C@H](CO)[C@H](c4ccc(C#Cc5ccccc5)cc4)[C@@H]3C2)cc1]

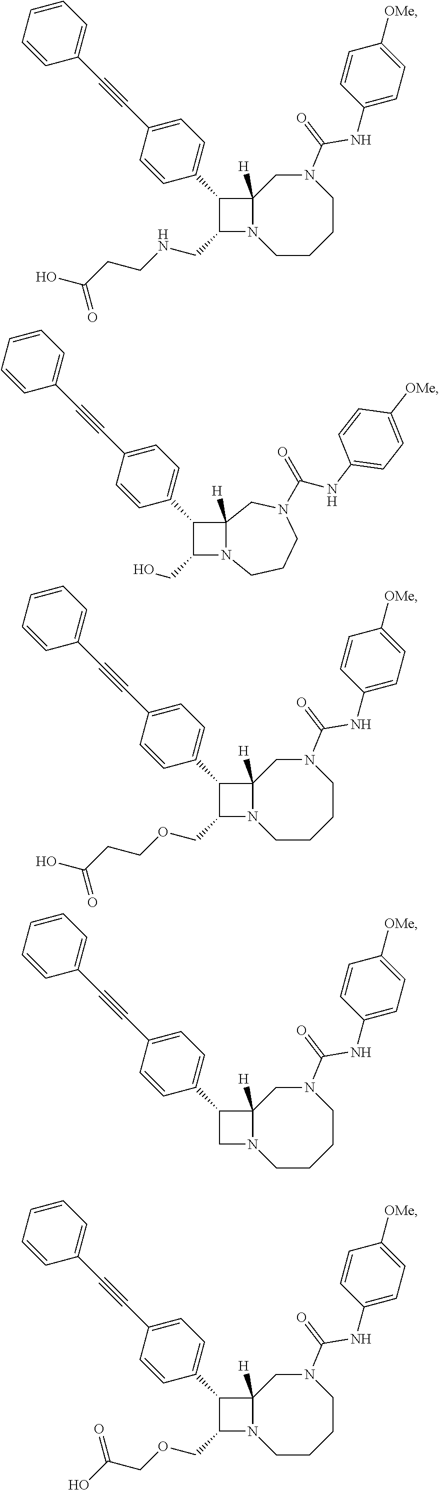

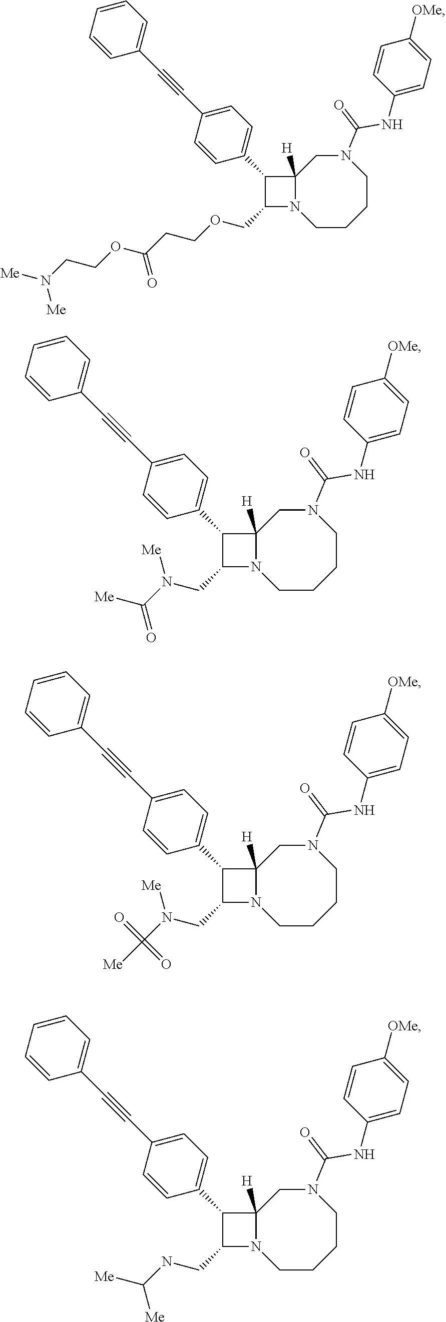

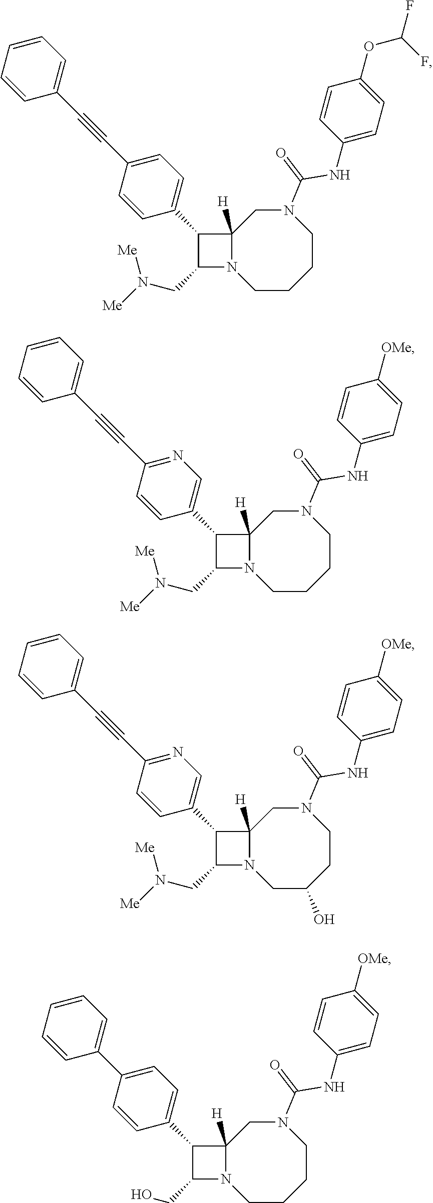 [SMILES: CN(C)C[C@@H]1[C@H](c2ccc(C#Cc3ccccc3)cc2)[C@@H]2CN(C(=O)Nc3ccc(OC(F)F)cc3)CCCCN12 ; COc1ccc(NC(=O)N2CCCCN3[C@H](CN(C)C)[C@H](c4ccc(C#Cc5ccccc5)nc4)[C@@H]3C2)cc1 ; COc1ccc(NC(=O)N2CC[C@H](O)CN3[C@H](CN(C)C)[C@H](c4ccc(C#Cc5ccccc5)nc4)[C@@H]3C2)cc1 ; COc1ccc(NC(=O)N2CCCCN3[C@H](CO)[C@H](c4ccc(-c5ccccc5)cc4)[C@@H]3C2)cc1]

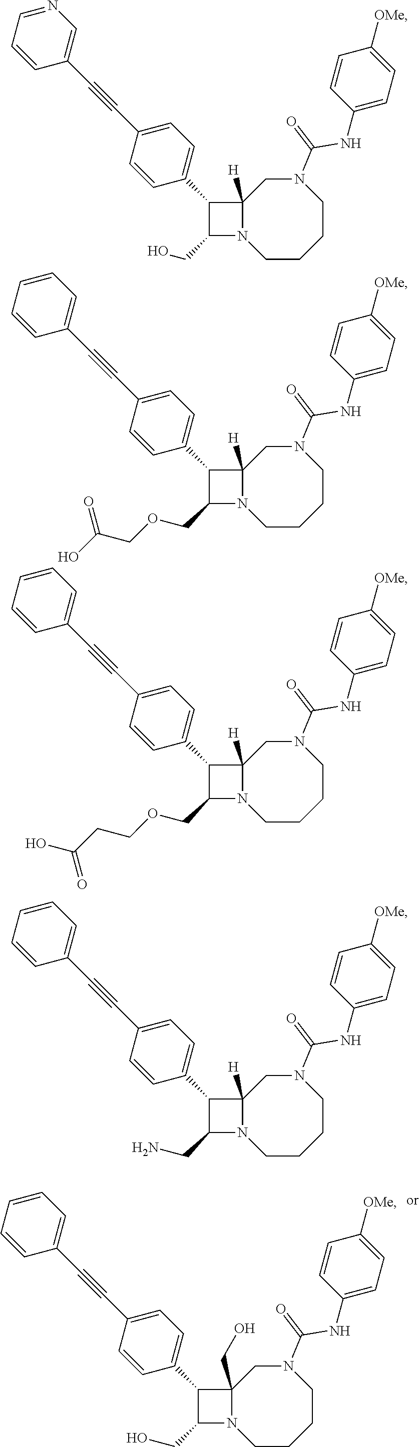

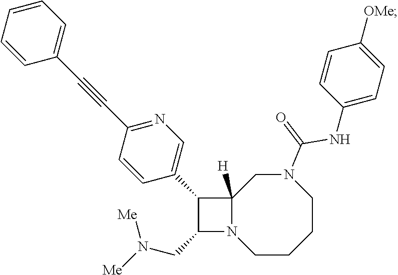 [SMILES: COc1ccc(NC(=O)N2CCCCN3[C@H](CN(C)C)[C@H](c4ccc(C#Cc5ccccc5)nc4)[C@@H]3C2)cc1]

or
pharmaceutically acceptable salts thereof; or
prodrugs of any of the foregoing.

Specific Embodiment 24. The method according to any one of Specific Embodiments 13 or 16-22, wherein the compound has the structure of formula (I).

Specific Embodiment 25. The method according to any one of Specific Embodiments 13 or 16-22, wherein the compound has the structure:

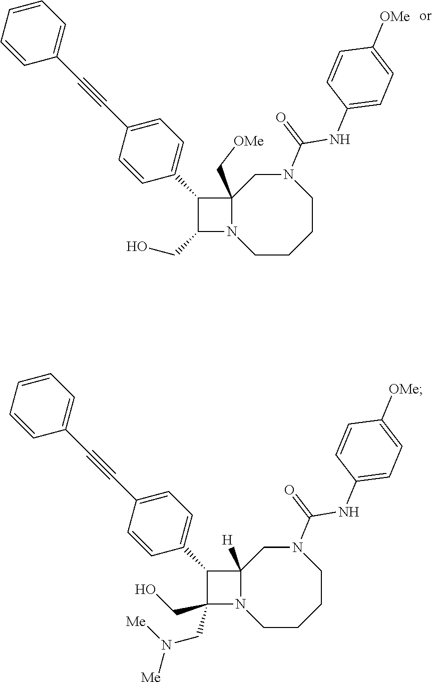 [SMILES: COC[C@@]12CN(C(=O)Nc3ccc(OC)cc3)CCCCN1[C@H](CO)[C@@H]2c1ccc(C#Cc2ccccc2)cc1 ; COc1ccc(NC(=O)N2CCCCN3[C@@H](C2)[C@@H](c2ccc(C#Cc4ccccc4)cc2)[C@@]3(CO)CN(C)C)cc1]

or
pharmaceutically acceptable salts thereof; or
prodrugs of any of the foregoing.

Specific Embodiment 26. The method according to any one of Specific Embodiments 14-22, wherein the compound has the structure:

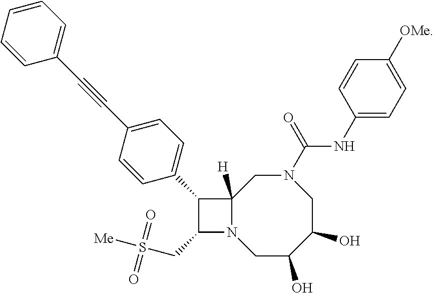 [SMILES: COc1ccc(NC(=O)N2C[C@@H](O)[C@@H](O)CN3[C@H](CS(C)(=O)=O)[C@H](c4ccc(C#Cc5ccccc5)cc4)[C@@H]3C2)cc1]

Specific Embodiment 27. The method of any one of Specific Embodiments 12-26, wherein the parasitic disease is cryptosporidiosis.

Specific Embodiment 28. The method of Specific Embodiment 27, wherein the cryptosporidiosis is carried by C. parvum or C. hominis.

Specific Embodiment 29. The method according to Specific Embodiment 28, wherein the C. parvum or C. hominis comprises wild type PheRS gene.

Specific Embodiment 30. The method of any one of Specific Embodiments 12-29, wherein the subject is human.

Specific Embodiment 31. The method of any one of Specific Embodiments 12-29, wherein the subject is not human.

Specific Embodiment 32. The method of Specific Embodiment 31, wherein the subject is a mouse, rat, rabbit, non-human primate, lizards, geckos, cow, calf, sheep, lamb, horse, foal, pig, or piglet.

Specific Embodiment 33. The method of any one of Specific Embodiments 12-32, wherein the subject is administered a pharmaceutical composition comprising a therapeutically effective amount of the compound.

Specific Embodiment 34. A pharmaceutical composition comprising a pharmaceutically acceptable excipient and the compound according to any one of Specific Embodiments 1-12, or a pharmaceutical salt thereof; or a prodrug of any of the foregoing.

Specific Embodiment 35. A pharmaceutical composition comprising a pharmaceutically acceptable excipient and a compound a compound having the structure of formula (IV):

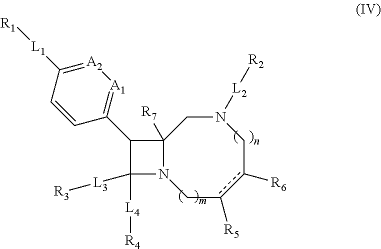

wherein the dashed bond ( [symbol] ) may be a single or double bond;
m is 0 (i.e., it is a bond) or 1;
n is 0, 1 or 2;
A₁ and A₂ are independently CH or N;
L₁ is absent (i.e., it is a bond), or —C≡C—;
L₂ is absent, alkylene, —C(O)NR—; —SO₂—, or —C(O)—;
L₃ and L₄ are independently absent, alkylene, or heteroalkylene;
R₁ is hydrogen, alkyl, heteroalkyl, halogen, aryl, heteroaryl, or cycloalkyl, and R₁ has one or more (e.g., two, three, four, five) optional points of substitution;
R₂ is perfluoroalkyl, aryl, arylalkyl, alkyl, heteroaryl, or cycloalkyl, and R₂ has one or more (e.g., two, three, four, five) optional points of substitution (e.g., with alkoxy, fluoroalkoxy);
R₃ and R₄ are independently hydrogen, —OH, —OR, —S(O)₂R, —N(R)S(O)₂R, —C(O)R, —N(R)C(O) R, —N(R)₂, or heterocyclyl, and R₃ and/or R₄ has one or more (e.g., two, three, four, five) optional points of substitution;
R₅ and R₆ are independently selected from hydrogen and —OH; wherein R₅ and R₆ are not each —OH;
R₇ is hydrogen, —CH₂OH, or —CH₂OR;
R is independently selected at each occurrence from hydrogen and alkyl, wherein R has one or more (e.g., two, three, four, five) optional points of substitution (e.g., with OH, with C(O)OH, —CN, —NH₂, —N(R_{A})₂); and
R_{A} is independently selected at each occurrence from hydrogen and lower alkyl; or
pharmaceutically acceptable salts thereof; or
prodrugs of any of the foregoing.

Specific Embodiment 36. The pharmaceutical composition according to Specific Embodiment 34 or 35, wherein the pharmaceutical composition is formulated as a veterinary composition.

Specific Embodiment 37. The pharmaceutical composition according to any one of Specific Embodiments 34-36, wherein the compound is present in a therapeutically effective amount to treat a disease caused by a parasite from the genus Cryptosporidium.

Specific Embodiment 38. The pharmaceutical composition according to any one of Specific Embodiments 34-37, wherein the disease is cryptosporidiosis.

Specific Embodiment 39. A method of inhibiting or preventing the growth of a population of parasites from the genus Cryptosporidium in a medium comprising contacting the population with a compound having the structure of formula (IV):

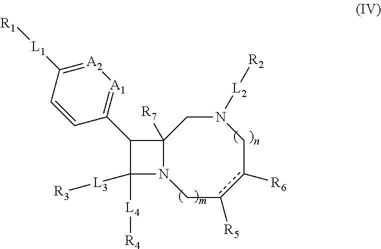

wherein the dashed bond ( [symbol] ) may be a single or double bond;
m is 0 (i.e., it is a bond) or 1;
n is 0, 1 or 2;
A₁ and A₂ are independently CH or N;
L₁ is absent (i.e., it is a bond), or —C≡C—;
L₂ is absent, alkylene, —C(O)NR—; —SO₂—, or —C(O)—;
L₃ and L₄ are independently absent, alkylene, or heteroalkylene;
R₁ is hydrogen, alkyl, heteroalkyl, halogen, aryl, heteroaryl, or cycloalkyl, and R₁ has one or more (e.g., two, three, four, five) optional points of substitution;
R₂ is perfluoroalkyl, aryl, arylalkyl, alkyl, heteroaryl, or cycloalkyl, and R₂ has one or more (e.g., two, three, four, five) optional points of substitution (e.g., with alkoxy, fluoroalkoxy);
R₃ and R₄ are independently hydrogen, —OH, —OR, —S(O)₂R, —N(R)S(O)₂R, —C(O)R, —N(R)C(O) R, —N(R)₂, or heterocyclyl, and R₃ and/or R₄ has one or more (e.g., two, three, four, five) optional points of substitution;
R₅ and R₆ are independently selected from hydrogen and —OH; wherein R₅ and R₆ are not each —OH;
R₇ is hydrogen, —CH₂OH, or —CH₂OR;
R is independently selected at each occurrence from hydrogen and alkyl, wherein R has one or more (e.g., two, three, four, five) optional points of substitution (e.g., with OH, with C(O)OH, —CN, —NH₂, —N(R_{A})₂); and
R_{A} is independently selected at each occurrence from hydrogen and lower alkyl; or
pharmaceutically acceptable salts thereof; or
prodrugs of any of the foregoing.

Specific Embodiment 40. The method according to Specific Embodiment 39, wherein the medium is in vitro.

Specific Embodiment 41. The method according to Specific Embodiment 39, wherein the medium is in vivo.

Specific Embodiment 42. The method according to any one of Specific Embodiments 39-41, wherein the compound has the structure of formula (I):

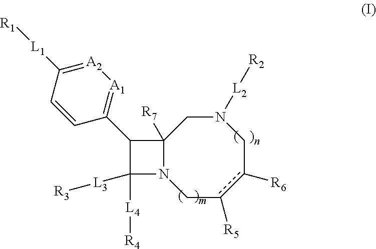

wherein the dashed bond ( [symbol] ) may be a single or double bond;
m is 0 (i.e., it is a bond) or 1;
n is 0, 1 or 2;
A₁ and A₂ are independently CH or N;
L₁ is absent (i.e., it is a bond), or —C≡C—;
L₂ is absent, alkylene (e.g., C₁-C₄ alkylene, methylene), —C(O)NR—; —SO₂—, or —C(O)—;
L₃ and L₄ are independently absent, alkylene (e.g., C₁-C₄ alkylene, methylene), or heteroalkylene (e.g., C₁-C₄ heteroalkylene);
R₁ is hydrogen, alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, C₁-C₈ heteroalkyl, C₁-C₅ heteroalkyl, C₃-C₁₂ heterocycloalkyl), halogen (e.g., fluoro, chloro), aryl (e.g., C₆-C₁₂ aryl, phenyl), heteroaryl (e.g., C₅-C₁₂ heteroaryl, pyridinyl), alkylaryl (e.g., C₇-C₁₄ alkylaryl, tolyl), arylalkyl (e.g., C₇-C₁₄ alkylaryl, benzyl), heteroalkylaryl (e.g., C₇-C₁₄ heteroalkylaryl), heteroarylalkyl (e.g., C₇-C₁₄ heteroarylalkyl), and R₁ has one or more (e.g., two, three, four, five) optional points of substitution;
R₂ is perfluoroalkyl, aryl (e.g., C₆-C₁₂ aryl, phenyl), arylalkyl (e.g., C₇-C₁₄ alkylaryl, benzyl), alkylaryl (e.g., C₇-C₁₄ alkylaryl, tolyl), alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, C₁-C₅ heteroalkyl, C₁-C₅ heteroalkyl, C₃-C₁₂ heterocycloalkyl), or heteroaryl (e.g., C₅-C₁₂ heteroaryl, pyridinyl), and R₂ has one or more (e.g., two, three, four, five) optional points of substitution (e.g., with alkoxy, fluoroalkoxy);
R₃ and R₄ are independently hydrogen, —OH, —OR, —S(O)₂R, —N(R)S(O)₂R, —C(O)R, —N(R)C(O) R, —N(R)₂, or heterocyclyl, and R₃ and/or R₄ has one or more (e.g., two, three, four, five) optional points of substitution;
R₅ and R₆ are independently selected from hydrogen and —OH;
R₇ is hydrogen, —CH₂OH, or —CH₂OR;
R is independently selected at each occurrence from hydrogen and alkyl (e.g., C₁-C₁₂ alkyl, C₁-C₅ alkyl, C₁-C₅ alkyl, C₃-C₁₂ cycloalkyl), wherein each R has one or more (e.g., two, three, four, five) optional points of substitution (e.g., with OH, with C(O)OH, —CN, —NH₂, —N(R_{A})₂); and
R_{A} is independently selected at each occurrence from hydrogen and lower alkyl (e.g., C₁-C₄ alkyl, methyl, ethyl, propyl, isopropyl);
wherein:

a) -L₃-R₃ and -L₄-R₄ are each not hydrogen; and/or

b) R₇ is-CH₂OR₈; wherein R₈ is alkyl (e.g., C₁-C₁₂ alkyl, C₁-C₈ alkyl, C₁-C₅ alkyl, C₃-C₁₂ cycloalkyl) having one or more (e.g., two, three, four, five) optional points of substitution (e.g., with OH, with C(O)OH, —CN, —NH₂, —N(R_{A})₂); or

pharmaceutically acceptable salts thereof; or
prodrugs of any of the foregoing.

Specific Embodiment 43. The method according to any one of Specific Embodiments 39-42, wherein the Cryptosporidium parasites comprise wild type PheRS.

Specific Embodiment 44. The method according to any one of Specific Embodiments 39-43, wherein the Cryptosporidium parasites are C. parvum or C. hominis.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims. In various embodiments, the compound is not a compound disclosed in Table 1 of Int'l. Pub. No. WO 2015/070204 or Table 1 of Int'l. Pub. No. WO 2018/175385, each hereby incorporated by reference in their entirety.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

What is claimed is:

1. A compound having the structure of formula (I): 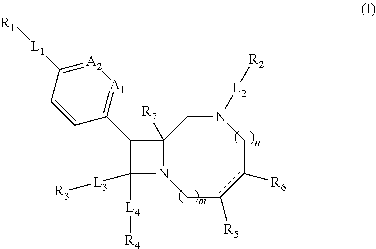
wherein the dashed bond ( [symbol] ) may be a single or double bond;
m is 0 (i.e., it is a bond) or 1;
n is 0, 1 or 2;
A₁ and A₂ are independently CH or N;
L₁ is absent (i.e., it is a bond), or —C≡C—;
L₂ is absent, alkylene, —C(O)NR—; —SO₂—, or —C(O)—;
L₃ and L₄ are independently absent, alkylene, or heteroalkylene;
R₁ is hydrogen, alkyl, heteroalkyl, halogen, aryl, heteroaryl, alkylaryl, arylalkyl, heteroalkylaryl, heteroarylalkyl, and R₁ has one or more optional points of substitution;
R₂ is perfluoroalkyl, aryl, arylalkyl, alkylaryl, alkyl, heteroalkyl, or heteroaryl, and R₂ has one or more optional points of substitution;
R₃ and R₄ are independently hydrogen, —OH, —OR, —S(O)₂R, —N(R)S(O)₂R, —C(O)R, —N(R)C(O) R, —N(R)₂, or heterocyclyl, and R₃ and/or R₄ has one or more optional points of substitution;
R₅ and R₆ are independently selected from hydrogen and —OH;
R₇ is hydrogen, —CH₂OH, or —CH₂OR; and
R is independently selected at each occurrence from hydrogen and alkyl, wherein each R has one or more optional points of substitution;
wherein
a) -L₃-R₃ and -L₄-R₄ are each not hydrogen; and/or
b) R₇ is-CH₂OR₈; wherein R₈ is alkyl having one or more optional points of substitution; or
pharmaceutically acceptable salts thereof; or
prodrugs of any of the foregoing.

2. The compound according to claim 1, wherein said compound has the structure of formula (II):

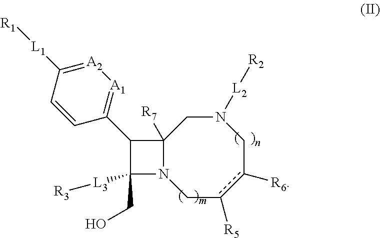

3. The compound according to claim 1, wherein said compound has the structure of formula (IIa):

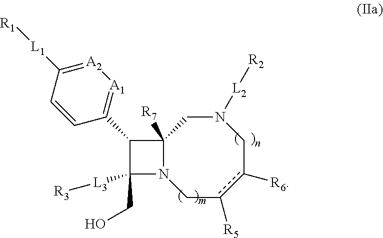

4. The compound according to claim 1, wherein R₇ is —CH₂—OR₈.

5. The compound according to claim 1, wherein said compound has the structure of formula (IIIa):

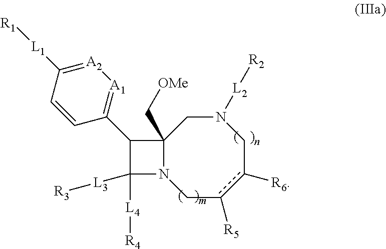

6. The compound according to claim 1, wherein R₃ is a group —O(CH₂)ₚC(O)OH or —NH(CH₂)ₚC(O)OH, wherein p is one, two, three, four, or five.

7. A compound having the structure of: 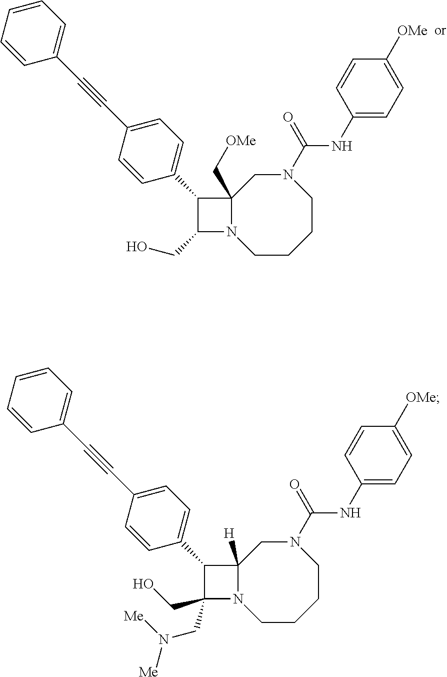 [SMILES: COC[C@@]12CN(C(=O)Nc3ccc(OC)cc3)CCCCN1[C@H](CO)[C@@H]2c1ccc(C#Cc2ccccc2)cc1 ; COc1ccc(NC(=O)N2CCCCN3[C@@H](C2)[C@@H](c2ccc(C#Cc4ccccc4)cc2)[C@@]3(CO)CN(C)C)cc1]
pharmaceutically acceptable salts thereof; or
prodrugs of any of the foregoing.

8. A method of treatment or prophylaxis of a parasitic disease caused by a parasite from the genus Cryptosporidium in a subject in need thereof comprising administration to said subject the compound according to claim 1, or a pharmaceutical salt thereof; or a prodrug of any of the foregoing.

9. A method of treatment or prophylaxis of a parasitic disease caused by a parasite from the genus Cryptosporidium in a subject in need thereof comprising administration to said subject a compound having the structure of formula (IV): 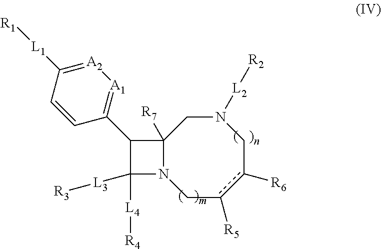
wherein the dashed bond ( [symbol] ) may be a single or double bond;
m is 0 (i.e., it is a bond) or 1;
n is 0, 1 or 2;
A₁ and A₂ are independently CH or N;
L₁ is absent (i.e., it is a bond), or —C≡C—;
L₂ is absent, alkylene, heteroalkylene, —C(O)NR—; —SO₂—, or —C(O)—;
L₃ and L₄ are independently absent, alkylene, or heteroalkylene;
R₁ is hydrogen, alkyl, heteroalkyl, halogen, aryl, or heteroaryl, and R₁ has one or more optional points of substitution;
R₂ is perfluoroalkyl, aryl, arylalkyl, alkyl, or heteroaryl, and R₂ has one or more (e.g., two, three, four, five) optional points of substitution (e.g., with alkoxy, fluoroalkoxy);
R₃ and R₄ are independently hydrogen, —OH, —OR, —S(O)₂R, —N(R)S(O)₂R, —C(O)R, —N(R)C(O)R, —N(R)₂, or heterocyclyl, and R₃ and/or R₄ has one or more (e.g., two, three, four, five) optional points of substitution;
R₅ and R₆ are independently selected from hydrogen and —OH; wherein R₅ and R₆ are not each —OH;
R₇ is hydrogen, —CH₂OH, or —CH₂OR; and
R is independently selected at each occurrence from hydrogen and alkyl, wherein each R has one or more optional points of substitution; or
prodrugs of any of the foregoing.

10. The method according to claim 9, wherein said compound has the structure of: 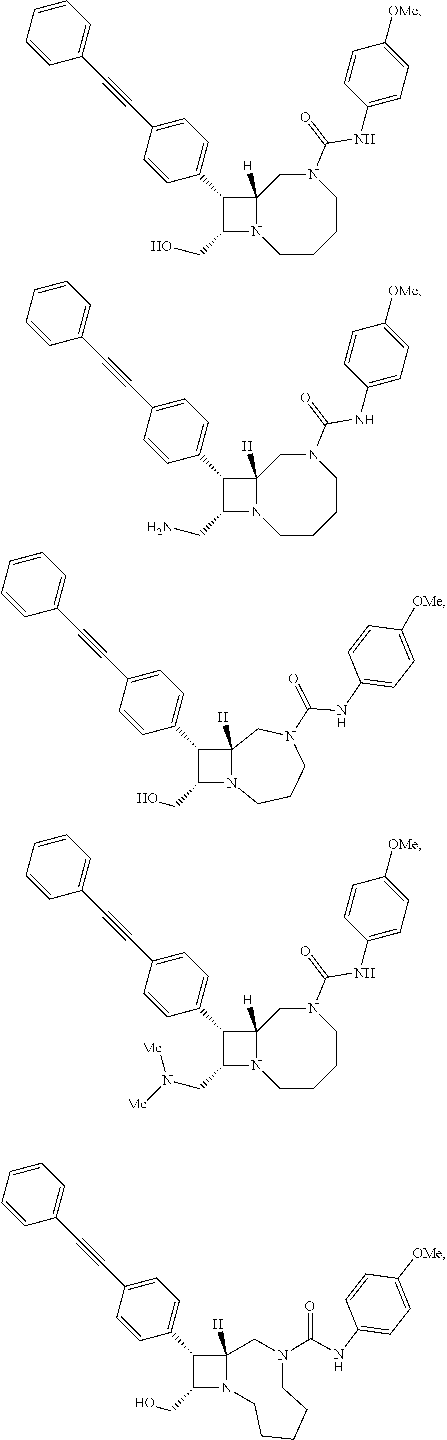 [SMILES: COc1ccc(NC(=O)N2CCCCN3[C@H](CO)[C@H](c4ccc(C#Cc5ccccc5)cc4)[C@@H]3C2)cc1 ; COc1ccc(NC(=O)N2CCCCN3[C@H](CN)[C@H](c4ccc(C#Cc5ccccc5)cc4)[C@@H]3C2)cc1 ; COc1ccc(NC(=O)N2CCCN3[C@H](CO)[C@H](c4ccc(C#Cc5ccccc5)cc4)[C@@H]3C2)cc1 ; COc1ccc(NC(=O)N2CCCCN3[C@H](CN(C)C)[C@H](c4ccc(C#Cc5ccccc5)cc4)[C@@H]3C2)cc1 ; COc1ccc(NC(=O)N2CCCCCN3[C@H](CO)[C@H](c4ccc(C#Cc5ccccc5)cc4)[C@@H]3C2)cc1] 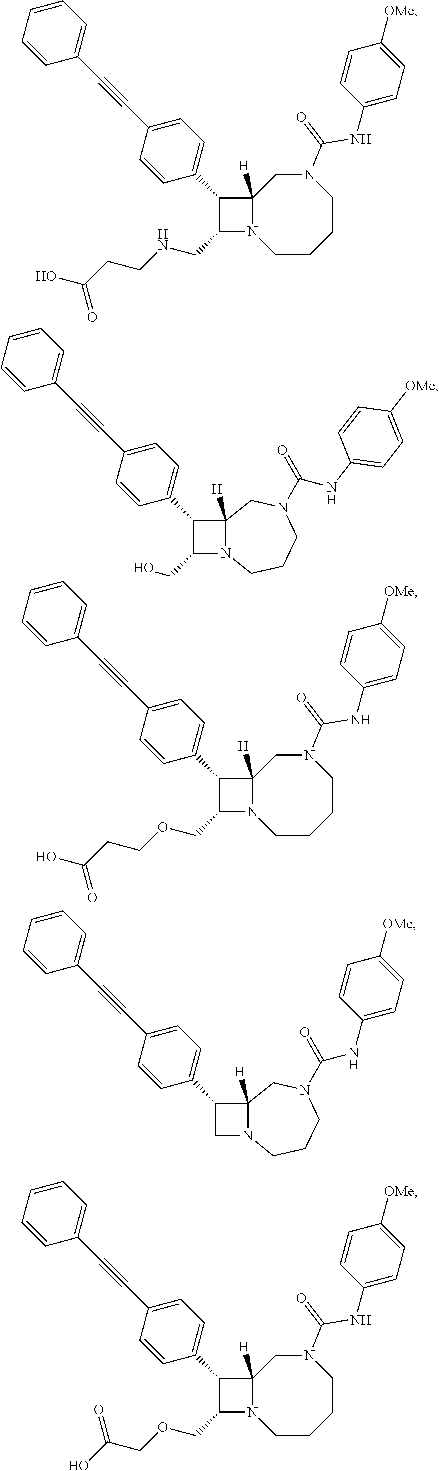 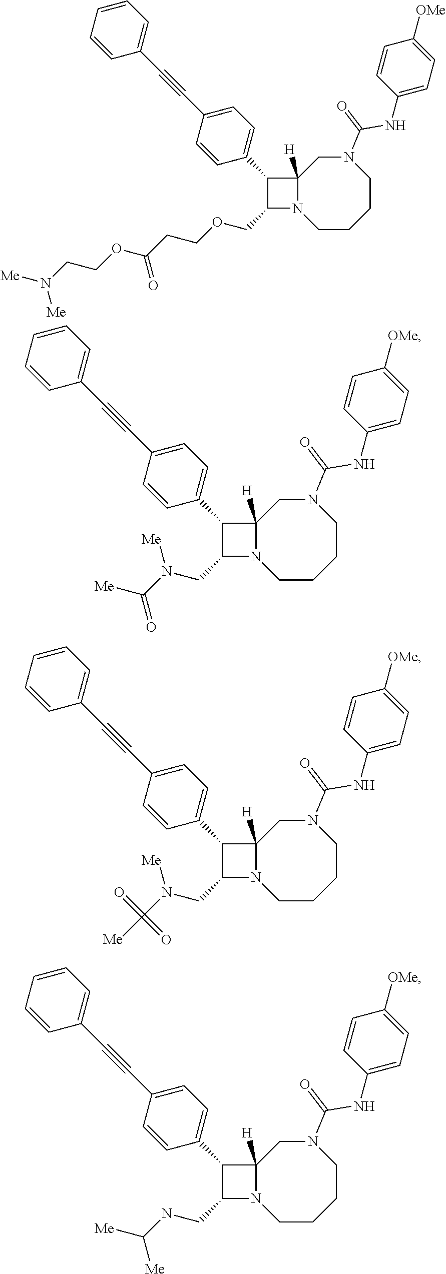 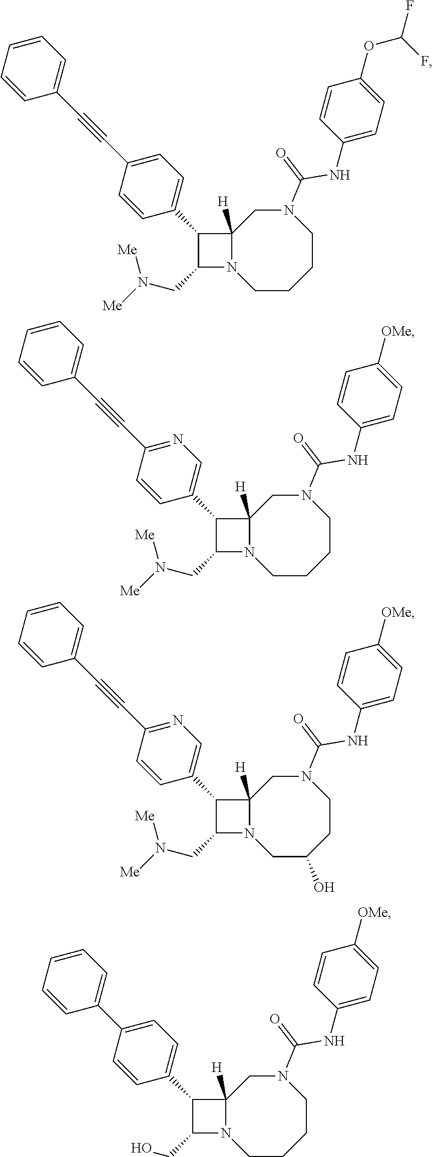 [SMILES: CN(C)C[C@@H]1[C@H](c2ccc(C#Cc3ccccc3)cc2)[C@@H]2CN(C(=O)Nc3ccc(OC(F)F)cc3)CCCCN12 ; COc1ccc(NC(=O)N2CCCCN3[C@H](CN(C)C)[C@H](c4ccc(C#Cc5ccccc5)nc4)[C@@H]3C2)cc1 ; COc1ccc(NC(=O)N2CC[C@H](O)CN3[C@H](CN(C)C)[C@H](c4ccc(C#Cc5ccccc5)nc4)[C@@H]3C2)cc1 ; COc1ccc(NC(=O)N2CCCCN3[C@H](CO)[C@H](c4ccc(-c5ccccc5)cc4)[C@@H]3C2)cc1] 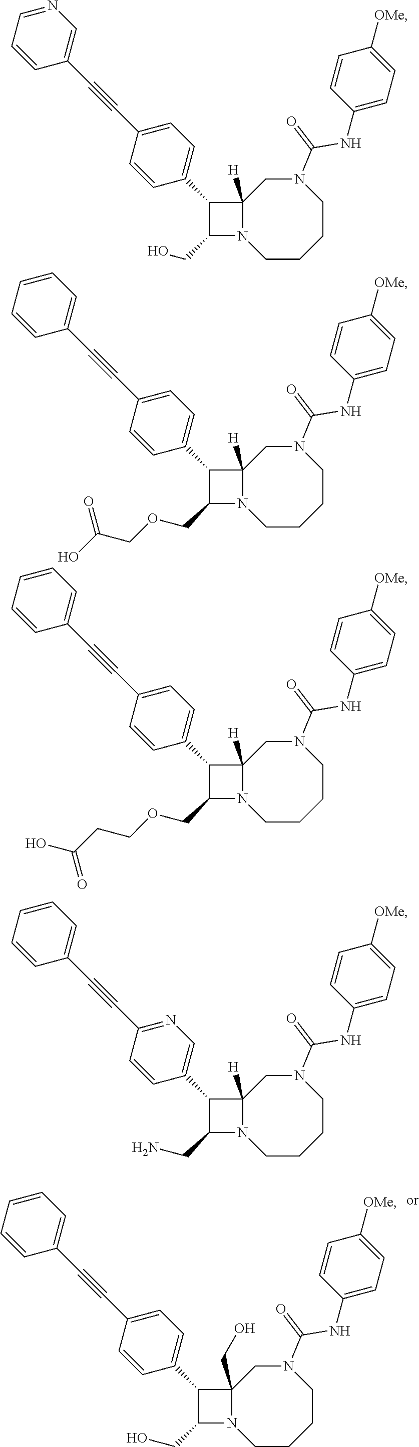 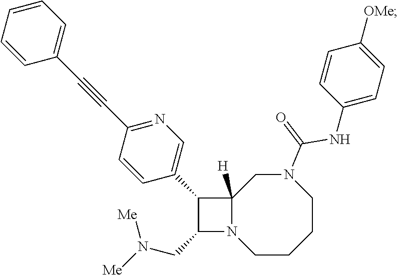 [SMILES: COc1ccc(NC(=O)N2CCCCN3[C@H](CN(C)C)[C@H](c4ccc(C#Cc5ccccc5)nc4)[C@@H]3C2)cc1]
or
pharmaceutically acceptable salts thereof; or
prodrugs of any of the foregoing.

11. The method according to claim 9, wherein said compound has the structure of formula (I).

12. The method according to claim 8, wherein said compound has the structure: 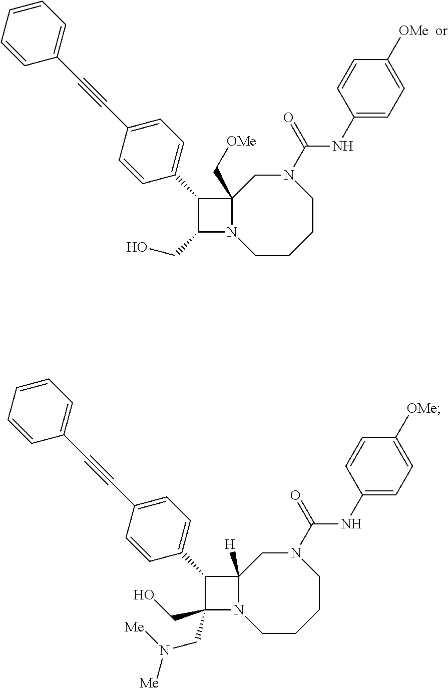 [SMILES: COC[C@@]12CN(C(=O)Nc3ccc(OC)cc3)CCCCN1[C@H](CO)[C@@H]2c1ccc(C#Cc2ccccc2)cc1 ; COc1ccc(NC(=O)N2CCCCN3[C@@H](C2)[C@@H](c2ccc(C#Cc4ccccc4)cc2)[C@@]3(CO)CN(C)C)cc1]
or
pharmaceutically acceptable salts thereof; or
prodrugs of any of the foregoing.

13. The method according to claim 9, wherein said compound has the structure: 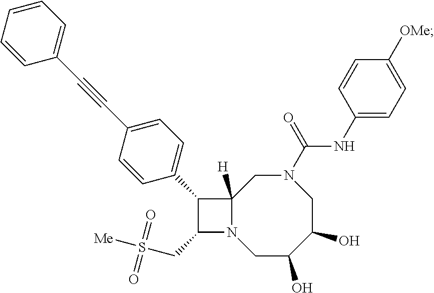 [SMILES: COc1ccc(NC(=O)N2C[C@@H](O)[C@@H](O)CN3[C@H](CS(C)(=O)=O)[C@H](c4ccc(C#Cc5ccccc5)cc4)[C@@H]3C2)cc1]
or
pharmaceutically acceptable salts thereof; or
prodrugs of any of the foregoing.

14-20. (canceled)

21. A pharmaceutical composition comprising a pharmaceutically acceptable excipient and the compound according to claim 1, or a pharmaceutical salt thereof; or a prodrug of any of the foregoing.

22. A pharmaceutical composition comprising a pharmaceutically acceptable excipient and a compound a compound having the structure of formula (IV): 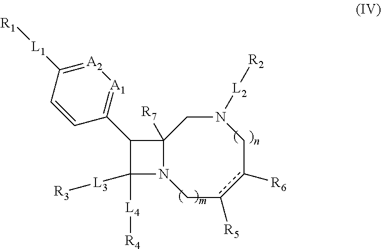
wherein the dashed bond ( [symbol] ) may be a single or double bond;
m is 0 (i.e., it is a bond) or 1;
n is 0, 1 or 2;
A₁ and A₂ are independently CH or N;
L₁ is absent (i.e., it is a bond), or —C≡C—;
L₂ is absent, alkylene, —C(O)NR—; —SO₂—, or —C(O)—;
L₃ and L₄ are independently absent, alkylene, or heteroalkylene;
R₁ is hydrogen, alkyl, heteroalkyl, halogen, aryl, heteroaryl, or cycloalkyl, and R₁ has one or more optional points of substitution;
R₂ is perfluoroalkyl, aryl, arylalkyl, alkyl, heteroaryl, or cycloalkyl, and R₂ has one or more (e.g., two, three, four, five) optional points of substitution (e.g., with alkoxy, fluoroalkoxy);
R₃ and R₄ are independently hydrogen, —OH, —OR, —S(O)₂R, —N(R)S(O)₂R, —C(O)R, —N(R)C(O) R, —N(R)₂, or heterocyclyl, and R₃ and/or R₄ has one or more (e.g., two, three, four, five) optional points of substitution;
R₅ and R₆ are independently selected from hydrogen and —OH; wherein R₅ and R₆ are not each —OH;
R₇ is hydrogen, —CH₂OH, or —CH₂OR;
R is independently selected at each occurrence from hydrogen and alkyl, wherein R has one or more optional points of substitution; or
pharmaceutically acceptable salts thereof; or
prodrugs of any of the foregoing.

23. (canceled)

24. The pharmaceutical composition according to claim 21, wherein said compound is present in a therapeutically effective amount to treat a disease caused by a parasite from the genus Cryptosporidium.

25. (canceled)

26. A method of inhibiting or preventing the growth of a population of parasites from the genus Cryptosporidium in a medium comprising contacting said population with a compound having the structure of formula (IV): 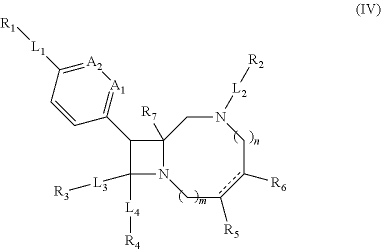
wherein the dashed bond ( [symbol] ) may be a single or double bond;
m is 0 (i.e., it is a bond) or 1;
n is 0, 1 or 2;
A₁ and A₂ are independently CH or N;
L₁ is absent (i.e., it is a bond), or —C≡C—;
L₂ is absent, alkylene, —C(O)NR—; —SO₂—, or —C(O)—;
L₃ and L₄ are independently absent, alkylene, or heteroalkylene;
R₁ is hydrogen, alkyl, heteroalkyl, halogen, aryl, heteroaryl, or cycloalkyl, and R₁ has one or more optional points of substitution;
R₂ is perfluoroalkyl, aryl, arylalkyl, alkyl, heteroaryl, or cycloalkyl, and R₂ has one or more optional points of substitution;
R₃ and R₄ are independently hydrogen, —OH, —OR, —S(O)₂R, —N(R)S(O)₂R, —C(O)R, —N(R)C(O) R, —N(R)₂, or heterocyclyl, and R₃ and/or R₄ has one or more (e.g., two, three, four, five) optional points of substitution;
R₅ and R₆ are independently selected from hydrogen and —OH; wherein R₅ and R₆ are not each —OH;
R₇ is hydrogen, —CH₂OH, or —CH₂OR;
R is independently selected at each occurrence from hydrogen and alkyl, wherein R has one or more optional points of substitution or
pharmaceutically acceptable salts thereof; or
prodrugs of any of the foregoing.

27. The method according to claim 26, wherein said compound has the structure of formula (I): 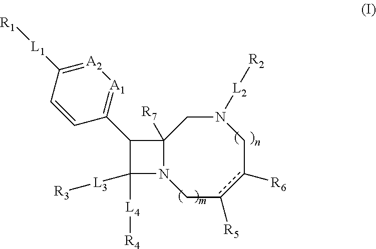
wherein the dashed bond ( [symbol] ) may be a single or double bond;
m is 0 (i.e., it is a bond) or 1;
n is 0, 1 or 2;
A₁ and A₂ are independently CH or N;
L₁ is absent (i.e., it is a bond), or —C≡C—;
L₂ is absent, alkylene, —C(O)NR—; —SO₂—, or —C(O)—;
L₃ and L₄ are independently absent, alkylene, or heteroalkylene;
R₁ is hydrogen, alkyl, heteroalkyl, halogen, aryl, heteroaryl, alkylaryl, arylalkyl, heteroalkylaryl, heteroarylalkyl, and R₁ has one or more optional points of substitution;
R₂ is perfluoroalkyl, aryl, arylalkyl, alkylaryl, alkyl, heteroalkyl, or heteroaryl, and R₂ has one or more optional points of substitution;
R₃ and R₄ are independently hydrogen, —OH, —OR, —S(O)₂R, —N(R)S(O)₂R, —C(O)R, —N(R)C(O) R, —N(R)₂, or heterocyclyl, and R₃ and/or R₄ has one or more optional points of substitution;
R₅ and R₆ are independently selected from hydrogen and —OH;
R₇ is hydrogen, —CH₂OH, or —CH₂OR; and
R is independently selected at each occurrence from hydrogen and alkyl, wherein each R has one or more optional points of substitution;
wherein:
a) -L₃-R₃ and -L₄-R₄ are each not hydrogen; and/or
b) R₇ is-CH₂OR₈; wherein R₈ is alkyl having one or more optional points of substitution; or
pharmaceutically acceptable salts thereof; or
prodrugs of any of the foregoing.

28. The method according to claim 26, wherein said Cryptosporidium parasites comprise wild type PheRS.

29. The method according to claim 26, wherein said Cryptosporidium parasites are C. parvum or C. hominis.


SEQUENCE LISTING (27 sequences)

SEQ ID NO: 1 - DNA, 21 bp, Artificial Sequence
  Description of Artificial Sequence Synthetic primer
tagagattggaggttgttcct

SEQ ID NO: 2 - DNA, 21 bp, Artificial Sequence
  Description of Artificial Sequence Synthetic primer
ctccaccaactaagaacggcc

SEQ ID NO: 3 - DNA, 24 bp, Artificial Sequence
  Description of Artificial Sequence Synthetic primer
gttgggcaatcacaacgatgtcag

SEQ ID NO: 4 - DNA, 24 bp, Artificial Sequence
  Description of Artificial Sequence Synthetic primer
aaacctgacatcgttgtgattgcc

SEQ ID NO: 5 - DNA, 120 bp, Artificial Sequence
  Description of Artificial Sequence Synthetic primer
ctaggctgacatcgttgtgattgcatgggggctttctcttgaaaggccaacgatgattagttataatatcccaaatatacgcgatctatttagctttaaagcaaaaatattttcataagg

SEQ ID NO: 6 - DNA, 120 bp, Artificial Sequence
  Description of Artificial Sequence Synthetic primer
cgcgccttatgaaaatatttttgctttaaagctaaatagatcgcgtatatttgggatattataactaatcatcgttggcctttcaagagaaagcccccatgcaatcacaacgatgtcagc

SEQ ID NO: 7 - DNA, 120 bp, Artificial Sequence
  Description of Artificial Sequence Synthetic primer
ctaggctgacatcgttgtgattgcatggggggtttctcttgaaaggccaacgatgattagttataatatcccaaatatacgcgatctatttagctttaaagcaaaaatattttcataagg

SEQ ID NO: 8 - DNA, 120 bp, Artificial Sequence
  Description of Artificial Sequence Synthetic primer
cgcgccttatgaaaatatttttgctttaaagctaaatagatcgcgtatatttgggatattataactaatcatcgttggcctttcaagagaaaccccccatgcaatcacaacgatgtcagc

SEQ ID NO: 9 - DNA, 74 bp, Artificial Sequence
  Description of Artificial Sequence Synthetic primer
gttggaaattccggcttatttagaccagaaatgcttagaccactcgggtttccatctgacatcgttgtgattgc

SEQ ID NO: 10 - DNA, 71 bp, Artificial Sequence
  Description of Artificial Sequence Synthetic primer
agtgattataatctaaaaaacaaacattaattcgaaaagtctacattaggaattaagataaaaagaaaaac

SEQ ID NO: 11 - DNA, 36 bp, Artificial Sequence
  Description of Artificial Sequence Synthetic primer
cgacaagaatcttacacttgcagatctaatcggaac

SEQ ID NO: 12 - DNA, 23 bp, Artificial Sequence
  Description of Artificial Sequence Synthetic primer
ctttggatcggagttacggacac

SEQ ID NO: 13 - DNA, 40 bp, Artificial Sequence
  Description of Artificial Sequence Synthetic primer
cagacttaaggcccgtatgcccgacggtgaagatcttgtc

SEQ ID NO: 14 - DNA, 39 bp, Artificial Sequence
  Description of Artificial Sequence Synthetic primer
cgaacccgagccatcagttcggaaaactgagatgctagc

SEQ ID NO: 15 - protein, 55 aa, Plasmodium falciparum
LEVGNSGIFRPEMLRSMGFSEEVSVIAWGLSLERPTMIKYNIKNIRDLFGYKSVV

SEQ ID NO: 16 - protein, 57 aa, Cryptosporidium parvum
IEVGNSGLFRPEMLRPLGFPSDIVVIAWGLSLERPTMISYNIPNIRDLFSFKAKIFS

SEQ ID NO: 17 - protein, 57 aa, Cryptosporidium hominis; identical to SEQ ID NO: 16

SEQ ID NO: 18 - DNA, 54 bp, Cryptosporidium parvum
gggtttccctctgacatcgttgtgattgcatgggggctttctcttgaaaggcca

SEQ ID NO: 19 - DNA, 54 bp, Cryptosporidium parvum
gggtttccctctgacatcgttgtgattgcatgggggctttctcttgaaagacca

SEQ ID NO: 20 - DNA, 54 bp, Artificial Sequence
  Description of Artificial Sequence Synthetic oligonucleotide
gggtttccctctgacatcgttgtgattgcatggggggtttctcttgaaagacca

SEQ ID NO: 21 - DNA, 123 bp, Cryptosporidium parvum
gggtttccctctgacatcgttgtgattgcatgggggctttctcttgaaaggccaacgatgattagttataatatcccaaatatacgcgatctatttagctttaaagcaaaaatattttcataa

SEQ ID NO: 22 - DNA, 123 bp, Cryptosporidium parvum
gggtttccatctgacatcgttgtgattgcatgggggctttctcttgaaaggccaacgatgattagttataatatcccaaatatacgcgatctatttagctttaaagcaaaaatattttcataa

SEQ ID NO: 23 - DNA, 123 bp, Artificial Sequence
  Description of Artificial Sequence Synthetic polynucleotide
gggtttccatctgacatcgttgtgattgcatggggggtttctcttgaaaggccaacgatgattagttataatatcccaaatatacgcgatctatttagctttaaagcaaaaatattttcataa

SEQ ID NO: 24 - protein, 5 aa, Cryptosporidium parvum
WGLSL

SEQ ID NO: 25 - DNA, 15 bp, Cryptosporidium parvum
tgggggctttctctt

SEQ ID NO: 26 - protein, 5 aa, Artificial Sequence
  Description of Artificial Sequence Synthetic peptide
WGVSL

SEQ ID NO: 27 - DNA, 15 bp, Artificial Sequence
  Description of Artificial Sequence Synthetic oligonucleotide
tggggggtttctctt